SEQUENCE LISTING (19 sequences)

SEQ ID NO: 1 - DNA, 2396 bp, Influenza B virus
  source 1..2396: mol_type = other DNA
agcagaagcggagcgttttcaagatgacattggctaaaattgaattgttaaaacaactgttaagggacaacgaagccaaaacagtattgaaacaaacaacggtagaccaatataacataataagaaaattcaatacatcaagaattgaaaagaacccttcattaaggatgaagtgggccatgtgttctaattttcccttggctctgaccaagggtgatatggcaaatagaatccccttggaatacaagggaatacaacttaaaacaaatgctgaagacataggaaccaaaggccaaatgtgctcaatagcagcagttacctggtggaatacatatggaccaataggagacactgaaggtttcgaaaaggtctacgaaagcttctttctcagaaagatgagacttgacaatgccacttggggccgaataacttttggcccagttgaaagagtaagaaaaagggtactgctaaaccctctcaccaaggaaatgcctccagatgaagcgagcaatgtgataatggaaatattgttccctaaggaagcaggaataccaagagaatctacttggatacatagggaactgataaaagaaaaaagagaaaaattgaaaggaacgatgataactcccattgtactggcatacatgcttgagagagaattggttgcccgaagaaggttcctgccagtggcaggagcaacatcagctgagttcatagaaatgctacactgcttacaaggtgaaaattggagacaaatatatcacccaggagggaataaactaactgaatctaggtctcaatcaatgattgtagcttgtagaaaaataatcagaagatcaatagtcgcatcaaacccactagagctagctgtagaaattgcaaacaagactgtgatagatactgaacctttaaaatcatgtctggcagccatagacggaggtgatgtagcctgtgacataatgagagctgcattaggactaaagatcagacaaagacaaagatttggacggcttgaactaaagagaatatcaggaagaggattcaaaaatgatgaagaaatattgatcgggaacggaacaatacagaagattggaatatgggacggagaagaggagttccatgtaagatgtggtgaatgcaggggaatattaaaaaagagcaaaatgagaatggaaaaactactaataaattcagccaaaaaggaggacatgaaagatttaataatcttgtgcatggtattttctcaagacactaggatgttccaaggagtgagaggagaaataaattttcttaatcgagcaggccaacttttatctccaatgtaccaactccaaagatattttttgaataggagcaacgatctttttgatcaatgggggtatgaggaatcacccaaagcaagtgaactacatgggataaatgaattaatgaatgcatctgattatacgttgaaaggggttgtagtaacaaaaaatgtgattgatgactttagttctactgaaacagaaaaagtatctataacaaaaaatcttagtttaataaaaagaactggggaagtcataatgggggctaatgacgtaagtgaattagaatcacaagctcagctaatgataacatatgatacacctaagatgtgggagatgggaacaaccaaagaactggtgcaaaacacctaccaatgggtgctaaaaaatttggtaacactgaaggctcagtttcttctaggaaaagaagacatgttccaatgggatgcatttgaagcatttgaaagcataatcccccagaagatggctggccagtacagtggatttgcaagggcagtgctcaaacaaatgagagaccaagaggttatgaaaactgaccagttcataaagttgttgcctttctgtttctcaccaccaaaattaaggagcaatggggagccttatcaattcttgaggcttatattgaagggaggaggagaaaatttcatcgaagtaaggaaagggtcccctctattctcctacaatccacaaacagaagtcctaactatatgcggcagaatgatgtcattaaaagggaaaattgaagatgaagaaaggaatagatcaatggggaatgcagtattggcaggctttctcgttagtggcaagtatgacccagatcttggagatttcaaaactattgaagaacttgaaaagctaaaaccgggggagaaagcaaacatcttactttatcaaggaaagcccgttaaagtagttaaaaggaaaagatatagtgctttatccaatgacatttcacaaggaattaagagacaaagaatgacagttgagtccatggggtgggccttgagctaatataaatttatccattaattcaataaacacaattgagtgaaaaatgctcgtgtttctact

SEQ ID NO: 2 - DNA, 2369 bp, Influenza B virus
  source 1..2369: mol_type = other DNA
agcagaagcggagcctttaagatgaatataaatccttattttctcttcatagatgtacccatacaggcagcaatttcaacaacattcccatacaccggtgttcccccttattcccatggaacgggaacaggctacacaatagacaccgtgatcagaacacatgagtactcaaacaagggaaaacagtacatttctgatgttacaggatgtacaatggtagatccaacaaatgggccattacccgaagacaatgagccgagtgcctatgcacaattagattgcgttctggaggctttggatagaatggatgaagaacatccaggtctgtttcaagcagcctcacagaatgccatggaggcactaatggtcacaactgtagacaaattaacccaggggagacagacttttgattggacagtatgcagaaaccaacctgctgcaacggcactgaacacaacaataacctcttttaggttgaatgatttgaatggagccgacaagggtggattagtacccttttgccaagatatcattgattcattggacagacctgaaatgactttcttctcagtaaagaatataaagaaaaaattgcctgctaaaaacagaaagggtttcctcataaagagaataccaatgaaggtaaaagacagaataaccagagtggaatacatcaaaagagcattatcattaaacacaatgacaaaagatgctgaaagaggcaaactaaaaagaagagcgattgccaccgctggaatacaaatcagagggtttgtattagtagttgaaaacttggctaaaaatatctgtgaaaatctagaacaaagtggtttgccagtaggtggaaacgagaagaaggccaaactgtcaaatgcagtggccaaaatgctcagtaactgcccaccaggagggatcagcatgacagtaacaggagacaataccaaatggaatgaatgcttaaatccaagaatctttttggctatgactgaaagaataaccagagacagcccaatttggttccgggatttttgtagtatagcaccggtcttgttctccaataaaatagccagattgggaaaagggtttatgataacaagcaaaacaaaaagactgaaggctcaaataccttgtcctgatctgtttagtataccattagaaagatataatgaagaaacaagggcaaaattgaaaaagctgaaaccattcttcaatgaagaaggaacggcatctttgtcgcctgggatgatgatgggaatgtttaatatgctatctaccgtgttgggagtagccgcactaggtatcaaaaacattggaaacaaagaatacttatgggatggactgcaatcttctgatgattttgctctgtttgttaatgcaaaagatgaagagacatgtatggaaggaataaacgacttttaccgaacatgtaaactattgggaataaacatgagcaaaaagaaaagttactgtaatgaaactggaatgtttgaatttacaagcatgttctacagagatggatttgtatctaattttgcaatggaacttccttcatttggagttgctggagtaaatgaatcagcagatatggcaataggaatgacaataataaagaacaatatgatcaacaatgggatgggtccagcaacagcacaaacagccatacaattattcatagctgattatagatacacctacaaatgccacaggggagattccaaagtggaaggaaagagaatgaaaattataaaggagctatgggaaaacactaaaggaagagatggtctgttagtagcagatggtgggcctaacatttacaatttgagaaacttgcatatcccagaaatagtattaaagtacaacctaatggaccctgaatacaaagggcggttacttcatcctcaaaatccctttgtaggacatttgtctattgagggcatcaaagaggcagatataaccccagcacatggtccagtaaagaaaatggactatgatgcggtgtctggaactcatagttggagaaccaaaaggaacagatctatactaaacactgatcagaggaacatgattcttgaggaacaatgctacgctaagtgttgcaacctttttgaggcctgttttaacagtgcatcatacaggaaaccagtaggtcagcacagcatgcttgaggctatggcccacagattaagaatggatgcacgactagattatgaatcaggaagaatgtcaaaggatgattttgagaaagcaatggctcaccttggtgagattgggtacatataagcttcgaagatgtctatggggttattggtcatcattgaatacatgcggtacacaaatgattaaaatgaaaaaaggctcgtgtttctact

SEQ ID NO: 3 - DNA, 2308 bp, Influenza B virus
  source 1..2308: mol_type = other DNA
agcagaagcggtgcgtttgatttgccataatggatacttttattacaagaaacttccagactacaataatacaaaaggccaaaaacacaatggcagaatttagtgaagatcctgaattacaaccagcaatgctattcaacatctgcgtccatctggaggtctgctatgtaataagtgatatgaattttcttgatgaagaaggaaaagcatatacagcattagaaggacaaggaaaagaacaaaacttgagaccacaatatgaagtgattgagggaatgccaagaaacatagcatggatggttcaaagatccttagcccaagagcatgggatagagactccaaggtatctggctgatttgttcgattataaaactaagaggtttatagaagttggaataacaaagggattggctgatgattacttctggaaaaagaaagaaaagctggggaatagcatggaactgatgatattcagctacaatcaagactattcgttaagtaatgaatcctcattggatgaggaaggaaaagggagagtgctaagcagactcacagaacttcaggctgagttaagtctgaaaaatctatggcaagttctcataggagaagaagatattgaaaaaggaattgacttcaaacttggacaaacaatatctaaactaagggatatatctgttccagctggtttctccaattttgaaggaatgaggagctacatagacaatatagatcctaaaggagcaatagagagaaatctagcaaggatgtctcccttagtatcagttacacctaaaaagttgaaatgggaggacctaagaccaatagggcctcacatttacaaccatgagctaccagaagttccatataatgcctttcttctaatgtctgatgaattggggctggctaatatgactgaagggaagtccaagaaaccgaagaccttagccaaagagtgcctagaaaagtactcaacactacgggatcaaactgacccaatattaataatgaaaagcgaaaaagctaatgaacacttcctatggaaactgtggagggactgtgtaaatacaataagtaatgaggaaacaagtaacgaattacagaaaaccaattatgccaagtgggccacaggagatggattaacatatcagaaaataatgaaagaagtagcaatagatgacgaaacaatgtaccaagaagagcccaaaatacctaacaaatgtagagtggctgcttgggttcaaacagagatgaatctattgagcactctgacaagtaaaagggccctggatctgccagaaatagggccagacgtagcacccgtggagcatgtagggagtgaaagaaggaaatactttgttaatgaaatcaactactgtaaggcctctaccgttatgatgaagtatgtactttttcacacttcattattaaatgaaagcaatgccagcatgggaaaatataaagtaataccaataaccaacagagtagtaaatgaaaagggagaaagttttgacatgctttatggtctagcggttaaagggcaatctcatctgaggggagatactgatgttgtaacagttgtgactttcgaatttagtagtacagatcccagagtggactcaggaaagtggccaaaatatactgtatttagaattggctccttatttgtgagtgggagggaaaaatctgtatacctatattgccgagtgaatggtacaaataagatccaaatgaaatggggaatggaagctagaagatgtctgcttcaatcaatgcaacaaatggaagcaattgttgaacaagaatcatcgatacaaggatatgacatgaccaaagcttgtttcaagggagacagagtgaatagtccaaaaactttcagtattgggactcaagaaggaaaactagtgaaaggatcctttgggaaagcactaagagtaatattcaccaaatgtttgatgcactatgtatttggaaatgcccaattggaggggtttagtgccgaatctaggagacttctactgttaattcaggcattaaaagacagaaagggcccttgggtattcgacttagagggaatgtattctggaatagaagaatgtattagtaacaacccttgggtaatacagagtgcatactggtttaatgaatggttgggctttgaaaaagaggggagtaaagtattagaatcagtagatgaaataatggatgaatgaaagaagggcatagtgctcaatttggtactattttgttcattatgtatctaaacatccaataaaaagaattgagaattaaaaatgcacgtgtttctact

SEQ ID NO: 4 - DNA, 1840 bp, Influenza B virus
  source 1..1840: mol_type = other DNA
agcagaagcacagcattttcttgtgaacttcaagtaccaacaaaaaactgaaaatcaaaatgtccaacatggacattgacggcatcaacactggaataattgacaaaacaccagaagaaataacttccggaaccagtggggcaaccagaccaatcatcagaccagcaacccttgccccaccaagcaacaaacgaaccagaaacccatccccggaaagggcaaccacaagcagtgaagctgatgtcggaaggaaaacccaaaagaaacaaactccgacagagataaagaagagcgtctacaatatggtagtgaaactgggtgaattctacaaccagatgatggtcaaagctggactcaacgatgacatggagagaaacctaatccaaaatgcacatgctgtggaaagaattctattggctgctactgatgacaagaaaactgaattccaaaagaaaaagaatgccagagacgtcaaagaagggaaagaagaaatagaccacaacaaaacaggaggcaccttttacaagatggtaagagatgataaaaccatctacttcagccctataagaattacctttttaaaagaagaggtgaaaacaatgtacaaaaccaccatggggagtgacggtttcagtggactaaatcacatcatgattgggcattcacagatgaacgatgtctgtttccaaagatcaaaggcactaaaaagagttggacttgacccttcattaatcagtacttttgcaggaagcacactccccagaagatcaggtacaactggtgttgcgaccaaaggaggtggaactttagtggcagaagccattcgatttataggaagagcaatggcagacagagggctattgagagacatcagagccaagacggcctatgaaaagattcttctgaatctgaaaaacaagtgctctgcgccccaacaaaaggctctggttgatcaagtgatcggaagtagaaatccagggattgcagacatagaagatctcaccctgcttgctcgaagtatggtcgttgttaggccctctgtagcaagcaaagtggtgcttcccataagcatctatgctaaaatacctcaactggggttcaacgttgaagaatactctatggttgggtatgaagccatggctctttataatatggcaacacctgtttccatattaagaatgggagacgatgcaaaagataaatcacaattattcttcatgtcttgctttggagctgcctatgaagacctaagagttctgtctgcactaacaggcacggaattcaagcctaggtcagcattaaagtgcaaaggtttccacgttccagcaaaggagcaagtggaaggaatgggggcagctctgatgtccatcaagctccagttttgggctccaatgaccagatctggggggaatgaagtaggtggagacggagggtctggtcaaataagttgcagccccgtgtttgcagtagaaagacctattgctctaagcaagcaagctgtaagaagaatgctgtcaatgaatattgagggacgtgatgcagatgtcaaaggaaatctactcaagatgatgaatgattcaatggctaagaaaaccaatggaaatgctttcattgggaagaaaatgttccaaatatcagacaaaaacaaaaccaatcccgttgagattccaattaagcagaccatccccagtttcttctttgggagggacacagcagaggattatgatgacctcgattattaaagcaacaaaatagacactatggctgtgattgtttcagtacgtttggaatgtgggtgtttactcttattgaaataaatgtaaaaaatgctgttgtttct

SEQ ID NO: 5 - DNA, 1190 bp, Influenza B virus
  source 1..1190: mol_type = other DNA
agcagaagcacgcactttcttaaaatgtcgctgtttggagacacaattgcctacctgctttcactaacagaagatggagaaggcaaagcagaactagcggaaaaattacactgttggttcggtgggaaagaattcgatctagactctgctttggaatggataaaaaacaaaagatgcctaactgatatacaaaaagcactaattggtgcctctatctgctttttgaaacccaaagaccaagaaagaaaaagaagattcatcacagagcccctgtcaggaatgggaacaacagcaacaaaaaagaaaggcctgattctagctgaaagaaaaatgagaagatgtgtgagttttcatgaggcatttgaaatagcagaaggccatgaaagctcagcactactatattgtctcatggtcatgtacctgaaccctggaaattattcaatgcaagtaaaactaggaacgctctgtgctttatgcgagaaacaagcatcacattcacacagagctcatagcagagcagcaagatcttcagtgcctggagtgaggcgagaaatgcaaatggtttcagctatgaacacagcaaaaacaatgaatggaatggggaagggagaagacgtccaaaaactggcagaagagctgcaaagcaacattggagtattgagatctctgggggcaagtcaaaagaatggagaaggaattgcaaaggatgtaatggaagtgctaaagcagagctccatgggaaattcagctcttgtgaagaaatacctataatgctcgagccatttcagattctttcaatttgctctttcattttatcggctctccatttcatgggctggacaatagggcatttaaatcaaataaaaagaggagtaaacctaaaaatacgaataagaaatccaaataaagagacaataaatagagaggtatcaattttgagacacagttaccaaaaagaaatccaagccaaagaaacaataaaggaagtactctctgacaacatggagagattgagtgaccacatagtaattgaggggctttctgctgaagagataataaaaatgggtgaaacagttttggaggtagaagaattgcattaaacccaattttcaccgtatttcttactatgcatttaagcaaattgtaatcaatgtcagcaaataaactggaaaaagtgcgttgtttctact

SEQ ID NO: 6 - DNA, 1098 bp, Influenza B virus
  source 1..1098: mol_type = other DNA
agcagaagcagaggatttgtttagtcactggcaaacggaaaaaaatggcggacaacatgaccacaacacaaattgaggtgggtccgggagcaaccaatgccactataaactttgaagcaggaattttggagtgctatgaaaggctttcatggcaaagagcccttgactaccctggtcaagaccgcctaaacagactaaagagaaaattagaatcaagaataaagactcacaacaaaagtgagcctgaaagtaaaaggatgtctcttgaagagagaaaagcaattggggtaaaaatgatgaaagtgctcctatttatgaacccatctgctggaattgaagggtttgagccatactgtatgaaaaattcctccaatagcaactgcccaaactgcaattgggccgattaccctccaacatcaggaaagtgccttgatgacatagaagaagaaccggagaatgttgatgacccaactgaaatagtattaagggacatgaacaacaaagatgcaaggcaaaagataaaagaggaagtaaacactcagaaagaagggaagttccgtttgacaataaaaagggatatacgtaatgtgttgtccttgagagtgttggtaaacggaacattcctcaagcaccctaatggatacaagtccttatcaactctgcatagattgaatgcatatgaccagagtggaaggcttgttgctaaacttgttgctactgatgatcttacagtggaggatgaagaagatggccatcggatcctcaactcactcttcgagcgttttaatgaaggacattcaaagccaattcgagcagctgaaactgcggtgggagtcttatcccaatttggtcaagagcaccgattatcaccagaggagggagacaattagactggttacggaagaactttatcttttaagtaaaagaattgatgataacatattgttccacaaaacagtaatagctaacagctccataatagctgacatgattgtatcattatcattattggaaacattgtatgagatgaaggatgtggttgaagtgtacagcaggcagtgcttgtgaatttaaaataaaaatcctcttgttactact

SEQ ID NO: 7 - protein, 583 aa, Influenza B virus
MKAIIVLLMVVTSNADRICTGITSSNSPHVVKTATQGEVNVTGVIPLTTTPTKSHFANLKGTKTRGKLCPNCLNCTDLDVALGRPMCMGTIPSAKASILHEVRPVTSGCFPIMHDRTKIRQLPNLLRGYENIRLSTHNVINAERAPGGPYRLGTSGSCPNVTSRNGFFATMAWAVPRDNKTATNPLTVEVPYICTKGEDQITVWGFHSDDKTQMKNLYGDSNPQKFTSSANGVTTHYVSQIGDFPNQTEDGGLPQSGRIVVDYMVQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMIAIFIVYMVSRDNVSCSICL

SEQ ID NO: 8 - protein, 466 aa, Influenza B virus
MLPSTIQTLTLFLTSGGVLLSLYVSASLSYLLYSDILLKFSPTEITAPKVPLDCANASNVQAVNRSATKGMTLLLSEPEWTYPRLSCQGSTFQKALLISPHRFGETRGNSAPLIIREPFIACGPKECKHFALTHYAAQPGGYYNGTREDRNKLRHLISVKLGKIPTVENSIFHMAAWSGSACHDGREWTYIGVDGPDSNALIKIKYGEAYTDTYHSYANNILRTQESACNCIGGDCYLMITDGSASGISKCRFLKIREGRIIKEIFPTGRVEHTEECTCGFASNKTIECACRDNSYTAKRPFVKLNVETDTAEIRLMCTETYLDTPRPDDGSITGPCESNGDKGRGGIKGGFVHQRMASKIGRWYSRTMSKTERMGMELYVKYDGDPWTDSDALAPSGVMVSMKEPGWYSFGFEIKDKKCDVPCIGIEMVHDGGKKTWHSAATAIYCLMGSGQLLWDTVTGVDMAL

SEQ ID NO: 9 - DNA, 1843 bp, Influenza B virus
  source 1..1843: mol_type = other DNA
tttctaatatccacaaaatgaaggcaataattgtactactcatggtagtaacatccaacgcagatcgaatctgcactgggataacatcttcaaactcacctcatgtggtcaaaacagctactcaaggggaagttaatgtgactggtgtgataccactgacaacaacaccaacaaaatctcattttgcaaatctcaaaggaacaaagaccagagggaaactatgcccaaactgtctcaactgcacagatctggatgtggccttgggcagaccaatgtgtatggggaccataccttcggcaaaagcttcaatactccacgaagtcagacctgttacatccgggtgctttcctataatgcacgacagaacaaaaatcagacagctacccaatcttctcagaggatatgaaaatatcagattatcaacccataacgttatcaacgcagaaagggcaccaggaggaccctacagacttggaacctcaggatcttgccctaacgttaccagtagaaacggattcttcgcaacaatggcttgggctgtcccaagggacaacaaaacagcaacgaatccactaacagtagaagtaccatacatttgcacaaaaggagaagaccaaattactgtttgggggttccattctgatgacaaaacccaaatgaaaaacctctatggagactcaaatcctcaaaagttcacctcatctgccaatggagtaaccacacattatgtttctcagattggtgacttcccaaatcaaacagaagacggagggctaccacaaagcggcagaattgttgttgattacatggtgcaaaaacctgggaaaacaggaacaattgtctatcaaagaggtgttttgttgcctcaaaaggtgtggtgcgcaagtggcaggagcaaggtaataaaagggtccttgcctttaattggtgaagcagattgccttcacgaaaaatacggtggattaaacaaaagcaagccttactacacaggagaacatgcaaaagccataggaaattgcccaatatgggtgaaaacacctttgaagcttgccaatggaaccaaatatagacctcctgcaaaactattaaaggaaaggggtttcttcggagctattgctggtttcttagagggaggatgggaaggaatgattgcaggttggcacggatacacatctcatggagcacatggagtggcagtggcagcagaccttaagagcacgcaagaagccataaacaagataacaaaaaatctcaattctttgagtgagctagaagtaaagaatcttcaaagactaagtggtgccatggatgaactccacaacgaaatactcgagctggatgagaaagtggatgatctcagagctgacacaataagctcgcaaatagagcttgcagtcttgctttccaacgaaggaataataaacagtgaagatgagcatctattggcacttgagagaaaactaaagaaaatgctgggtccctctgctgtagacatagggaatggatgcttcgaaaccaaacacaagtgcaaccagacctgcttagacaggatagctgctggcacctttaatgcaggagaattttctcttcccacttttgattcactgaatattactgctgcatctttaaatgatgatggattggataatcatactatactgctctactactcaactgctgcttctagtttggccgtaacattgatgatagctatttttattgtttatatggtctccagagacaatgtttcttgctccatctgtctataaggaaaattaagccctgtattttcctttattgtagtgcttgtttgcttgttaccattacaaagaaacgttattga

SEQ ID NO: 10 - DNA, 1504 bp, Influenza B virus
  source 1..1504: mol_type = other DNA
aaactgaggcaaataggccaaaaatgaacaatgctaccttcaactatacaaacgttaaccctatttctcacatcagggggagtgttattatcactatatgtgtcagcttcactgtcatacttactgtattcggatatattgctaaaattttcaccaacagaaataactgcaccaaaagtgccattggattgtgcaaacgcatcaaatgttcaggctgtgaaccgttctgcaacaaaagggatgacacttcttctctcagaaccggagtggacataccctcgtttatcttgccagggctcaacctttcagaaagcactcctaattagccctcatagattcggagaaaccagaggaaactcagctcccttgataataagggaaccttttattgcttgtggaccaaaggaatgcaaacactttgctctaacccattatgcagctcaaccagggggatactacaatggaacaagagaggacagaaacaagctgaggcatctgatttcagtcaaattgggcaaaatcccaacagtagaaaactccattttccacatggcagcttggagcgggtccgcatgccatgatggtagagaatggacatatatcggagttgatggccctgacagtaatgcattgatcaaaataaaatatggagaagcatatactgacacataccattcctatgcaaacaacatcctaagaacacaagaaagtgcctgcaattgcatcgggggagattgttatcttatgataactgatggctcagcttcaggaattagtaaatgcagatttcttaagattcgagagggtcgaataataaaagaaatatttccaacaggaagagtagaacatactgaagaatgcacatgcggatttgccagcaataaaaccatagaatgtgcctgtagagataacagttacacagcaaaaagaccctttgtcaaattaaatgtggagactgatacagctgaaataagattgatgtgcacagagacttatttggacacccccagaccagatgatggaagcataacagggccttgcgaatctaatggggacaaagggcgtggaggcatcaagggaggatttgttcatcaaagaatggcatccaagattggaagatggtactctcgaacgatgtctaaaactgaaagaatggggatggaactgtatgtcaagtatgatggagacccatggactgacagtgacgcccttgctcctagtggagtaatggtttcaatgaaagaacctggttggtattcctttggcttcgaaataaaagataagaaatgtgatgtcccctgtattgggatagagatggtacatgatggtggaaaaaagacttggcactcagcagcaacagccatttactgtttaatgggctcaggacaattgctatgggacactgtcacaggtgttgatatggctctgtaatggaggaatggttgagtctgttctaaaccctttgttcctattttgtttgaacaattgtccttactgaacttaa

SEQ ID NO: 11 - DNA, 2396 bp, Influenza B virus; identical to SEQ ID NO: 1
  source 1..2396: mol_type = other DNA

SEQ ID NO: 12 - DNA, 2369 bp, Influenza B virus; identical to SEQ ID NO: 2
  source 1..2369: mol_type = other DNA

SEQ ID NO: 13 - DNA, 2308 bp, Influenza B virus
  source 1..2308: mol_type = other DNA
agcagaagcggtgcgtttgatttgccataatggatacttttattacaagaaacttccagactacaataatacaaaaggccaaaaacacaatggcagaatttagtgaagatcctgaattacaaccagcaatgctattcaacatctgcgtccatctggaggtctgctatgtaataagtgatatgaattttcttgatgaagaaggaaaagcatatacagcattagaaggacaaggaaaagaacaaaacttgagaccacaatatgaagtgattgagggaatgccaagaaacatagcatggatggttcaaagatccttagcccaagagcatgggatagagactccaaggtatctggctgatttgttcgattataaaactaagaggtttatagaagttggaataacaaagggattggctgatgattacttctggaaaaagaaagaaaagctggggaatagcatggaactgatgatattcagctacaatcaagactattcgttaagtaatgaatcctcattggatgaggaaggaaaagggagagtgctaagcagactcacagaacttcaggctgagttaagtctgaaaaatctatggcaagttctcataggagaagaagatattgaaaaaggaattgacttcaaacttggacaaacaatatctaaactaagggatatatctgttccagctggtttctccaattttgaaggaatgaggagctacatagacaatatagatcctaaaggagcaatagagagaaatctagcaaggatgtctcccttagtatcagttacacctaaaaagttgaaatgggaggacctaagaccaatagggcctcacatttacaaccatgagctaccagaagttccatataatgcctttcttctaatgtctgatgaattggggctggctaatatgactgaagggaagtccaagaaaccgaagaccttagccaaagagtgcctagaaaagtactcaacactacgggatcaaactgacccaatattaataatgaaaagcgaaaaagctaatgaacacttcctatggaaactgtggagggactgtgtaaatacaataagtaatgaggaaacaagtaacgaattacagaaaaccaattatgccaagtgggccacaggagatggattaacatatcagaaaataatgaaagaagtagcaatagatgacgaaacaatgtaccaagaagagcccaaaatacctaacaaatgtagagtggctgcttgggttcaaacagagatgaatctattgagcactctgacaagtaaaagggccctggatctgccagaaatagggccagacgtagcacccgtggagcatgtagggagtgaaagaaggaaatactttgttaatgaaatcaactactgtaaggcctctaccgttatgatgaagtatgtgctttttcacacttcattattaaatgaaagcaatgccagtatgggaaaatataaagtaataccaataaccaacagagtagtaaatgaaaagggagaaagttttgacatgctttatggtctagcggttaaagggcaatctcatctgaggggagatactgatgttgtaacagttgtgactttcgaatttagtagtacagatcccagagtggactcaggaaagtggccaaaatatactgtatttagaattggctccttatttgtgagtgggagggaaaaatctgtatacctatattgccgagtgaatggtacaaataagatccaaatgaaatggggaatggaagctagaagatgtctgcttcaatcaatgcaacaaatggaagcaattgttgaacaagaatcatcgatacaaggatatgacatgaccaaagcttgtttcaagggagacagagtgaatagtccaaaaactttcagtattgggactcaagaaggaaaactagtgaaaggatcctttgggaaagcactaagagtaatattcaccaaatgtttgatgcactatgtatttggaaatgcccaattggaggggtttagtgccgaatctaggagacttctactgttaattcaggcattaaaagacagaaagggcccttgggtattcgacttagagggaatgtattctggaatagaagaatgtattagtaacaacccttgggtaatacagagtgcatactggtttaatgaatggttgggctttgaaaaagaggggagtaaagtattagaatcagtagatgaaataatggatgaatgaaagaagggcatagtgctcaatttggtactattttgttcattatgtatctaaacatccaatataaagaattgagaattaaaaatgcacgtgtttctact

SEQ ID NO: 14 - DNA, 1843 bp, Influenza B virus
  source 1..1843: mol_type = other DNA
agcagaagcacagcattttcttgtgaacttcaagtaccaacaaaaaactgaaaatcaaaatgtccaacatggacattgacggcatcaacactggaataattgacaaaacaccagaagaaataacttccggaaccagtggggcaaccagaccaatcatcagaccagcaacccttgcctcaccaagcaacaaacgaaccagaaacccatccccggaaagggcaaccacaagcagtgaagctgatgtcggaaggaaaacccaaaagaaacaaactccgacagagataaagaagagcgtctacaatatggtagtgaaactgggtgaattctacaaccagatgatggtcaaagctggactcaacgatgacatggagagaaacctaatccaaaatgcacatgctgtggaaagaattctattggctgctactgatgacaagaaaactgaattccaaaagaaaaagaatgccagagacgtcaaagaagggaaagaagaaatagaccataacaaaacaggaggcaccttttacaagatggtaagagatgataaaaccatctacttcagccctataagaattacctttttaaaagaagaggtgaaaacaatgtacaaaaccaccatggggagtgacggtttcagtggactaaatcacatcatgattgggcattcacagatgaacgatgtctgtttccaaagatcaaaggcactaaaaagagttggacttgacccttcattaatcagtacttttgcaggaagcacactccccagaagatcaggtacaactggtgttgcgaccaaaggaggtggaactttagtggcagaagccattcgatttataggaagagcaatggcagacagagggctattgagagacatcagagccaagacggcctatgaaaagattcttctgaatctgaaaaacaagtgctctgcgccccaacaaaaggctctggttgatcaagtgatcggaagtagaaatccagggattgcagacatagaagatctcaccctgcttgctcgaagtatggtcgttgttaggccctctgtagcaagcaaagtggtgcttcccataagcatctatgctaaaatacctcaactggggttcaacgttgaagaatactctatggttgggtatgaagccatggctctttataatatggcaacacctgtttccatattaagaatgggagacgatgcaaaagataaatcacaattattcttcatgtcttgctttggagctgcctatgaagacctaagagttctgtctgcactaacaggcacggaattcaagcctaggtcagcattaaagtgcaaaggtttccacgttccagcaaaggagcaagtggaaggaatgggggcagctctgatgtccatcaagctccagttttgggctccaatgaccagatctggggggaatgaagtaggtggagacggagggtctggtcaaataagttgcagccccgtgtttgcagtagaaagacctattgctctaagcaagcaagctgtaagaagaatgctgtcaatgaatattgagggacgtgatgcagatgtcaaaggaaatctactcaagatgatgaatgattcaatggctaagaaaaccaatggaaatgctttcattgggaagaaaatgttccaaatatcagacaaaaacaaaaccaatcccgttgagattccaattaagcagaccatccccagtttcttctttgggagggacacagcagaggattatgatgacctcgattattaaagcaacaaaatagacactatggctgtgattgtttcagtacgtttggaatgtgggtgtttactcttattgaaataaatgtaaaaaatgctgttgtttctact

SEQ ID NO: 15 - DNA, 1843 bp, Influenza B virus
  source 1..1843: mol_type = other DNA
agcagaagcacagcattttcttgtgaacttcaagtaccaacaaaaaactgaaaatcaaaatgtccaacatggacattgacggcatcaacactggaataattgacaaaacaccagaagaaataacttccggaaccagtggggcaaccagaccaatcatcagaccagcaacccttgcctcaccaagcaacaaacgaaccagaaacccatccccggaaagggcaaccacaagcagtgaagctgatgtcggaaggaaaacccaaaagaaacaaactccgacagagataaagaagagcgtctacaatatggtagtgaaactgggtgaattctacaaccagatgatggtcaaagctggactcaacgatgacatggagagaaacctaatccaaaatgcacatgctgtggaaagaattctattggctgctactgatgacaagaaaactgaattccaaaagaaaaagaatgccagagacgtcaaagaagggaaagaagaaatagaccataacaaaacaggaggcaccttttacaagatggtaagagatgataaaaccatctacttcagccctataagaattacctttttaaaagaagaggtgaaaacaatgtacaaaaccaccatggggagtgacggtttcagtggactaaatcacatcatgattgggcattcacagacgaacgatgtctgtttccaaagatcaaaggcactaaaaagagttggacttgacccttcattaatcagtacttttgcaggaagcacactccccagaagatcaggtacaactggtgttgcgaccaaaggaggtggaactttagtggcagaagccattcgatttataggaagagcaatggcagacagagggctattgagagacatcagagccaagacggcctatgaaaagattcttctgaatctgaaaaacaagtgctctgcgccccaacaaaaggctctggttgatcaagtgatcggaagtagaaatccagggattgcagacatagaagatctcaccctgcttgctcgaagtatggtcgttgttaggccctctgtagcaagcaaagtggtgcttcccataagcatctatgctaaaatacctcaactggggttcaacgttgaagaatactctatggttgggtatgaagccatggctctttataatatggcaacacctgtttccatattaagaatgggagacgatgcaaaagataaatcacaattattcttcatgtcttgctttggagctgcctatgaagacctaagagttctgtctgcactaacaggcacggaattcaagcctaggtcagcattaaagtgcaaaggtttccacgttccagcaaaggagcaagtggaaggaatgggggcagctctgatgtccatcaagctccagttttgggctccaatgaccagatctggggggaatgaagtaggtggagacggagggtctggtcaaataagttgcagccccgtgtttgcagtagaaagacctattgctctaagcaagcaagctgtaagaagaatgctgtcaatgaatattgagggacgtgatgcagatgtcaaaggaaatctactcaagatgatgaatgattcaatggctaagaaaaccaatggaaatgctttcattgggaagaaaatgttccaaatatcagacaaaaacaaaaccaatcccgttgagattccaattaagcagaccatccccagtttcttctttgggagggacacagcagaggattatgatgacctcgattattaaagcaacaaaatagacactatggctgtgattgtttcagtacgtttggaatgtgggtgtttactcttattgaaataaatgtaaaaaatgctgttgtttctact

SEQ ID NO: 16 - DNA, 1190 bp, Influenza B virus
  source 1..1190: mol_type = other DNA
agcagaagcacgcactttcttaaaatgtcgctgtttggagacacaattgcctacctgctttcactaacagaagatggagaaggcaaagcagaactagcggaaaaattacactgttggttcggtgggaaagaattcgatctagactctgctttggaatggataaaaaacaaaagatgcctaactgatatacaaaaagcactaattggtgcctctatctgctttttgaaacccaaagaccaagaaagaaaaagaaaattcatcacagagcccctgtcaggaatgggaacaacagcaacaaaaaagaaaggcctgattctagctgaaagaaaaatgagaagatgtgtgagttttcatgaggcatttgaaatagcagaaggccatgaaagctcagcactactatattgtctcatggtcatgtacctgaaccctggaaattattcaatgcaagtaaaactaggaacgctctgtgctttatgcgagaaacaagcatcacattcacacagagctcatagcagagcagcaagatcttcagtgcctggagtgaggcgagaaatgcaaatggtttcagctatgaacacagcaaaaacaatgaatggaatggggaagggagaagacgtccaaaaactggcagaagagctgcaaagcaacattggagtattgagatctctgggggcaagtcaaaagaatggagaaggaattgcaaaggatgtaatggaagtgctaaagcagagctccatgggaaattcagctcttgtgaagaaatacctataatgctcgagccatttcagattctttcaatttgctctttcattttatcggctctccatttcatgggctggacaatagggcatttaaatcaaataaaaagaggagtaaacctaaaaatacgaataagaaatccaaataaagagacaataaatagagaggtatcaattttgagacacagttaccaaaaagaaatccaagccaaagaaacaataaaggaagtactctctgacaacatggagagattgagtgaccacatagtaattgaggggctttctgctgaagagataataaaaatgggtgaaacagttttggaggtagaagaattgcattaaacccaattttcaccgtatttcttactatgcatttaagcaaattgtaatcaatgtcagcaaataaactggaaaaagtgcgttgtttctact

SEQ ID NO: 17 - DNA, 1190 bp, Influenza B virus
  source 1..1190: mol_type = other DNA
agcagaagcacgcactttcttaaaatgtcgctgtttggagacacaattgcctacctgctttcactaacagaagatggagaaggcaaagcagaactagcggaaaaattacactgttggttcggtgggaaagaattcgatctagactctgctttggaatggataaaaaacaaaagatgcctaactgatatacaaaaagcactaattggtgcctctatctgctttttgaaacccaaagaccaagaaagaaaaagaagattcatcacagagcccctgtcaggaacgggaacaacagcaacaaaaaagaaaggcctgattctagctgaaagaaaaatgagaagatgtgtgagttttcatgaggcatttgaaatagcagaaggccatgaaagctcagcactactatattgtctcatggtcatgtacctgaaccctggaaattattcaatgcaagtaaaactaggaacgctctgtgctttatgcgagaaacaagcatcacattcacacagagctcatagcagagcagcaagatcttcagtgcctggagtgaggcgagaaatgcaaatggtttcagctatgaacacagcaaaaacaatgaatggaatggggaagggagaagacgtccaaaaactggcagaagagctgcaaagcaacattggagtattgagatctctgggggcaagtcaaaagaatggagaaggaattgcaaaggatgtaatggaagtgctaaagcagagctccatgggaaattcagctcttgtgaagaaatacctataatgctcgagccatttcagattctttcaatttgctctttcattttatcggctctccatttcatgggctggacaatagggcatttaaatcaaataaaaagaggagtaaacctaaaaatacgaataagaaatccaaataaagagacaataaatagagaggtatcaattttgagacacagttaccaaaaagaaatccaagccaaagaaacaataaaggaagtactctctgacaacatggagagattgagtgaccacatagtaattgaggggctttctgctgaagagataataaaaatgggtgaaacagttttggaggtagaagaattgcattaaacccaattttcaccgtatttcttactatgcatttaagcaaattgtaatcaatgtcagcaaataaactggaaaaagtgcgttgtttctact

SEQ ID NO: 18 - DNA, 1098 bp, Influenza B virus
  source 1..1098: mol_type = other DNA
agcagaagcagaggatttgtttagtcactggcaaacgggaaaaaatggcggacaacatgaccacaacacaaattgaggtgggtccgggagcaaccaatgccactataaactttgaagcaggaattttggagtgctatgaaaggctttcatggcaaagagcccttgactaccctggtcaagaccgcctaaacagactaaagagaaaattagaatcaagaataaagactcacaacaaaagtgagcctgaaagtaaaaggatgtctcttgaagagagaaaagcaattggggtaaaaatgatgaaagtgctcctatttatgaacccatctgctggaattgaagggtttgagccatactgtatgaaaaattcctccaatagcaactgcccaaactgcaattgggccgattaccctccaacatcaggaaagtgccttgatgacatagaagaagaaccggagaatgttgatgacccaactgaaatagtattaagggacatgaacaacaaagatgcaaggcaaaagataaaagaggaagtaaacactcagaaagaagggaagttccgtttgacaatacaaagggatatacgtaatgtgttgtccttgagagtgttggtaaacggaacattcctcaagcaccctaatggatacaagtccttatcaactctgcatagattgaatgcatatgaccagagtggaaggcttgttgctaaacttgttgctactgatgatcttacagtggaggatgaagaagatggccatcggatcctcaactcactcttcgagcgttttaatgaaggacattcaaagccaattcgagcagctgaaactgcggtgggagtcttatcccaatttggtcaagagcaccgattatcaccagaggagggagacaattagactggttacggaagaactttatcttttaagtaaaagaattgatgataacatattgttccacaaaacagtaatagctaacagctccataatagctgacatgattgtatcattatcattattggaaacattgtatgagatgaaggatgtggttgaagtgtacagcaggcagtgcttgtgaatttaaaataaaaatcctcttgttactact

SEQ ID NO: 19 - DNA, 1099 bp, Influenza B virus
  source 1..1099: mol_type = other DNA
agcagaagcagaggatttgtttagtcactggcaaacgggaaaaaaatggcggacaacatgaccacaacacaaattgaggtgggtccgggagcaaccaatgccactataaactttgaagcaggaattttggagtgctatgaaaggctttcatggcaaagagcccttgactaccctggtcaagaccgcctaaacagactaaagagaaaattagaatcaagaataaagactcacaacaaaagtgagcctgaaagtaaaaggatgtctcttgaagagagaaaagcaattggggtaaaaatgatgaaagtgctcctatttatgaacccatctgctggaattgaagggtttgagccatactgtatgaaaaattcctccaatagcaactgcccaaactgcaattgggccgattaccctccaacatcaggaaagtgccttgatgacatagaagaagaaccggagaatgttgatgacccaactgaaatagtattaagggacatgaacaacaaagatgcaaggcaaaagataaaagaggaagtaaacactcagaaagaagggaagttccgtttgacaataaaaagggatatacgtaatgtgttgtccttgagagtgttggtaaacggaacattcctcaagcaccctaatggatacaagtccttatcaactctgcatagattgaatgcatatgaccagagtggaaggcttgttgctaaacttgttgctactgatgatcttacagtggaggatgaagaagatggccatcggatcctcaactcactcttcgagcgttttaatgaaggacattcaaagccaattcgagcagctgaaactgcggtgggagtcttatcccaatttggtcaagagcaccgattatcaccagaggagggagacaattagactggttacggaagaactttatcttttaagtaaaagaattgatgataacatattgttccacaaaacagtaatagctaacagctccataatagctgacatgattgtatcattatcattattggaaacattgtatgagatgaaggatgtggttgaagtgtacagcaggcagtgcttgtgaatttaaaataaaaatcctcttgttactact

(19) United States
(12) Patent Application Publication
(10) Pub. No.: US 2026/0041756 A1
(43) Pub. Date: Feb. 12, 2026

(54) INFLUENZA B VIRUS REPLICATION FOR VACCINE DEVELOPMENT

(71) Applicant: Wisconsin Alumni Research Foundation (WARF), Madison, WI (US)

(72) Inventors: Yoshihiro Kawaoka, Middleton, WI (US); Gabriele Neumann, Madison, WI (US); Jihui Ping, Nanjing (CN)

(21) Appl. No.: 19/240,777

(22) Filed: Jun. 17, 2025

Related U.S. Application Data
(63) Continuation of application No. 17/546,835, filed on Dec. 9, 2021, now Pat. No. 12,364,748.
Application No. 17/546,835 is a continuation of application No. 15/436,245, filed on Feb. 17, 2017, now Pat. No. 11,197,925.
(60) Provisional application No. 62/297,400, filed on Feb. 19, 2016.

(51) Int. Cl. A61K 39/145; A61K 39/00; A61K 39/12; C12N 7/00; C12N 15/86

(52) U.S. Cl. CPC A61K 39/145; A61K 39/12; C12N 7/00; C12N 15/86; A61K 2039/5258; C12N 2760/16023; C12N 2760/16071; C12N 2760/16221; C12N 2760/16222; C12N 2760/16234; C12N 2760/16251

(57) ABSTRACT

The invention provides a composition useful to prepare high titer influenza B viruses, e.g., in the absence of helper virus, which includes internal genes from an influenza B virus vaccine strain or isolate, e.g., one that is safe in humans, for instance, one that does not result in significant disease, that confer enhanced growth in cells in culture, such as MDCK cells, or in eggs.

### CROSS-REFERENCE TO RELATED APPLICATIONS

This application is a continuation of U.S. application Ser. No. 17/546,835, filed Dec. 9, 2021, which is a continuation of U.S. application Ser. No. 15/436,245, filed Feb. 17, 2017, which claims the benefit of the filing date of U.S. application Ser. No. 62/297,400, filed on Feb. 19, 2016, the disclosure of which is incorporated by reference herein.

### STATEMENT OF GOVERNMENT RIGHTS

This invention was made with government support under HHSN272201400008C awarded by the National Institutes of Health. The government has certain rights in the invention.

### INCORPORATION BY REFERENCE OF SEQUENCE LISTING

This application contains a Sequence Listing which has been submitted electronically in ST26 format and hereby incorporated by reference in its entirety. Said ST26 file, created on Oct. 28, 2025, is named 800097US3.xml and is 49,152 bytes in size.

### BACKGROUND

Influenza B viruses are a major cause of respiratory disease in humans. The segmented nature of the influenza virus genome allows for reassortment of segments during virus replication in cells infected with two or more influenza viruses. The reassortment of segments, combined with genetic mutation and drift, can give rise to a myriad of divergent strains of influenza virus overtime. The new strains exhibit antigenic variation in their hemagglutinin (HA) and/or neuraminidase (NA) proteins, and in particular the gene coding for the HA protein has a high rate of variability. The predominant current practice for the prevention of flu is vaccination. As the influenza HA protein is the major target antigen for the protective immune responses of a host to the virus and is highly variable, the isolation of influenza virus and the identification and characterization of the HA antigen in viruses associated with recent outbreaks is important for vaccine production. Based on prevalence and prediction, a vaccine is designed to stimulate a protective immune response against the predominant and expected influenza virus strains (Park et al., 2004).

There are three general types of influenza viruses, Type A, Type B and Type C, which are defined by the absence of serological crossreactivity between their internal proteins. Influenza Type B viruses are further classified into two lineages based on antigenic and genetic differences of the glycoprotein HA.

The burden of human infections with influenza A and B viruses is substantial, and the impact of influenza B virus infections can exceed that of influenza A virus infections in some seasons. Over the past few decades, viruses of two influenza B virus lineages (Victoria and Yamagata) have circulated in humans, and both lineages are now represented in influenza vaccines, as recommended by the World Health Organization. Influenza B virus vaccines for humans have been available for more than half a century, yet no systematic efforts have been undertaken to develop high-yield candidates. On the basis of their antigenic properties, influenza viruses are divided into three types (influenza A, B, and C); however, only type A and B influenza viruses cause human health concerns. Influenza A viruses are further divided into 18 hemagglutinin (HA, the major viral antigen) and 11 neuraminidase (NA, the second viral antigen) subtypes that are referred to as H1-H18 and N1-N11, respectively. Influenza A viruses are responsible for annual epidemics (caused by antigenic escape variants possessing point mutations in the antigenic epitopes of HA) and occasional pandemics. Pandemics are caused by avian or avian/human/swine reassortant influenza viruses that encode an HA protein to which humans lack protective immune responses. The epidemiology of influenza B viruses differs from that of influenza A viruses. Influenza B viruses primarily circulate in humans and do not cause pandemics. Nevertheless, the impact of influenza B virus infections on influenza-related morbidity and mortality is substantial and has exceeded that of influenza A viruses in some seasons (Paul-Glezen et al., 2013; Tafalla et al., 2016; van de Sandt et al., 2015). Until 1983, only one influenza B virus lineage was circulating in humans. Since then, two lineages (Victoria, named after B/Victoria/2/1987, and Yamagata, named after B/Yamagata/16/1988) can be distinguished genetically and antigenically on the basis of their HA (Paul-Glezen et al., 2013; van de Sandt et al., 2015; Rota et al., 1990). Until 2000, one of these two lineages tended to dominate each season; however, since 2001 both influenza B virus lineages have been cocirculating in human populations each year (Belshe, 2010; Belshe et al., 2010).

Until recently, most influenza vaccines were trivalent: that is, they were comprised of influenza A strains of the H1N1 and H3N2 subtypes, and an influenza B virus strain. Two studies demonstrated that the recommended influenza B vaccine strain matched the dominant strain of the particular influenza season only half the time (Belshe, 2010; Ambrose et al., 2010). On the basis of these findings and the continuing cocirculation of Yamagata- and Victoria-lineage viruses, in 2012 theWorld Health Organization (WHO) recommended including influenza B viruses of both lineages in human influenza vaccines. Accordingly, most seasonal influenza vaccines are now quadrivalent.

Many influenza vaccines are generated by combining the HA and NA viral RNA (vRNA) segments of WHO-recommended vaccine strains with the remaining six vRNA segments of a “backbone” strain. For live attenuated influenza A and B vaccine viruses, virus backbones were developed in the 1960s (Maassab, 1969). Many inactivated influenza A virus vaccines are based on the A/Puerto Rico/8/34 (H1 N1; PR8) virus backbone, which was selected because of its efficient replication in embryonated chicken eggs. For inactivated influenza B vaccines, the B/Lee/40, B/Panama/45/90, or wild-type strains have been used as backbones (www.who.int/influenza/vaccines/virus/recommendations/summary_b_vic_cvv_nh1516.pdf).

### SUMMARY

The present invention relates to several mutations in the ‘internal’ genes of influenza B virus, as well as several mutations in the viral glycoproteins HA and NA of influenza B viruses, that enhance viral titers and/or HA yields in cultured cells and may also enhance viral titers and/or HA yields in embryonated chicken eggs. The exemplary reassortant or recombinant parental influenza B viruses represent the two major influenza B virus lineages (i.e., the ‘B/Victoria’ and ‘B/Yamagata’ lineages). Virus libraries were generated for each lineage, which libraries were then passaged in selected cells, and mutations were identified that enhanced viral growth. The use of one or more of these mutations in vaccine virus master strains (where the internal viral genes, the “backbone,” are used with selected HA and NA, e.g., those of circulating strains or predicted to be circulating strains), result in higher virus titers in virus cultured in cells in vitro and/or embryonated chicken eggs, allowing more efficient influenza B virus growth, and more rapid and cost-effective vaccine production.

Several strategies may be employed (including random mutagenesis and the comprehensive testing of growth-enhancing mutations) to develop influenza B viruses, e.g., based on reassortant or recombinant B/Yamagata- and B/Victoria-viruses, with enhanced properties, e.g., viruses that replicate to high titers, e.g., 10³ PFU/mL or more, e.g., 5×10⁸, 10⁹, 5×10⁹ or 10¹⁰ PFU/mL in cultured cells and/or embryonated chicken eggs. As discussed herein, a number of growth-enhancing mutations (both amino acid and non-coding nucleotide substitutions) were identified that increase the yield of influenza B viruses. Individual growth-enhancing amino acid residues in an influenza B virus polypeptide or in non-coding nucleotide sequence(s) in an influenza B virus segment, may be combined with one or more other growth-enhancing residues in the same influenza virus polypeptide or non-coding nucleotides in the same viral segment(s), or with one or more other growth-enhancing residues and/or nucleotide substitution(s) in other influenza virus polypeptide(s) or viral segment(s), respectively, e.g., growth-enhancing nucleotides in promoter sequences or in nucleotides between promoter sequences and an open reading frame. In particular, virus libraries possessing random mutations in the six “internal” influenza B viral RNA segments (those not encoding the major viral antigens, hemagglutinin (HA) and neuraminidase NA)) were screened for mutants that confer efficient replication. Candidate viruses that supported high yield in cell culture were tested with the HA and NA genes of eight different viruses of the Victoria and Yamagata lineages. Combinations of mutations that increased the titers of candidate vaccine viruses in mammalian cells used for human influenza vaccine virus propagation were identified and used in embryonated chicken eggs, the most common propagation system for influenza viruses, were identified. These influenza B virus vaccine backbones can be used for improved vaccine virus production.

For example, one or more growth-enhancing residues in a NP protein, for instance, 1, 2, 3, or 4 or more, growth-enhancing residues in NP, 1, 2, 3, or 4 or more, growth-enhancing residues in a M protein (such as 1, 2, 3, or 4 growth-enhancing residues in BM2 or 1, 2, 3, or 4 growth-enhancing residues in M1), 1, 2, 3, or 4 or more growth-enhancing residues in PA, or 1, 2, 3, or 4 growth-enhancing residues in NS1, or growth-enhancing nucleotides in viral non-coding sequences of NP, PA, NS, or in other viral segments, may be combined when preparing influenza B viruses, e.g., for a vaccine, to enhance viral titers. In one embodiment, growth-enhancing nucleotides in non-coding sequences may be introduced to a viral segment, or when present in a viral segment may be selected for inclusion in an influenza B virus. In one embodiment, one or more, e.g., 1, 2, 3, 4 or 5 or more growth-enhancing residues in HA and/or in NA may be introduced into, or when present in a HA or NA selected for inclusion in, a HA viral segment or a NA viral segment in an influenza virus. In one embodiment, the one or more growth-enhancing residues may enhance viral growth by at least 1.2, 2, 2.8, 4, 3, 5, 6, 8, 10, 100, or 200 fold or more.

In one embodiment, this disclosure provides isolated recombinant, e.g., reassortant, influenza B viruses with selected amino acid residues at one or more specified positions (including those described herein) in one or more viral segments for PA, PB1, PB2, NP, M (encoding M1 and BM2 proteins), and/or NS (encoding NS1 and NS2 proteins), e.g., in selected amino acid residues at specified positions of, for example, M1 and BM2; M1, BM2, and NS1; NP; M1 and NS1; NP, M1 and BM2; NP and M1; NP, M1 and NS1: BM2 and NS1; BM2, NS1 and PA; M1, BM2 and PA; M1, BM2, NP and PA; and optionally also including growth-enhancing non-coding nucleotide substitution(s), and in one embodiment, including HA and NA genes/proteins of interest, e.g., from annual and pandemic strains, or HA and NA viral segments with selected amino acid residues described herein, which viruses are produced more efficiently and cost-effectively via cell culture (in MDCK or Vero cells) or in embryonated chicken eggs.

In one embodiment, the reassortant or recombinant influenza B virus has an amino acid residue at position 28, 40, 51, 52, 57, 204, and/or 343, in NP, and/or a nucleotide other than c at position 500 in NP vRNA, or any combination thereof, that results in enhanced growth in cells including MDCK cells, Vero cells and/or eggs relative to a corresponding virus with, for instance, an alanine, proline, proline, glutamic acid, serine, methionine or proline at position 28, 40, 51, 52, 57, 204 and 343, respectively, in NP, i.e., the residue at position 28, 40, 51, 52, 57, 204 or 343, respectively, in the NP segment in the recombinant influenza B virus is not an alanine, proline, proline, glutamic acid, serine, methionine or proline but is a residue that is correlated with enhanced replication in MDCK cells, Vero cells or eggs. The recombinant virus may also optionally include other selected amino acid residues at one or more specified positions in one or more of M1, BM2, PA, PB2, and/or NS1, such as those described herein, and optionally PB1. In one embodiment, the recombinant influenza B virus has an amino acid residue at position 28, 40, 51, 52, 57, 204, and/or 343 in NP that results in enhanced interaction with one or more host proteins in MDCK cells, Vero cells or eggs relative to a corresponding virus with, for instance, alanine, proline, proline, glutamic acid, serine, methionine or proline at position 28, 40, 51, 52, 57, 204, and/or 343, respectively, in NP. In one embodiment, the recombinant influenza B virus has growth-enhancing residues in NP including but not limited to a residue other than alanine at position 28, other than proline at position 40, other than proline at position 51, other than glutamic acid at position 52, other than serine at position 57, other than methionine at position 204, and/or other than proline at position 343, and/or a nucleotide other than g at nucleotide position 1795 (italics indicates a nucleotide; position is relative to positive sense cRNA), or any combination thereof. In one embodiment, the recombinant influenza B virus has threonine at position 28, serine at position 40, glutamine at position 51, lysine at position 52, glycine at position 57, threonine at position 214, and/or threonine at position 343 in NP, nucleotide a at nucleotide position 1795, and/or nucleotide tat nucleotide position 500 in NP vRNA, or any combination thereof, as well as optionally selected amino acid residues at one or more specified positions in M, PA, PB1, PB2, and/or NS viral segments.

In one embodiment, the reassortant or recombinant influenza B virus has an amino acid residue at position 34, 54, 77, 86, and/or 97 in M1 that results in enhanced growth in cells including MDCK cells, Vero cells or eggs relative to a corresponding virus with, for instance, glycine, aspartic acid, arginine, methionine or isoleucine at position 34, 54, 77, 86, or 97, respectively, in M1, i.e., the residue at position 34, 54, 77, 86, or 97, respectively, in M1 in the M segment in the recombinant influenza virus is not glycine, aspartic acid, arginine, methionine or isoleucine but is a residue that is correlated with enhanced replication in MDCK cells, Vero cells or eggs. The recombinant virus may also optionally include selected amino acid residues at one or more specified positions PA, BM2, PB2, NP, and/or NS1, such as those described herein, and optionally PB1. In one embodiment, the recombinant influenza B virus has an amino acid residue at position 34, 54, 77, 86, and/or 97 in M1 that results in enhanced interaction with one or more host proteins in MDCK cells, Vero cells or eggs relative to a corresponding virus with, for instance, glycine, aspartic acid, arginine, methionine or isoleucine at position 34, 54, 77, 86, or 97, respectively, in M1. In one embodiment, the recombinant influenza B virus has a valine or asparagine, glycine, lysine, threonine or asparagine at position 34, 54, 77, 86, or 97, respectively, in M1 as well as optionally selected amino acid residues at one or more specified positions NP, PA, PB1, PB2, and/or NS.

In one embodiment, the reassortant or recombinant influenza B virus has an amino acid residue at position 26, position 27, position 58, or position 80 in BM2 that results in enhanced growth in cells including MDCK cells, Vero cells or eggs relative to a corresponding virus with, for instance, glycine, histidine, histidine or arginine, at residue 26, 27, 58 or 80, respectively, in BM2. The recombinant virus may also optionally include as selected amino acid residues at one or more specified positions NS1, PA, NP, PB2, and/or M1 which are described herein. In one embodiment, the residue in BM2 at position 26 is arginine, at position 27 is arginine, position 58 is arginine, or position 80 is glycine.

In one embodiment, the reassortant or recombinant influenza B virus has an amino acid residue other than tyrosine at position 42, other than methionine at position 117, other than lysine at position 176, and/or other than serine at position 252, a nucleotide other than a at position 39, a nucleotide insertion after position 38, or any combination thereof, in NS1 that results in enhanced growth in cells including MOCK cells, Vero cells or eggs relative to a corresponding virus with, for instance, tyrosine at position 42, methionine at position 117, lysine at position 176, serine at position 252, or an a at position 39. The recombinant virus may also optionally include selected amino acid residues at one or more specified positions PA, PB2, BM2, NP, and/or M1, e.g., those which are described herein, and optionally PB1. In one embodiment, the recombinant influenza virus has asparagine at position 42, tyrosine at position 117, glutamine at position 176, threonine at position 252, a g at nucleotide position 39, an additional g after nucleotide position 38, or any combination thereof, in NS1, and optionally selected amino acid residues at one or more specified positions PA, PB2, BM2, NP, and/or M1 which are described herein.

In one embodiment, the reassortant or recombinant influenza B virus has an amino acid residue in PA other than tyrosine at position 387, other than valine at position 434, other than aspartic acid at position 494, other than threonine at position 524, and/or a nucleotide other than a at position 2272, a nucleotide other than g at position 2213, a nucleotide other than a at position 1406, and/or a nucleotide other than c at position 1445 in PA vRNA, or any combination thereof, that results in enhanced growth in cells including MOCK cells, Vero cells or eggs relative to a corresponding virus with, for instance, tyrosine at position 387, valine at position 434, aspartic acid at position 494, threonine at position 524, and/or a nucleotide a at position 2272, g at position 2213, a at position 1406, or c at position 1445, or any combination thereof. The recombinant virus may also optionally include selected amino acid residues at one or more specified positions NS1, BM2, NP, PB2, and/or M1, e.g., those which are described herein, and optionally PB1. In one embodiment, the recombinant influenza virus has histidine at position 387, alanine at position 434, or asparagine at position 494, alanine at position 534 in PA, and/or t at position 2272, a at position 2213, g at position 1406, and/or t at position 1445 in PA vRNA, or any combination thereof.

In one embodiment, the reassortant or recombinant influenza B virus has an amino acid residue in PB2 other than asparagine at position 16 that results in enhanced growth in cells including MOCK cells, Vero cells or eggs relative to a corresponding virus with, for instance, asparagine at position 16. The recombinant virus may also optionally include selected amino acid residues at one or more specified positions PA, NS1, BM2, NP, and/or M1, e.g., those which are described herein, and optionally PB1. In one embodiment, the recombinant influenza virus has serine at position 16 in PB2.

In one embodiment, the reassortant or recombinant influenza B virus has an amino acid residue in HA1 other than threonine at position 34, other than arginine at position 98, other than lysine at position 129, other than asparagine at position 168, other than asparagine at position 194, and/or other than threonine at position 196, and/or in HA2 a residue other than lysine at position 39, other than serine at position 56, other than lysine at position 61, or other than aspartic acid at position 112, or any combination thereof, that results in enhanced growth in cells including MDCK cells, Vero cells or eggs relative to a corresponding virus with, for instance, in HA1 threonine at position 34, arginine at position 98, lysine at position 129, asparagine at position 168, asparagine at position 194, and/or threonine at position 196, and/or in HA2 lysine at position 39, serine at position 56, lysine at position 61, aspartic acid at position 112. The recombinant virus may also include selected amino acid residues at one or more specified positions PA, BM2, PB2, NS1, NP, and/or M1, e.g., those which are described herein, and/or PB1. In one embodiment, the recombinant influenza virus has isoleucine at position, 34, glutamic acid at position 129, glutamic acid or aspartic acid at position 168, proline, alanine, isoleucine or asparagine at position 196, lysine at position 98, aspartic acid at position 194, glycine at position 39 (in HA2), glycine at position 56 (in HA2), asparagine at position 51 (in HA2), or glutamic acid at position 112 (in HA2), or any combination thereof.

In one embodiment, the reassortant or recombinant influenza B virus has an amino acid residue other than threonine (T) at position 76, other than arginine (R) at position 102, other than glutamic acid (E) at position 105, other than proline (P) at position 139, other than asparagine (N) at position 169, other than glycine (G) at position 434, other than threonine at position 436, and/or other than aspartic acid (D) at position 457, or any combination thereof, in NA that results in enhanced growth in cells including MDCK cells, Vero cells or eggs relative to a corresponding virus with, for instance, T at position 76, R at position 102, E at position 105, P at position 139, N at position 169, G at position 434, T at position 436, and/or D at position 457 in NA, which recombinant virus may also optionally include selected amino acid residues at one or more specified positions PA, PB2, BM2, NP, NS1, and/or M1, e.g., those which are described herein, PB1. In one embodiment, the recombinant influenza B virus has methionine (M) at position 76, lysine (K) at position 102, lysine (K) at position 105, serine (S) at position 139, threonine (T) at position 169, glutamic acid (E) at position 434, methionine (M) at position 436, and/or asparagine (N) at position 457, or any combination thereof.

In one embodiment, the invention provides an isolated recombinant reassortant influenza virus having six “internal” gene segments from a vaccine influenza virus with two or more of the selected amino acid residues at specified positions described herein, and a NA gene segment selected from a first influenza virus isolate, and a HA gene segment from the same isolate or a different isolate.

In one embodiment, the influenza virus of the invention is a recombinant influenza B virus having a particular amino acid residue at specified positions in one, two, three or more of PA, PB1, PB2, NP, M1 and/or NS1 and having an amino acid sequence with at least 80%, e.g., 90%, 92%, 95%, 97%, 98%, or 99%, including any integer between 80 and 99, contiguous amino acid sequence identity to a corresponding polypeptide encoded by one of SEQ ID Nos. 1-6 (the internal genes of B/Yamagata/1/73), such as a polypeptide with other than A at position 28, other than P at position 40, other than P at position 51, other than E at position 52, other than S at position 57, other than M at position 204, and/or other than P at position 343, in NP and/or g at position 1795 or t at position 500 in NP vRNA, or any combination thereof; residue other than G at position 34, other than D at position 54, other than R at position 77, other than M at position 86, other than I at position 97, or any combination thereof, in M1; other than H at position 58, other than R at position 80, other than H at position 27, other than G at position 26, in BM2, e.g. R at position 58, G at position 80, R at position 27, and/or R at position 26 in BM2; other than Y at position 42, other than M at position 117, other than K at position 176, and/or other than S at position 252, in SN1 and/or a39g, an additional g after 38, in NS1 vRNA, or any combination thereof; other than N at position 16 in PB2; and/or other than Y at position 387, other than V at position 434, other than D at position 494, other than T at position 524, in PA and/or a2272t, g2213a, a1406g, and/or c1445t, in PA vRNA, or any combination thereof. The residue other than the specified residue may be a conservative substitution. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine and tryptophan; a group of amino acids having basic side chains is lysine, arginine and histidine; and a group of amino acids having sulfur-containing side chain is cysteine and methionine. In one embodiment, conservative amino acid substitution groups are: threonine-valine-leucine-isoleucine-alanine; phenylalanine-tyrosine; lysine-arginine; alanine-valine; glutamic-aspartic; and asparagine-glutamine. Non-conservative substitutions are also envisioned.

In one embodiment, the influenza B virus of the invention is a recombinant influenza B virus having a particular amino acid residue at specified positions in one, two, three or more of PA, NS1, M or NP which polypeptides have an amino acid sequence with at least 80%, e.g., 90%, 92%, 95%, 97%, 98%, or 99%, including any integer between 80 and 99, contiguous amino acid sequence identity to a corresponding polypeptide encoded by one of SEQ ID Nos. 1 or 4-6, respectively. In one embodiment, the influenza B virus of the invention is a recombinant influenza B virus having a particular amino acid residue at specified positions in one or more of PA, PB1, PB2, NP, M1 and/or NS1 and an amino acid sequence with at least 80%, e.g., 90%, 92%, 95%, 97%, 98%, or 99%, including any integer between 80 and 99, contiguous amino acid sequence identity to a corresponding polypeptide encoded by one of SEQ ID Nos. 1-6, such as a polypeptide with a residue that is a conservative substitution.

Also included are any combination of the selected amino acid residues at specified positions described herein.

Viral segments for PA, NP, M and/or NS that have the residues at the specified positions may be combined with viral segment for PB1, a viral segment for PB2, a viral segment for HA, and a viral segment for NA, to provide the reassortant vaccine viruses of the invention. In one embodiment, the HA viral segment in the reassortant virus is heterologous to the viral segments for PA, PB1, PB2, NP, M and NS. In one embodiment, the NA gene segment in the reassortant virus is heterologous to the viral segments for PA, PB1, PB2, NP, M and NS. In one embodiment, the HA viral segment in the reassortant virus has viral segments for PA, PB1, PB2, NP, M and NS from one influenza virus isolate or strain (“parent”), or a variant thereof, e.g., one with viral segments encoding influenza virus proteins with at least 95%, 96%, 97%, 98%, 99%, or 99.5% amino acid sequence identity, or having 1, 2, 5, 10, or 20 substitutions relative, to sequences in a parent influenza virus isolate or strain. In one embodiment, the parent strain has viral segments with sequences corresponding to at least one of SEQ ID Nos. 1-6, and the recombinant virus has at least one of the viral segments with at least one of the substitutions in PA, NS, NP or M described herein, and at least one of the parental viral segments. In one embodiment, the HA gene segment in the reassortant virus is a chimeric HA gene segment, e.g., a chimera of heterologous HA ectodomain sequences linked to HA signal peptide sequences and/or HA transmembrane domain sequences from the HA gene segment of the parent isolate or strain, or variant thereof. In one embodiment, the NA gene segment in the isolated recombinant virus is a chimeric NA gene segment e.g., a chimera of heterologous NA ectodomain sequences linked to NA transmembrane domain sequences from the NA gene segment of the parent isolate or strain, or variant thereof, and/or stalk sequences from the parent isolate or strain, or variant thereof, for instance, chimeras of influenza B virus NA and influenza A virus NA. In one embodiment, the NA gene segment in the isolated recombinant virus is a chimeric NA gene segment e.g., a chimera of heterologous NA ectodomain sequences linked to NA transmembrane domain sequences from the NA gene segment of the parent isolate or strain, or variant thereof, and/or stalk sequences from a second isolate or strain, or variant thereof. In one embodiment, the isolated recombinant virus has a heterologous HA gene segment, a heterologous NA gene segment, a chimeric HA gene segment, a chimeric NA gene segment, or any combination thereof. The nucleic acid sequences employed to prepare vRNA or cRNA may be ones that introduce the residues at the specified positions via recombinant methodology or may be selected as having the residues at the specified positions.

As described herein, an influenza virus isolate useful as a vaccine virus (e.g., reassortants of B/Yamagata/1/73 with viruses of the B/Yamagata- or B/Victoria-lineage) to carry heterologous gene segments for NA and/or HA, was serially passaged in MDCK cells, e.g., about 10-12-times although fewer passages may be employed, to obtain virus with enhanced replication in those cells. In one embodiment, viruses obtained after serial passage which have enhanced replication, have titers that are at least 0.5 to 1 or 2 logs higher than viruses that were not serially passaged. In one embodiment, viruses obtained after serial passage had substitutions in two or more internal gene segments relative to the parent virus.

Thus, for vaccine viruses that are to be grown or passaged in cells in culture, e.g., MDCK or Vero cells or eggs, selection of sequences with, or replacement of, the disclosed residues at the specified positions in one or more of PA, BM2, NP, M1 and/or NS1, that confer enhanced growth of the virus in cultured cells when employed with HA and NA sequences of interest, can result in significantly higher viral titers. Thus, the invention provides a method to select for influenza viruses with enhanced replication in cell culture. The method includes providing cells suitable for influenza vaccine production; serially culturing one or more influenza virus isolates in the cells; and isolating serially cultured virus with enhanced growth relative to the one or more isolates prior to serial culture. In one embodiment, the cells are canine or primate, e.g., human or monkey, cells.

The invention provides a plurality of influenza virus vectors of the invention, e.g., those useful to prepare reassortant viruses including 6:1:1 reassortants, 6:2 reassortants and 7:1 reassortants. A 6:1:1 reassortant within the scope of the present invention is an influenza virus with 6 internal gene segments from a vaccine virus, a NA gene segment from a different (second) viral isolate, and a HA gene segment from a third isolate; a 6:2 reassortant within the scope of the present invention is an influenza virus with 6 internal gene segments from a vaccine virus, and a NA gene segment and a HA gene segment from a different (second) viral isolate; and a 7:1 reassortant within the scope of the present invention is an influenza virus with 6 internal gene segments and a NA gene segment from a vaccine virus, and a HA gene segment from a different viral source than the vaccine virus, or an influenza virus with 6 internal gene segments and a HA gene segment from the vaccine virus, and a NA gene segment is from a different viral source than the vaccine virus.

In one embodiment of the invention, the plurality includes vectors for vRNA or cRNA production selected from a vector comprising a promoter operably linked to an influenza virus PA DNA, e.g., cDNA, linked to a transcription termination sequence, a vector comprising a promoter operably linked to an influenza virus PB1 DNA, e.g., cDNA, linked to a transcription termination sequence, a vector comprising a promoter operably linked to an influenza virus PB2 DNA, e.g., cDNA, linked to a transcription termination sequence, a vector comprising a promoter operably linked to an influenza virus HA DNA, e.g., cDNA, linked to a transcription termination sequence, a vector comprising a promoter operably linked to an influenza virus NP DNA, e.g., cDNA, linked to a transcription termination sequence, a vector comprising a promoter operably linked to an influenza virus NA DNA, e.g., cDNA, linked to a transcription termination sequence, a vector comprising a promoter operably linked to an influenza virus M DNA, e.g., cDNA, linked to a transcription termination sequence, and a vector comprising a operably linked to an influenza virus NS DNA linked to a transcription termination sequence. In one embodiment, the DNAs for vRNA or cRNA production of PB1, PB2, PA, NP, M, and NS, have sequences from an influenza virus that replicates to high titers in cultured mammalian cells such as MDCK cells, Vero cells or PER.C6® cells and also optionally embryonated eggs, and/or from a vaccine virus, e.g., one that does not cause significant disease in humans. The DNA, e.g., cDNA, for vRNA or cRNA production of NA may be from any NA, and the DNA for vRNA or cRNA production of HA may be from any HA. In one embodiment, the DNAs for vRNA or cRNA production may be for an influenza A or C virus. The DNAs for vRNA or cRNA production of NA and HA may be from different strains or isolates (6:1:1 reassortants) or from the same strain or isolate (6:2 reassortants), or the NA may be from the same strain or isolate as that for the internal genes (7:1 reassortant). The plurality also includes vectors for mRNA production selected from a vector encoding influenza virus PA, a vector encoding influenza virus PB1, a vector encoding influenza virus PB2, and a vector encoding influenza virus NP, and optionally one or more vectors encoding NP, NS, M, e.g., M1 and BM2, HA or NA. The vectors encoding viral proteins may further include a transcription termination sequence.

Viruses that may provide the internal genes for reassortants within the scope of the invention include viruses that have high titers in MDCK cells, e.g., titers of at least about 10⁵ PFU/mL, e.g., at least 10⁶ PFU/mL, 10⁷ PFU/mL or 10⁸ PFU/mL; high titers in embryonated eggs, e.g., titers of at least about 10⁷ EID₅₀/mL, e.g., at least 10⁸ EID₅₀/mL, 10⁹ EID₅₀/mL or 10¹⁰ EID₅₀/mL; high titers in cells such as MDCK cells, e.g., titers of at least about 10⁷ PFU/mL, e.g., at least 10⁸ PFU/mL, or high titers in two of more of those host cells.

In one embodiment, the titers of the reassortant viruses of the invention in cells such as MDCK cells or Vero cells may be over 1 log, 2 logs, 3 logs, or greater, than titers of the corresponding virus without particular residues at the specified positions.

In one embodiment, the DNAs for the internal genes for PB1, PB2, PA, NP, M, and NS encode proteins with substantially the same activity as a corresponding polypeptide encoded by one of SEQ ID NOs:1-6. As used herein, “substantially the same activity” includes an activity that is about 0.1%, 1%, 10%, 30%, 50%, 90%, e.g., up to 100% or more, or detectable protein level that is about 80%, 90% or more, the activity or protein level, respectively, of the corresponding full-length polypeptide. In one embodiment, the nucleic acid a sequence encoding a polypeptide which is substantially the same as, e.g., having at least 80%, e.g., 90%, 92%, 95%, 97%, 98%, or 99%, including any integer between 80 and 99, contiguous amino acid sequence identity to, a polypeptide encoded by one of SEQ ID NOs:1-6. In one embodiment, the isolated and/or purified nucleic acid molecule comprises a nucleotide sequence which is substantially the same as, e.g., having at least 50%, e.g., 60%, 70%, 80% or 90%, including any integer between 50 and 100, or more contiguous nucleic acid sequence identity to one of SEQ ID NOs:1-6 and, in one embodiment, also encodes a polypeptide having at least 80%, e.g., 90%, 92%, 95%, 97%, 98%, or 99%, including any integer between 80 and 99, contiguous amino acid sequence identity to a polypeptide encoded by one of SEQ ID NOs:1-6. In one embodiment, the influenza virus polypeptide has one or more, for instance, 2, 5, 10, 15, 20 or more, conservative amino acids substitutions, e.g., conservative substitutions of up to 10% or 20% of 2, 5, 10, 15, 20 or more, of a combination of conservative and non-conservative amino acids substitutions, e.g., conservative substitutions of up to 10% or 20% of the residues, or relative to a polypeptide encoded by one of SEQ ID NOs:1-6, and has a characteristic residue in one or more of PA, PB2, BM2, NP, M1, and/or NS1, relative to a polypeptide encoded by one of SEQ ID NOs:1-6. In one embodiment, the influenza virus polypeptide has one or more, for instance, 2, 3, 4, 5, 6, 7 or 8 conservative and/or nonconservative amino acid substitutions, relative to a polypeptide encoded by one of SEQ ID NOs:1-6.

The invention thus includes the use of isolated and purified vectors or plasmids, which express or encode influenza virus proteins, or express or encode influenza vRNA or cRNA, both native and recombinant vRNA or cRNA. The vectors may comprise influenza cDNA, e.g., influenza A, B or C DNA (see Fields Virology (Fields et al. (eds.), Lippincott, Williams and Wickens (2006), which is specifically incorporated by reference herein). Any suitable promoter or transcription termination sequence may be employed to express a protein or peptide, e.g., a viral protein or peptide, a protein or peptide of a nonviral pathogen, or a therapeutic protein or peptide.

A composition or plurality of vectors of the invention may also comprise a heterologous gene or open reading frame of interest, e.g., a foreign gene encoding an immunogenic peptide or protein useful as a vaccine or in gene replacement, for instance, may encode an epitope useful in a cancer therapy or vaccine, or a peptide or polypeptide useful in gene therapy. When preparing virus, the vector or plasmid comprising the gene or cDNA of interest may substitute for a vector or plasmid for an influenza viral gene or may be in addition to vectors or plasmids for all influenza viral genes. Thus, another embodiment of the invention comprises a composition or plurality of vectors as described above in which one of the vectors is replaced with, or further comprises, 5′ influenza virus sequences optionally including 5′ influenza virus coding sequences or a portion thereof, linked to a desired nucleic acid sequence, e.g., a desired cDNA, linked to 3′ influenza virus sequences optionally including 3′ influenza virus coding sequences or a portion thereof. In one embodiment, the desired nucleic acid sequence such as a cDNA is in an antisense (antigenomic) orientation. The introduction of such a vector in conjunction with the other vectors described above to a host cell permissive for influenza virus replication results in recombinant virus comprising vRNA or cRNA corresponding to the heterologous sequences of the vector.

The promoter in a vector for vRNA or cRNA production may be a RNA polymerase I promoter, a RNA polymerase II promoter, a RNA polymerase Ill promoter, a T7 promoter, or a T3 promoter, and optionally the vector comprises a transcription termination sequence such as a RNA polymerase I transcription termination sequence, a RNA polymerase II transcription termination sequence, a RNA polymerase Ill transcription termination sequence, or a ribozyme. Ribozymes within the scope of the invention include, but are not limited to, tetrahymena ribozymes, RNase P, hammerhead ribozymes, hairpin ribozymes, hepatitis ribozyme, as well as synthetic ribozymes. In one embodiment, the RNA polymerase I promoter is a human RNA polymerase I promoter.

The promoter or transcription termination sequence in a vRNA, cRNA or virus protein expression vector may be the same or different relative to the promoter or any other vector. In one embodiment, the vector or plasmid which expresses influenza vRNA or cRNA comprises a promoter suitable for expression in at least one particular host cell, e.g., avian or mammalian host cells such as canine, feline, equine, bovine, ovine, or primate cells including human cells, or for expression in more than one host.

In one embodiment, at least one vector for vRNA or cRNA comprises a RNA polymerase II promoter linked to a ribozyme sequence linked to viral coding sequences linked to another ribozyme sequences, optionally linked to a RNA polymerase II transcription termination sequence. In one embodiment, at least 2, e.g., 3, 4, 5, 6, 7 or 8, vectors for vRNA or cRNA production comprise a RNA polymerase II promoter, a first ribozyme sequence, which is 5′ to a sequence corresponding to viral sequences including viral coding sequences, which is 5′ to a second ribozyme sequence, which is 5′ to a transcription termination sequence. Each RNA polymerase II promoter in each vRNA or cRNA vector may be the same or different as the RNA polymerase II promoter in any other vRNA or cRNA vector. Similarly, each ribozyme sequence in each vRNA or cRNA vector may be the same or different as the ribozyme sequences in any other vRNA or cRNA vector. In one embodiment, the ribozyme sequences in a single vector are not the same.

In one embodiment, the invention provides a plurality of influenza virus vectors for a reassortant, comprising a vector for vRNA or cRNA production comprising a promoter operably linked to an influenza virus PA DNA, e.g., cDNA, linked to a transcription termination sequence, a vector for vRNA or cRNA production comprising a promoter operably linked to an influenza virus PB1 DNA, e.g., cDNA, linked to a transcription termination sequence, a vector for vRNA or cRNA production comprising a promoter operably linked to an influenza virus PB2 DNA, e.g., cDNA, linked to a transcription termination sequence, a vector for vRNA or cRNA production comprising a promoter operably linked to an influenza virus HA DNA, e.g., cDNA, linked to a transcription termination sequence, a vector for vRNA or cRNA production comprising a promoter operably linked to an influenza virus NP DNA, e.g., cDNA, linked to a transcription termination sequence, a vector for vRNA or cRNA production comprising a promoter operably linked to an influenza virus NA DNA, e.g., cDNA, linked to a transcription termination sequence, a vector for vRNA or cRNA production comprising a promoter operably linked to an influenza virus M DNA, e.g., cDNA, linked to a transcription termination sequence, and a vector for vRNA or cRNA production comprising a promoter operably linked to an influenza virus NS DNA, e.g., cDNA, linked to a transcription termination sequence, wherein the DNAs for PB1, PB2, PA, NP, NS, and M are from one or more influenza vaccine seed viruses and contain two or more of the characteristic residues at the specified position(s); and a vector for mRNA production comprising a promoter operably linked to a DNA segment encoding influenza virus PA, a vector for mRNA production comprising a promoter operably linked to a DNA segment encoding influenza virus PB1, a vector for mRNA production comprising a promoter operably linked to a DNA segment encoding influenza virus PB2, and a vector for mRNA production comprising a promoter operably linked to a DNA segment encoding influenza virus NP, and optionally a vector for mRNA production comprising a promoter operably linked to a DNA segment encoding influenza virus HA, a vector for mRNA production comprising a promoter operably linked to a DNA segment encoding influenza virus NA, a vector for mRNA production comprising a promoter operably linked to a DNA segment encoding influenza virus M1, a vector for mRNA production comprising a promoter operably linked to a DNA segment encoding influenza virus BM2, or a vector for mRNA production comprising a promoter operably linked to a DNA segment encoding influenza virus NS2. In one embodiment, at least one vector comprises sequences corresponding to those encoding PB1, PB2, PA, NP, M, or NS, or a portion thereof, having substantially the same activity as a corresponding polypeptide encoded by one of SEQ ID NOs:1-6, e.g., a sequence encoding a polypeptide with at least 80%, e.g., 85%, 90%, 92%, 95%, 98%, 99% or 100%, including any integer between 80 and 100, amino acid identity to a polypeptide encoded by one of SEQ ID NOs:1-6. Optionally, two vectors may be employed in place of the vector comprising a promoter operably linked to an influenza virus M cDNA linked to a transcription termination sequence, e.g., a vector comprising a promoter operably linked to an influenza virus M1 cDNA linked to a transcription termination sequence and a vector comprising a promoter operably linked to an influenza virus BM2 cDNA linked to a transcription termination sequence.

A plurality of the vectors of the invention may be physically linked or each vector may be present on an individual plasmid or other, e.g., linear, nucleic acid delivery vehicle. In one embodiment, each vRNA or cRNA production vector is on a separate plasmid. In one embodiment, each mRNA production vector is on a separate plasmid.

The invention also provides a method to prepare influenza virus. The method comprises contacting a cell with a plurality of the vectors of the invention, e.g., sequentially or simultaneously, in an amount effective to yield infectious influenza virus. The invention also includes isolating virus from a cell contacted with the plurality of vectors. Thus, the invention further provides isolated virus, as well as a host cell contacted with the plurality of vectors or virus of the invention. In another embodiment, the invention includes contacting the cell with one or more vectors, either vRNA or cRNA or protein production vectors, prior to other vectors, either vRNA or protein production vectors. In one embodiment, the promoter for vRNA or cRNA vectors employed in the method is a RNA polymerase I promoter, a RNA polymerase II promoter, a RNA polymerase Ill promoter, a T3 promoter or a T7 promoter. In one embodiment, the RNA polymerase I promoter is a human RNA polymerase I promoter. In one embodiment, each vRNA or cRNA vector employed in the method is on a separate plasmid. In one embodiment, the vRNA or cRNA vectors employed in the method are on one plasmid or on two or three different plasmids. In one embodiment, each mRNA vector employed in the method is on a separate plasmid. In one embodiment, the mRNA vectors for PA, PB1, PB2 and NP employed in the method are on one plasmid or on two or three different plasmids.

In one embodiment, the invention provides a method to select for influenza viruses with enhanced replication in cell culture. The method includes providing cells suitable for influenza vaccine production; serially culturing one or more influenza virus isolates in the cells; and isolating serially cultured virus with enhanced growth relative to the one or more isolates prior to serial culture. In one embodiment, the cells are rodent or primate cells.

The methods of producing virus described herein, which do not require helper virus infection, are useful in viral mutagenesis studies, and in the production of vaccines (e.g., for AIDS, influenza, hepatitis B, hepatitis C, rhinovirus, filoviruses, malaria, herpes, and foot and mouth disease) and gene therapy vectors (e.g., for cancer, AIDS, adenosine deaminase, muscular dystrophy, ornithine transcarbamylase deficiency and central nervous system tumors). Thus, a virus for use in medical therapy (e.g., for a vaccine or gene therapy) is provided.

The invention also provides isolated viral polypeptides, and methods of preparing and using recombinant virus of the invention. The methods include administering to a host organism, e.g., a mammal, an effective amount of the influenza virus of the invention, e.g., an inactivated virus preparation, optionally in combination with an adjuvant and/or a carrier, e.g., in an amount effective to prevent or ameliorate infection of an animal such as a mammal by that virus or an antigenically closely related virus. In one embodiment, the virus is administered intramuscularly while in another embodiment, the virus is administered intranasally. In some dosing protocols, all doses may be administered intramuscularly or intranasally, while in others a combination of intramuscular and intranasal administration is employed. The vaccine may further contain other isolates of influenza virus including recombinant influenza virus, other pathogen(s), additional biological agents or microbial components, e.g., to form a multivalent vaccine. In one embodiment, intranasal vaccination, for instance containing with inactivated influenza virus, and a mucosal adjuvant may induce virus-specific IgA and neutralizing antibody in the nasopharynx as well as serum IgG.

The influenza virus of the invention may employed with other anti-virals, e.g., amantadine, rimantadine, and/or neuraminidase inhibitors, e.g., the virus may be administered separately, for instance, administered before and/or after, or in conjunction with, those anti-virals.

### BRIEF DESCRIPTION OF THE FIGURES

FIG. 1A-G. Nucleotide sequences for B/Yamagata/1/1973 (SEQ ID NOs: 1-6, 9, and 10) and amino acid sequences for HA (SEQ ID NO: 7) and NA (SEQ ID NO: 8).

FIGS. 2A-B. Overview of library passages in MDCK cells and the identification of high-yield (HY) candidates (HY(Yam) and HY(Vic)). Yamagata- and Victoria-lineage virus libraries with random mutations in the indicated vRNAs were passaged twelve times in MDCK cells, or passaged twice, mixed, and passaged ten more times. More than 700 virus plaques were picked for each virus lineage. Based on hemagglutination and virus titers, the top eight candidates for each lineage were identified in a step-wise selection process. Testing of combinatorial mutations led to the selection of RG(Yam) #8 and RG(Vic) #2. The introduction of additional mutations (identified in virus library screens in Vero cells) resulted in the high-yield vaccine backbones HY(Yam) and HY(Vic). HA titer, hemagglutination titer.

FIGS. 3A-B. Viral titers (growth kinetics) and hemagglutination (HA) titers for select high-yield candidates for the B/Yamagata-(A) and B/Victoria-(B) lineages. Viruses possessing high-yield vaccine backbones of the Yamagata-(A) and Victoria-lineages (B). Mutations detected in the candidate viruses are shown in Tables 1 and 2, respectively. Data were obtained from three independent experiments; shown are average titers±s.d. Statistical significance was determined by using the linear mixed model (*p<0.05; **p<0.01); P values are not shown if the titer of the high-yield vaccine candidate was lower than that of wild-type virus. The color of the asterisks indicates the comparison of the respective virus with WT virus.

FIGS. 4A-B. Viral titers and HA titers for regenerated high-yield candidates for the B/Yamagata-(A) and B/Victoria-(B) lineages. Mutations introduced into the Yamagata-(A) and Victoria-lineage (B) viruses are shown in Tables 3 and 4, respectively. Data were obtained from three independent experiments; shown are average titers±s.d. P values were calculated by using the linear mixed model (*p<0.05; **p<0.01); P values are not shown if the titer of the high-yield vaccine candidate was lower than that of wild-type virus. The color of the asterisks indicates the comparison of the respective virus with WT virus.

FIG. 5. Chimeric HA and NA constructs. The ectodomains of the influenza B virus HA (green) and NA (blue) proteins were inserted between the remaining sequences of the influenza A virus PR8 HA (purple) and NA (dark orange) vRNAs. Wide bars indicate coding regions; small bars indicate non-coding regions (NCRs). SP, signal peptide; TM, transmembrane domain; CT, cytoplasmic tail.

FIG. 6A-B. Growth kinetics and HA titers of high-yield Yamagata- and Victoria-lineage viruses. (A) The indicated Yamagata-lineage wild-type virus was compared with the Yamagata-lineage high-yield candidate RG(Yam) #8 (Table 3) and with RG(Yam) #8 possessing the PA-a2272t mutation; the latter virus was selected as lead candidate HY(Yam). (B) The indicated Victoria-lineage wild-type virus was compared with the Victoria-lineage high-yield candidate RG(Vic) #2 (Table 4) and with RG(Vic) #2 possessing the PA-a2272t mutation; the latter virus was selected as lead candidate HY(Vic). In both sets of experiments, MDCK cells were infected in triplicate with the indicated viruses at an MOI of 0.001 and incubated at 35° C. At the indicated time points, virus and hemagglutination titers were determined by performing plaque or hemagglutination assays, respectively. The values presented are the average of three independent experiments±SD. P values were calculated by using the linear mixed model (*P<0.05; **P<0.01). Red and blue asterisks indicate the comparison of the respective virus with WT virus; beige asterisks indicate the comparison between the viruses depicted in red and blue.

FIGS. 7A-F. Comparison of wild-type and high-yield viruses possessing different HA and NA vRNAs. Viruses possessing the HA and NA vRNAs of the indicated viruses in combination with the internal vRNA segments of the respective natural wild-type isolate (WT), or of HY(Yam) (A-C) or HY(Vic) (D-F) (the viruses indicated by the black graphs possess the eight wild-type vRNA segments of a human influenza B virus isolate). The values presented are the average of three independent experiments±SD. P values were calculated by using the linear mixed model described in the Methods section (*P<0.05; **P<0.01). Red asterisks indicate the comparison of the respective virus with WT virus.

FIGS. 8A-D. Exchange of HY(Yam) and HY(Vic) backbones. (A and B) Comparison of the virus and hemagglutination titers of two wild-type Yamagata-lineage viruses with viruses possessing the same HA and NA vRNAs in combination with the internal genes of HY(Yam) or HY(Vic). (C and D) Comparison of the virus and hemagglutination titers of two wild-type Victoria-lineage viruses with viruses possessing the same HA and NA vRNAs in combination with the internal genes of HY(Yam) or HY(Vic). The values presented are the average of three independent experiments±SD. P values were calculated by using the linear mixed model (*P<0.05; **P<0.01). Red and blue asterisks indicate the comparison of the respective virus with WT virus; beige asterisks indicate the comparison between the viruses depicted in red and blue.

FIGS. 9A-F. Comparison of high-yield influenza A and B vaccine virus backbones. (A and B). The virus yield and hemagglutination titers of: (i) the indicated wild-type viruses; (ii) viruses possessing the indicated HA and NA vRNAs in combination with the internal genes of HY(Yam); and (iii) viruses possessing the indicated type A/B chimeric HA and NA vRNAs in combination with the internal genes of high-yield influenza A virus. (C and D), were compared. Similar experiments were carried out for viruses of the Victoria lineage. (E and F) Comparison of the indicated wild-type and hybrid viruses in embryonated chicken eggs. The values presented are the average of three independent experiments±SD. The statistical significance was determined by using the linear mixed model (A-D), or by two-way ANOVA, followed by Tukey's post hoc test (E and F) (*P<0.05; **P<0.01); P values are not shown if the titer of the high-yield vaccine candidate was lower than that of wild-type virus. Red and blue asterisks indicate the comparison of the respective virus with WT virus; beige asterisks indicate the comparison between the viruses depicted in red and blue.

FIGS. 10A-B. Evaluation of the total viral protein yield and HA content of HY(Yam) and HY(Vic) viruses. A) Comparison of viruses possessing the HA and NA vRNAs of the indicated Yamagata-lineage viruses in combination with the internal vRNAs of the same natural wild-type virus (WT) or of HY(Yam). B) Comparison of viruses possessing the HA and NA vRNAs of the indicated Victoria-lineage viruses in combination with the internal vRNAs of the same wild-type virus (WT) or of HY(Vic). The total viral protein yield of MDCK cell-grown, sucrose gradient-purified virus samples is shown (Left and Center). PNGaseF treatment deglycosylates HA1 and HA2; this treatment was carried out because glycosylated HA2 migrates at a similar molecular weight asM1. The HA contents (Right) were calculated based on the total viral protein amounts and the relative amounts of HA. The values presented are the average of three independent experiments±SD. The statistical significance was assessed by using one-way ANOVA followed by Dunnett's test, comparing the total viral protein yield and HA content of wild-type viruses with that of recombinant high-yield vaccine viruses (*P<0.05; **P<0.01).

FIGS. 11A-F. Virulence of HY(Yam) and HY(Vic) viruses in mice. A-C) Comparison of a wild-type Yamagata-lineage virus (B/Massachusetts/2/2012), a virus possessing the B/Massachusetts/2/2012 HA and NA vRNAs in combination with the remaining vRNAs of B/Yamagata/1/73 (used for virus library generation), and a virus possessing the B/Massachusetts/2/2012 HA and NA vRNAs in combination with the remaining vRNAs of HY(Yam). D-F) Comparison of a wild-type Victoria-lineage virus (B/Brisbane/60/2008), a virus possessing the B/Brisbane/60/2008 HA and NA vRNAs in combination with the remaining vRNAs of B/Yamagata/1/73 (used for virus library generation), and a virus possessing the B/Brisbane/60/2008 HA and NA vRNAs in combination with the remaining vRNAs of HY(Vic). BALB/c mice (five per group) were inoculated intranasally with 10⁶ pfu of the indicated viruses and monitored daily for body weight changes (A and D) and survival (B and E). To assess virus replication in mice, 106 pfu of the indicated viruses were used to infect 10 additional mice. On days 3 and 6 post-infection, five mice in each group were killed, and lung virus titers were determined by use of plaque assays in MDCK cells (C and F). Statistical significance was assessed by using one-way ANOVA followed by Dunnett's test (*P<0.05; **P<0.01).

FIGS. 12A-B. Growth kinetics and hemagglutination titers of single reassortant viruses. A) Comparison of the parental virus used for Yamagata-lineage virus library generation (B/Yamagata/1/73 with the HA and NA vRNAs of B/Yokohama/UT-K31/2012) with viruses that also possess an individual vRNA of HY(Yam). B) Comparison of the parental virus used for Victoria-lineage virus library generation (i.e., B/Yamagata/1/73 with the HA and NA vRNAs of B/Yokohama/UT-K1A/2011) with viruses that also possess an individual vRNA of HY(Vic). Data were obtained from three independent experiments; shown are average titers±SD. The values presented are the average of three independent experiments±SD. Statistical significance was determined by using the linear mixed model (*P<0.05; **P<0.01). The color of the asterisks indicates the comparison of the respective virus with the comparator virus (depicted in black).

FIGS. 13A-D. Luciferase activity in mini-replicon assay at 35° C. Effect of mutations in the NP protein or PA and NS vRNAs on viral polymerase activity. 293T (A) or MDCK (B) cells were transfected with protein expression plasmids for the polymerase proteins and wild-type or mutant NP, and with a plasmid transcribing a virus-like RNA that encodes luciferase. Luciferase activity was measured 48 hours later. In parallel, MDCK cells were transfected with the protein expression plasmids described above, and with wild-type or mutant virus-like RNA encoding luciferase and possessing the indicated mutations in the non-coding regions of the PA vRNA (C) or NS vRNA (D). Luciferase activity was measured 48 hours later. Data were obtained from three independent experiments; shown are average titers±s.d. Statistical significance was determined by using one-way ANOVA, followed by Dunnett's test (*p<0.05; **p<0.01).

FIGS. 14A-B. Contribution of M1 mutations in HY backbones to HA, NP and M1 VLP incorporation and composition. 293T cells were transfected with protein expression plasmids for HA, NA, NP, BM2, NS2, and wild-type or mutant M1. At 48 hours post-transfection, cell lysates and VLPs in cell culture supernatants were Western blotted with anti-HA, anti-NP, and anti-M1 monoclonal antibodies (A). The intensity of the bands for HA, NP, and M1 was quantified by using ImageJ software (NIH), and the relative percentages of HA, NP, and M1 in VLPs are shown in (B). Data were obtained from three independent experiments; shown are average titers±s.d. Statistical significance was determined by using one-way ANOVA, followed by Tukey's post hoc test (*p<0.05; **p<0.01).

FIGS. 15A-B. Effect of NS1 mutation on IFN activity. A) To compare the ability of wild-type and mutant NS1 to interfere with IFN-β synthesis, 293T cells were transfected with a wild-type or NS1 protein expression plasmid and with the reporter plasmid pGL-IFN-β, which encodes the firefly luciferase protein under the control of the IFN-β promoter. Cells were incubated for 24 hours, infected with Sendai virus at an MOI of 5, again incubated for 24 hours, and then lysed to measure firefly luciferase. B) To determine the ability of wild-type and mutant NS1 to interfere with the synthesis of IFN-β-stimulated genes, 293T cells were transfected with a wild-type or mutant NS1 protein expression plasmid and with the reporter plasmid pISRE-Luc (which encodes the firefly luciferase protein under the control of an interferon-regulated promoter). Twenty-four hours later, cells were stimulated with human IFN-β. Forty-eight hours after transfection, we measured luciferase activity. Data were obtained from three independent experiments; shown are average titers±s.d. Statistical significance was determined by using one-way ANOVA, followed by Tukey's post hoc test (*p<0.05; **p<0.01).

FIG. 16A-I. Nucleotide sequences for viral segments in select HY clones (PB2 and PB1 from Yamagata/1/73; SEQ ID Nos. 11-12); PA from Yamagata/1/73 having a1406g/c1445t/a2272t (SEQ ID NO:13); NP from Yamagata/1/73 having P40S, c500t (SEQ ID NO:14) or P40S/M204T, c500t (SEQ ID NO:15); M from Yamagata/1/73 having R77K (SEQ ID NO:16) or M86T (SEQ ID NO:17); and NS from Yamagata/1/73 having a39g K176Q (SEQ ID NO:18) or 38(+1)g. (SEQ ID NO:19).

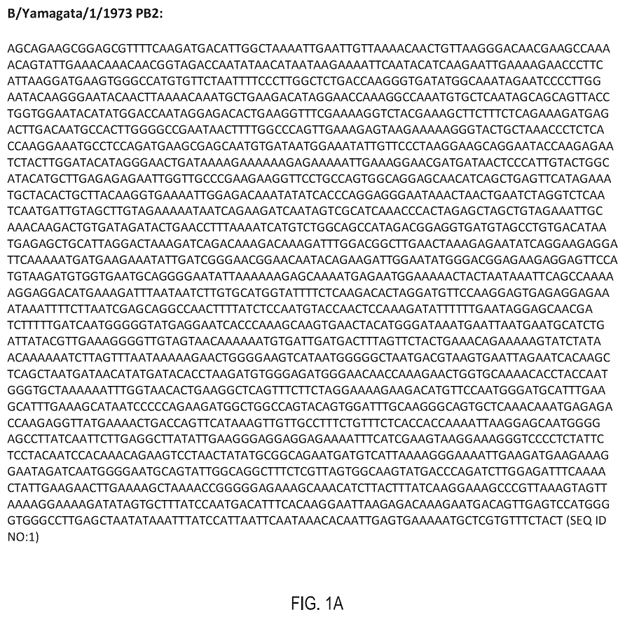

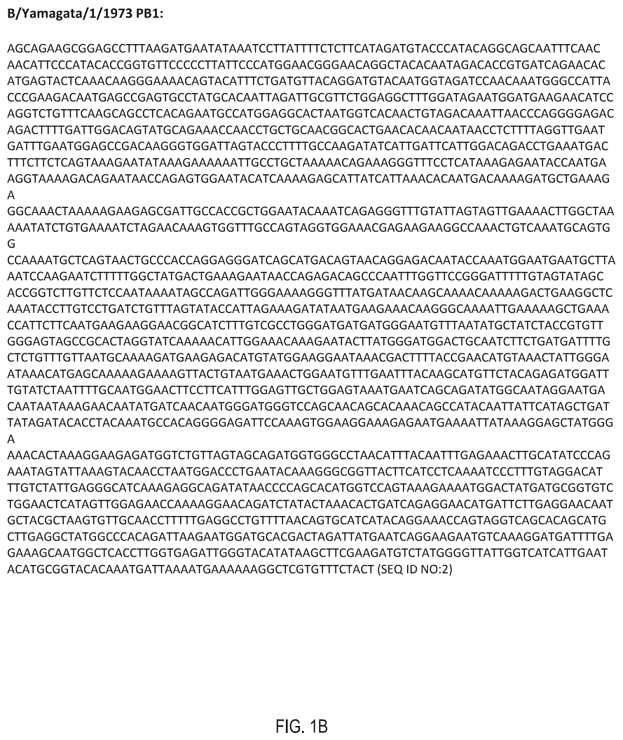

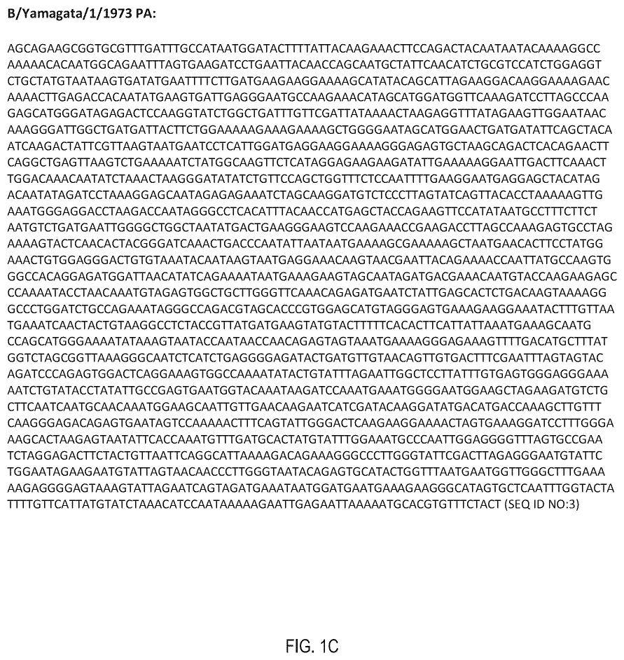

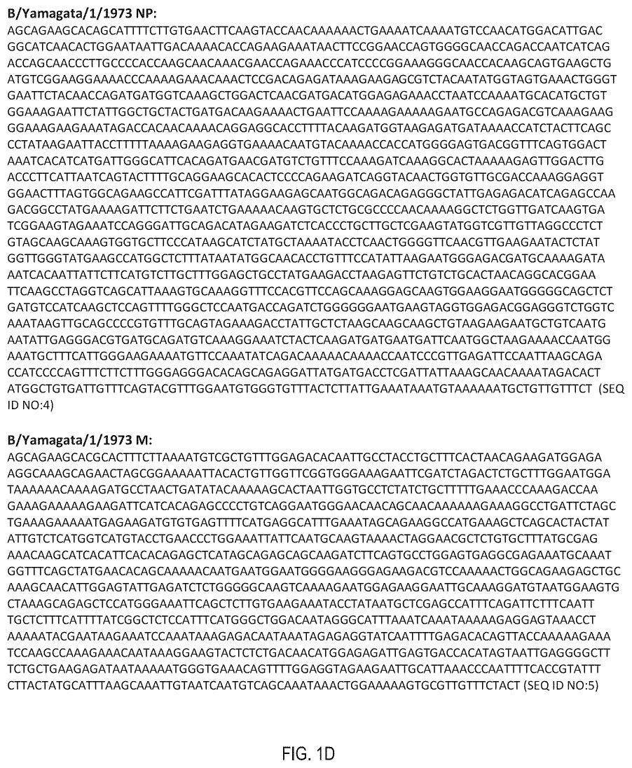

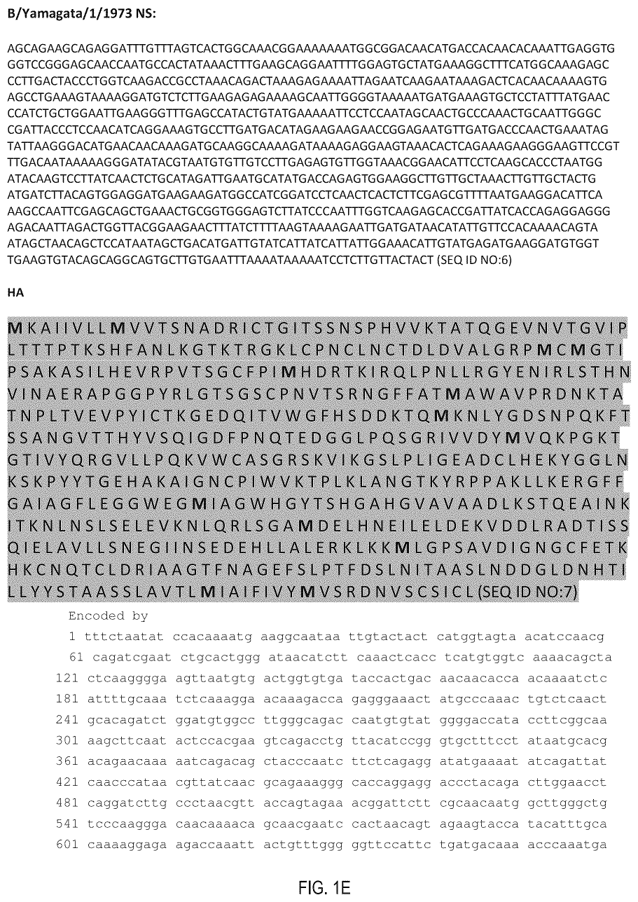

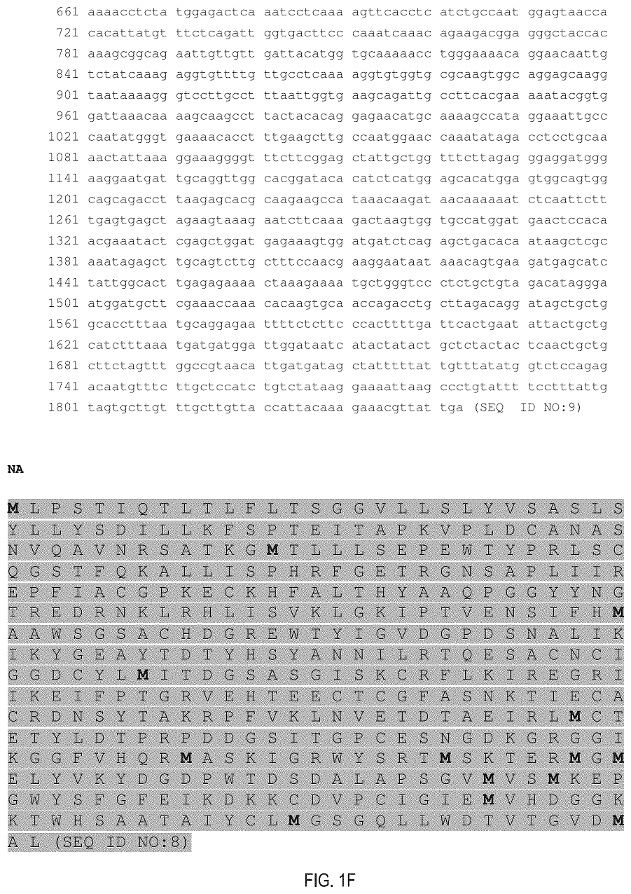

### DETAILED DESCRIPTION

### Definitions

As used herein, the term “isolated” refers to in vitro preparation and/or isolation of a nucleic acid molecule, e.g., vector or plasmid, peptide or polypeptide (protein), or virus of the invention, so that it is not associated with in vivo substances, or is substantially purified from in vitro substances. An isolated virus preparation is generally obtained by in vitro culture and propagation, and/or via passage in eggs, and is substantially free from other infectious agents.

As used herein, “substantially purified” means the object species is the predominant species, e.g., on a molar basis it is more abundant than any other individual species in a composition, and preferably is at least about 80% of the species present, and optionally 90% or greater, e.g., 95%, 98%, 99% or more, of the species present in the composition.

As used herein, “substantially free” means below the level of detection for a particular infectious agent using standard detection methods for that agent.

A “recombinant” virus is one which has been manipulated in vitro, e.g., using recombinant DNA techniques, to introduce changes to the viral genome. Reassortant viruses can be prepared by recombinant or nonrecombinant techniques.

As used herein, the term “recombinant nucleic acid” or “recombinant DNA sequence or segment” refers to a nucleic acid, e.g., to DNA, that has been derived or isolated from a source, that may be subsequently chemically altered in vitro, so that its sequence is not naturally occurring, or corresponds to naturally occurring sequences that are not positioned as they would be positioned in the native genome. An example of DNA “derived” from a source, would be a DNA sequence that is identified as a useful fragment, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, for use in the invention, by the methodology of genetic engineering.

As used herein, a “heterologous” influenza virus gene or gene segment is from an influenza virus source that is different than a majority of the other influenza viral genes or gene segments in a recombinant, e.g., reassortant, influenza virus.

The terms “isolated polypeptide” “isolated peptide” or “isolated protein” include a polypeptide, peptide or protein encoded by cDNA or recombinant RNA including one of synthetic origin, or some combination thereof.

The term “recombinant protein” or “recombinant polypeptide” as used herein refers to a protein molecule expressed from a recombinant DNA molecule. In contrast, the term “native protein” is used herein to indicate a protein isolated from a naturally occurring (i.e., a nonrecombinant) source. Molecular biological techniques may be used to produce a recombinant form of a protein with identical properties as compared to the native form of the protein.

Methods of alignment of sequences for comparison are well known in the art. Thus, the determination of percent identity between any two sequences can be accomplished using a mathematical algorithm.

Computer implementations of these mathematical algorithms can be utilized for comparison of sequences to determine sequence identity. Alignments using these programs can be performed using the default parameters. Software for performing BLAST analyses is publicly available through the National Center for Biotechnology Information (http://www.ncbi.nlm.nih.gov/). The algorithm may involve first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm may also perform a statistical analysis of the similarity between two sequences. One measure of similarity provided by the BLAST algorithm may be the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

The BLASTN program (for nucleotide sequences) may use as defaults a wordlength (W) of 11, an expectation (E) of 10, a cutoff of 100, M=5, N=−4, and a comparison of both strands. For amino acid sequences, the BLASTP program may use as defaults a wordlength (W) of 3, an expectation (E) of 10, and the BLOSUM62 scoring matrix. See http://www.ncbi.n1m.nih.gov. Alignment may also be performed manually by inspection.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

### Influenza B Virus Structure and Propagation

Influenza B viruses possess a genome of eight single-stranded negative-sense viral RNAs (vRNAs) that encode at least ten proteins. The influenza virus life cycle begins with binding of the hemagglutinin (HA) to sialic acid-containing receptors on the surface of the host cell, followed by receptor-mediated endocytosis. The low pH in late endosomes triggers a conformational shift in the HA, thereby exposing the N-terminus of the HA2 subunit (the so-called fusion peptide). The fusion peptide initiates the fusion of the viral and endosomal membrane, and the matrix protein (M1) and RNP complexes are released into the cytoplasm. RNPs consist of the nucleoprotein (NP), which encapsidates vRNA, and the viral polymerase complex, which is formed by the PA, PB1, and PB2 proteins. RNPs are transported into the nucleus, where transcription and replication take place. The RNA polymerase complex catalyzes three different reactions: synthesis of an mRNA with a 5 cap and 3′ polyA structure, of a full-length complementary RNA (cRNA), and of genomic vRNA using the cRNA as a template. Newly synthesized vRNAs, NP, and polymerase proteins are then assembled into RNPs, exported from the nucleus, and transported to the plasma membrane, where budding of progeny virus particles occurs. The neuraminidase (NA) protein plays a crucial role late in infection by removing sialic acid from sialyloligosaccharides, thus releasing newly assembled virions from the cell surface and preventing the self aggregation of virus particles. Although virus assembly involves protein-protein and protein-vRNA interactions, the nature of these interactions is largely unknown.

### Influenza B Viruses of the Invention

Mutations that increase the replicative ability of viruses in cell culture and/or embryonated chicken eggs are useful to amplify influenza viruses and to establish robust influenza vaccine platforms. Currently, most influenza B vaccines are generated in embryonated chicken eggs. Influenza vaccines generated in MDCK cells are now approved for human use in the U.S. and in Europe, and influenza vaccines derived from Vero cells are approved for human use in Europe. As described herein, virus libraries possessing random mutations in the ‘internal’ viral genes (viral genes except those encoding the viral surface glycoproteins HA and NA) of a vaccine virus isolate, e.g., internal genes of B/Yamagata/1/73 with NA and HA genes from B/Yokohama/UT-K31/2012 (representing Yamagata-lineage) or NA and HA genes from B/Yokohama/UT-K1A/2011 (representing Victoria-lineage), were generated and passaged in cells, e.g., MDCK or Vero cells. The identified mutations result in higher virus titers in cells (that may also increase virus titers in heterologous cells and/or embryonated chicken eggs), allowing more efficient influenza B virus growth and more cost-effective vaccine production. In addition to mutations in the coding regions of the internal viral segments and viral glycoproteins, mutations in non-coding regions were observed to increase viral titers, e.g., g1795a in the NP segment, a39g in the NS segment, an additional g after position 38 in the NS segment, or g2213a or a2272t in the PA segment. The resulting coding sequences conferring enhanced growth may be also codon-usage optimized, e.g., optimized for expression in mammalian cells such as canine cells or primate cells, or avian cells, e.g., chicken embryos. The mutations can be used in various combinations, with results influenced by the cell line (or egg) in use and the desired level of improvement in the replication of the virus. One or more selected mutations may be introduced into one or more internal viral genes of a vaccine virus isolate, or one or more internal viral genes having one or more of the mutations may be selected for inclusion in a reassortant useful as a vaccine virus. That virus may then be combined with other viruses, e.g., one or more influenza A viruses and/or one or more other influenza B viruses, to form a multivalent vaccine.

Cell Lines that can be Used in the Present Invention

Any cell, e.g., any avian or mammalian cell, such as a human, e.g., 293T or PER.C6® cells, or canine, e.g., MDCK, bovine, equine, feline, swine, ovine, rodent, for instance mink, e.g., MvLu1 cells, or hamster, e.g., CHO cells, or non-human primate, e.g., Vero cells, including mutant cells, which supports efficient replication of influenza virus can be employed to isolate and/or propagate influenza viruses. Isolated viruses can be used to prepare a reassortant virus. In one embodiment, host cells for vaccine production are continuous mammalian or avian cell lines or cell strains. A complete characterization of the cells to be used, may be conducted so that appropriate tests for purity of the final product can be included. Data that can be used for the characterization of a cell includes (a) information on its origin, derivation, and passage history; (b) information on its growth and morphological characteristics; (c) results of tests of adventitious agents; (d) distinguishing features, such as biochemical, immunological, and cytogenetic patterns which allow the cells to be clearly recognized among other cell lines; and (e) results of tests for tumorigenicity. In one embodiment, the passage level, or population doubling, of the host cell used is as low as possible.

In one embodiment, the cells are WHO certified, or certifiable, continuous cell lines. The requirements for certifying such cell lines include characterization with respect to at least one of genealogy, growth characteristics, immunological markers, virus susceptibility tumorigenicity and storage conditions, as well as by testing in animals, eggs, and cell culture. Such characterization is used to confirm that the cells are free from detectable adventitious agents. In some countries, karyology may also be required. In addition, tumorigenicity may be tested in cells that are at the same passage level as those used for vaccine production. The virus may be purified by a process that has been shown to give consistent results, before vaccine production (see, e.g., World Health Organization, 1982).

Virus produced by the host cell may be highly purified prior to vaccine or gene therapy formulation. Generally, the purification procedures result in extensive removal of cellular DNA and other cellular components, and adventitious agents. Procedures that extensively degrade or denature DNA may also be used.

### Influenza Vaccines

A vaccine of the invention includes an isolated recombinant influenza virus of the invention, and optionally one or more other isolated viruses including other isolated influenza viruses, one or more immunogenic proteins or glycoproteins of one or more isolated influenza viruses or one or more other pathogens, e.g., an immunogenic protein from one or more bacteria, non-influenza viruses, yeast or fungi, or isolated nucleic acid encoding one or more viral proteins (e.g., DNA vaccines) including one or more immunogenic proteins of the isolated influenza virus of the invention. In one embodiment, the influenza viruses of the invention may be vaccine vectors for influenza virus or other pathogens.

A complete virion vaccine may be concentrated by ultrafiltration and then purified by zonal centrifugation or by chromatography. Viruses other than the virus of the invention, such as those included in a multivalent vaccine, may be inactivated before or after purification using formalin or beta-propiolactone, for instance.

A subunit vaccine comprises purified glycoproteins. Such a vaccine may be prepared as follows: using viral suspensions fragmented by treatment with detergent, the surface antigens are purified, by ultracentrifugation for example. The subunit vaccines thus contain mainly HA protein, and also NA. The detergent used may be cationic detergent for example, such as hexadecyl trimethyl ammonium bromide (Bachmeyer, 1975), an anionic detergent such as ammonium deoxycholate (Laver & Webster, 1976); or a nonionic detergent such as that commercialized under the name TRITON X100. The hemagglutinin may also be isolated after treatment of the virions with a protease such as bromelin, and then purified. The subunit vaccine may be combined with an attenuated virus of the invention in a multivalent vaccine.

A split vaccine comprises virions which have been subjected to treatment with agents that dissolve lipids. A split vaccine can be prepared as follows: an aqueous suspension of the purified virus obtained as above, inactivated or not, is treated, under stirring, by lipid solvents such as ethyl ether or chloroform, associated with detergents. The dissolution of the viral envelope lipids results in fragmentation of the viral particles. The aqueous phase is recuperated containing the split vaccine, constituted mainly of hemagglutinin and neuraminidase with their original lipid environment removed, and the core or its degradation products. Then the residual infectious particles are inactivated if this has not already been done. The split vaccine may be combined with an attenuated virus of the invention in a multivalent vaccine.

Inactivated Vaccines. Inactivated influenza virus vaccines are provided by inactivating replicated virus using known methods, such as, but not limited to, formalin or β-propiolactone treatment. Inactivated vaccine types that can be used in the invention can include whole-virus (WV) vaccines or subvirion (SV) (split) vaccines. The WV vaccine contains intact, inactivated virus, while the SV vaccine contains purified virus disrupted with detergents that solubilize the lipid-containing viral envelope, followed by chemical inactivation of residual virus.

In addition, vaccines that can be used include those containing the isolated HA and NA surface proteins, which are referred to as surface antigen or subunit vaccines.

Live Attenuated Virus Vaccines. Live, attenuated influenza virus vaccines, such as those including a recombinant virus of the invention can be used for preventing or treating influenza virus infection. Attenuation may be achieved in a single step by transfer of attenuated genes from an attenuated donor virus to a replicated isolate or reassorted virus according to known methods. Since resistance to influenza A virus is mediated primarily by the development of an immune response to the HA and/or NA glycoproteins, the genes coding for these surface antigens come from the reassorted viruses or clinical isolates. The attenuated genes are derived from an attenuated parent. In this approach, genes that confer attenuation generally do not code for the HA and NA glycoproteins.

Viruses (donor influenza viruses) are available that are capable of reproducibly attenuating influenza viruses, e.g., a cold adapted (ca) donor virus can be used for attenuated vaccine production. Live, attenuated reassortant virus vaccines can be generated by mating the ca donor virus with a virulent replicated virus. Reassortant progeny are then selected at 25° C. (restrictive for replication of virulent virus), in the presence of an appropriate antiserum, which inhibits replication of the viruses bearing the surface antigens of the attenuated ca donor virus. Useful reassortants are: (a) infectious, (b) attenuated for seronegative non-adult mammals and immunologically primed adult mammals, (c) immunogenic and (d) genetically stable. The immunogenicity of the ca reassortants parallels their level of replication. Thus, the acquisition of the six transferable genes of the ca donor virus by new wild-type viruses has reproducibly attenuated these viruses for use in vaccinating susceptible mammals both adults and non-adult.

Other attenuating mutations can be introduced into influenza virus genes by site-directed mutagenesis to rescue infectious viruses bearing these mutant genes. Attenuating mutations can be introduced into non-coding regions of the genome, as well as into coding regions. Such attenuating mutations can also be introduced into genes other than the HA or NA, e.g., the PB2 polymerase gene. Thus, new donor viruses can also be generated bearing attenuating mutations introduced by site-directed mutagenesis, and such new donor viruses can be used in the production of live attenuated reassortants vaccine candidates in a manner analogous to that described above for the ca donor virus. Similarly, other known and suitable attenuated donor strains can be reassorted with influenza virus to obtain attenuated vaccines suitable for use in the vaccination of mammals.

In one embodiment, such attenuated viruses maintain the genes from the virus that encode antigenic determinants substantially similar to those of the original clinical isolates. This is because the purpose of the attenuated vaccine is to provide substantially the same antigenicity as the original clinical isolate of the virus, while at the same time lacking pathogenicity to the degree that the vaccine causes minimal chance of inducing a serious disease condition in the vaccinated mammal.

The viruses in a multivalent vaccine can thus be attenuated or inactivated, formulated and administered, according to known methods, as a vaccine to induce an immune response in an animal, e.g., a mammal. Methods are well-known in the art for determining whether such attenuated or inactivated vaccines have maintained similar antigenicity to that of the clinical isolate or high growth strain derived therefrom. Such known methods include the use of antisera or antibodies to eliminate viruses expressing antigenic determinants of the donor virus: chemical selection (e.g., amantadine or rimantidine); HA and NA activity and inhibition; and nucleic acid screening (such as probe hybridization or PCR) to confirm that donor genes encoding the antigenic determinants (e.g., HA or NA genes) are not present in the attenuated viruses.

### Pharmaceutical Compositions

Pharmaceutical compositions of the present invention, suitable for inoculation, e.g., nasal, parenteral or oral administration, comprise one or more influenza virus isolates, e.g., one or more attenuated or inactivated influenza viruses, a subunit thereof, isolated protein(s) thereof, and/or isolated nucleic acid encoding one or more proteins thereof, optionally further comprising sterile aqueous or non-aqueous solutions, suspensions, and emulsions. The compositions can further comprise auxiliary agents or excipients, as known in the art. The composition of the invention is generally presented in the form of individual doses (unit doses).

Conventional vaccines generally contain about 0.1 to 200 μg, e.g., 30 to 100 μg, of HA from each of the strains entering into their composition. The vaccine forming the main constituent of the vaccine composition of the invention may comprise a single influenza virus, or a combination of influenza viruses, for example, at least two or three influenza viruses, including one or more reassortant(s).

Preparations for parenteral administration include sterile aqueous or non-aqueous solutions, suspensions, and/or emulsions, which may contain auxiliary agents or excipients known in the art. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption. Liquid dosage forms for oral administration may generally comprise a liposome solution containing the liquid dosage form. Suitable forms for suspending liposomes include emulsions, suspensions, solutions, syrups, and elixirs containing inert diluents commonly used in the art, such as purified water. Besides the inert diluents, such compositions can also include adjuvants, wetting agents, emulsifying and suspending agents, or sweetening, flavoring, or perfuming agents.

When a composition of the present invention is used for administration to an individual, it can further comprise salts, buffers, adjuvants, or other substances which are desirable for improving the efficacy of the composition. For vaccines, adjuvants, substances which can augment a specific immune response, can be used. Normally, the adjuvant and the composition are mixed prior to presentation to the immune system, or presented separately, but into the same site of the organism being immunized.

Heterogeneity in a vaccine may be provided by mixing replicated influenza viruses for at least two influenza virus strains, such as 2-20 strains or any range or value therein. Vaccines can be provided for variations in a single strain of an influenza virus, using techniques known in the art.

A pharmaceutical composition according to the present invention may further or additionally comprise at least one chemotherapeutic compound, for example, for gene therapy, immunosuppressants, anti-inflammatory agents or immune enhancers, and for vaccines, chemotherapeutics including, but not limited to, gamma globulin, amantadine, guanidine, hydroxybenzimidazole, interferon-α, interferon-β, interferon-γ, tumor necrosis factor-alpha, thiosemicarbarzones, methisazone, rifampin, ribavirin, a pyrimidine analog, a purine analog, foscarnet, phosphonoacetic acid, acyclovir, dideoxynucleosides, a protease inhibitor, or ganciclovir.

The composition can also contain variable but small quantities of endotoxin-free formaldehyde, and preservatives, which have been found safe and not contributing to undesirable effects in the organism to which the composition is administered.

### Pharmaceutical Purposes

The administration of the composition (or the antisera that it elicits) may be for either a “prophylactic” or “therapeutic” purpose. When provided prophylactically, the compositions of the invention which are vaccines are provided before any symptom or clinical sign of a pathogen infection becomes manifest. The prophylactic administration of the composition serves to prevent or attenuate any subsequent infection. When provided prophylactically, the gene therapy compositions of the invention, are provided before any symptom or clinical sign of a disease becomes manifest. The prophylactic administration of the composition serves to prevent or attenuate one or more symptoms or clinical signs associated with the disease.

When provided therapeutically, a viral vaccine is provided upon the detection of a symptom or clinical sign of actual infection. The therapeutic administration of the compound(s) serves to attenuate any actual infection. When provided therapeutically, a gene therapy composition is provided upon the detection of a symptom or clinical sign of the disease. The therapeutic administration of the compound(s) serves to attenuate a symptom or clinical sign of that disease.

Thus, a vaccine composition of the present invention may be provided either before the onset of infection (so as to prevent or attenuate an anticipated infection) or after the initiation of an actual infection. Similarly, for gene therapy, the composition may be provided before any symptom or clinical sign of a disorder or disease is manifested or after one or more symptoms are detected.

A composition is said to be “pharmacologically acceptable” if its administration can be tolerated by a recipient mammal. Such an agent is said to be administered in a “therapeutically effective amount” if the amount administered is physiologically significant. A composition of the present invention is physiologically significant if its presence results in a detectable change in the physiology of a recipient patient, e.g., enhances at least one primary or secondary humoral or cellular immune response against at least one strain of an infectious influenza virus.

The “protection” provided need not be absolute, i.e., the influenza infection need not be totally prevented or eradicated, if there is a statistically significant improvement compared with a control population or set of mammals. Protection may be limited to mitigating the severity or rapidity of onset of symptoms or clinical signs of the influenza virus infection.

### Pharmaceutical Administration

A composition of the present invention may confer resistance to one or more pathogens, e.g., one or more influenza virus strains, by either passive immunization or active immunization. In active immunization, an attenuated live vaccine composition is administered prophylactically to a host (e.g., a mammal), and the host's immune response to the administration protects against infection and/or disease. For passive immunization, the elicited antisera can be recovered and administered to a recipient suspected of having an infection caused by at least one influenza virus strain. A gene therapy composition of the present invention may yield prophylactic or therapeutic levels of the desired gene product by active immunization.

In one embodiment, the vaccine is provided to a mammalian female (at or prior to pregnancy or parturition), under conditions of time and amount sufficient to cause the production of an immune response which serves to protect both the female and the fetus or newborn (via passive incorporation of the antibodies across the placenta or in the mother's milk).

The present invention thus includes methods for preventing or attenuating a disorder or disease, e.g., an infection by at least one strain of pathogen. As used herein, a vaccine is said to prevent or attenuate a disease if its administration results either in the total or partial attenuation (i.e., suppression) of a clinical sign or condition of the disease, or in the total or partial immunity of the individual to the disease. As used herein, a gene therapy composition is said to prevent or attenuate a disease if its administration results either in the total or partial attenuation (i.e., suppression) of a clinical sign or condition of the disease, or in the total or partial immunity of the individual to the disease.

A composition having at least one influenza virus of the present invention, including one which is attenuated and one or more other isolated viruses, one or more isolated viral proteins thereof, one or more isolated nucleic acid molecules encoding one or more viral proteins thereof, or a combination thereof, may be administered by any means that achieve the intended purposes.

For example, administration of such a composition may be by various parenteral routes such as subcutaneous, intravenous, intradermal, intramuscular, intraperitoneal, intranasal, oral or transdermal routes. Parenteral administration can be accomplished by bolus injection or by gradual perfusion over time.

A typical regimen for preventing, suppressing, or treating an influenza virus related pathology, comprises administration of an effective amount of a vaccine composition as described herein, administered as a single treatment, or repeated as enhancing or booster dosages, over a period up to and including between one week and about 24 months, or any range or value therein.

According to the present invention, an “effective amount” of a composition is one that is sufficient to achieve a desired effect. It is understood that the effective dosage may be dependent upon the species, age, sex, health, and weight of the recipient, kind of concurrent treatment, if any, frequency of treatment, and the nature of the effect wanted. The ranges of effective doses provided below are not intended to limit the invention and represent dose ranges.

The dosage of a live, attenuated or killed virus vaccine for an animal such as a mammalian adult organism may be from about 10²-10¹⁵, e.g., 10³-10¹², plaque forming units (PFU), or any range or value therein. The dose of inactivated vaccine may range from about 0.1 to 1000, e.g., 30 to 100 μg, of HA protein. However, the dosage should be a safe and effective amount as determined by conventional methods, using existing vaccines as a starting point.

The dosage of immunoreactive HA in each dose of replicated virus vaccine may be standardized to contain a suitable amount, e.g., 30 to 100 μg or any range or value therein, or the amount recommended by government agencies or recognized professional organizations. The quantity of NA can also be standardized, however, this glycoprotein may be labile during purification and storage.

The dosage of immunoreactive HA in each dose of replicated virus vaccine can be standardized to contain a suitable amount, e.g., 1-50 μg or any range or value therein, or the amount recommended by the U.S. Public Health Service (PHS), which is usually 15 μg per component for older children (greater than or equal to 3 years of age), and 7.5 μg per component for children less than 3 years of age. The quantity of NA can also be standardized, however, this glycoprotein can be labile during the processor purification and storage (Kendal et al., 1980; Kerr et al., 1975). Each 0.5-ml dose of vaccine may contains approximately 1-50 billion virus particles, and preferably 10 billion particles.

### EXEMPLARY EMBODIMENTS

In one embodiment, the recombinant or reassortant influenza B virus has an amino acid that results in enhanced replication in MDCK cells, e.g., residues in HA including but not limited to a residue other than T at position 34, other than K at position 129, other than N at position 168, other than T at position 196, or any combination thereof; residues in NA including but not limited to a residue other than residue N at position 169, and/or other than G at position 434; residues in NP including but are not limited to a residue other than alanine (A) at position 28, other than P at position 40, other than P a position 51, other than E at position 52, other than S at position 57, other than M at position 204, other than g at nucleotide position 1795, or any combination thereof; residues in M1 including but not limited to a residue other than G at position 34, other than aspartic acid (D) at position 54, other than R at position 77, other than M at position 86; residues in BM2 including but not limited to a residue other than H at position 58 and/or other than R at position 80; residues in NS1 including but not limited to other than M at position 117, other than K at position 176, and/or other than S at position 252, a nucleotide other than a at position 39, or any combination thereof. In one embodiment, the recombinant influenza B virus has an amino acid that results in enhanced replication in MOCK cells, e.g., in HA1, residue I at position, 34, residue E at position 129, E, 0 at position 168, P, isoleucine (1), A or N at position 196, or any combination thereof; in NA, residue T at position 169 and/or residue E at position 434; in NP, residue T at position 28, residue S at position 40, residue Q at position 51, residue K at position 52, residue G at position 57, residue T at position 214, g at nucleotide position 1795, or any combination thereof; in M1 residue valine (V) or N at position 34, residue G at position 54, residue K at position 77, residue T at position 86, residue N at position 97, or any combination thereof; in BM2 include residue R at position 58 and/or residue G at position 80; in NS1 include residue tyrosine (Y) at position 117, residue glutamine (Q) at position 176, residue T at position 252, a g at nucleotide position 39, additional g after nucleotide position 38, or any combination thereof.

In one embodiment, the recombinant or reassortant influenza B virus has an amino acid that results in enhanced replication in Vero cells, e.g., residues in HA1 including but not limited to a residue other than R at position 98, other than N at position 194, and/or other than T at position 196, and/or in HA2 including but not limited to a residue other than K at position 39, other than S at position 56, other than K at position 61, or other than D at position 112, or any combination thereof; residues in NA including but not limited to a residue other than residue T at position 76, other than residue R at position 102, other than residue E at position 105, other than residue P at position 139, other than residue T at position 436, other than D at position 457, or any combination thereof; residues in NP including but not limited to a residue other than P at position 343; residues in M1 other than G at position 34, other than I at position 97, or any combination thereof; residues in BM2 other than H at position 58, other than R at position 80, other than H at position 27, other than G at position 26, or any combination thereof; residues in NS1 including but not limited to a residue other than Y at position 42; residues in PA other than Y at position 387, other than V at position 434, other than D at position 494, other than T at position 524, a nucleotide other than a at nucleotide position 2272, a nucleotide other than g at nucleotide position 2213, a residue in PB2 other than N at position 16; or any combination thereof. In one embodiment, the recombinant influenza B virus has an amino acid that results in enhanced replication in Vero cells, e.g., in HA, P, I, A or N at position 196 (in HA1), residue K at position 98 (in HA1), residue D at position 194 (in HA1), residue G at position 39 (in HA2) residue G at position 56 (in HA2), residue N at position 51 (in HA2), or residue E at position 112 (in HA2), or any combination thereof; in NA residue M at position 76, residue K at position 102, residue K at position 105, residue S at position 139, residue M at position 436, and/or residue N at position 457, or any combination thereof; in NP residue T at position 343; in M1 include residue V or N at position 34 and/or residue N at position 97; in BM2 residue R at position 58, residue G at position 80, residue R at position 27, residue R at position 26, or any combination thereof; in NS1 residue N at position; in PA residue H at position 387, residue A at position 434, residue N at position 494, residue A at position 534, g at nucleotide position 2272, a t at nucleotide position 2213; residue S at position 16 in PB2; or any combination thereof.

In one embodiment, for viruses related to B/Yamagata-lineage, the recombinant or reassortant influenza B virus has an amino acid in HA1 other than K at position 129, other than N at position 168, other than N at position 194, other than T at position 196, other than 0 at position 112, or any combination thereof; in NA other than T at position 76, other than R at position 102, other than E at position 105, other than P at position 139, other than G at position 434, other than T at position 436, other than D at position 457, or any combination thereof; in NP other than E at position 52, other than S at position 57, other than P at position 343, or any combination thereof; in M1 other than G at position 34, other than R at position 77, other than I at position 97, or in NP vRNA, a nucleotide other than c a position 500, or any combination thereof; in BM2 other than H at position 58, other than R at position 80, other than H at position 27, other than G at position 26, or any combination thereof; in NS1 other than M at position 117, other than S at position 252, and/or other than D at position 494 in PA, and/or a nucleotide other than a at position 2272, other than g at nucleotide position 2213, other than a at position 1406, and/or other than c at position 1445 in PA vRNA, or any combination thereof; in PB2 a residue other than N at position 16; or any combination thereof. In one embodiment, the recombinant influenza B virus has in HA1 E at position 129, D at position 168; P at position 196, D at position 194, in HA2 at position 112, or any combination thereof; in NA M at position 76, K at position 102, K at position 105, 5 at position 139, E at position 434, M at position 436, and/or N at position 457, or any combination thereof; in NP K at position 52, G at position 57, T at position 343, or any combination thereof; in M1 V or N at position 34, K at position 77, N at position 97, or any combination thereof; in BM2 R at position 58, G at position 80, R at position 27, R at position 26, or any combination thereof; in NS1 Y at position 117, T at position 252, or any combination thereof; in PA in N at position 494, tat position 2272, a at position 2213; in PB2 S at position 16; or any combination thereof.

In one embodiment, for influenza B viruses that are related to B/Victoria-lineage, the recombinant or reassortant influenza B virus has an amino acid in HA1 other than T at position 34, other than R at position 98, other than T at position 196, and/or in HA2 a residue other than K at position 39, other than S at position 56, other than K at position 61, or any combination thereof; in NA other than N at position 169 and/or other than D at position 457; in NP other than A at position 28, than P at position 40, other than P at position 51, other than M at position 204, or in NP vRNA a nucleotide other than g at position 1795 or other than c at position 500, or any combination thereof; in M1 other than D at position 54 and/or other than M at position 86; in BM2 other R at position 80; in NS1 other than Y at position 42, other than K at position 176, nucleotide other than a at position 39; in PA other than Y at position 387, other than V at position 434, other than T at position 524, or in PA vRNA a nucleotide other than a at nucleotide position 2272, a nucleotide other than g at nucleotide position 2213, a nucleotide other than a at position 1406, a nucleotide other than c at position 1445, or any combination thereof. In one embodiment, the recombinant influenza B virus has an amino acid has in HA I at position 34, P, I, A or N at position 196, K at position 98, and in HA2 G at position 39, residue G at position 56, residue N at position 61, or any combination thereof; in NA T at position 169 and/or N at position 457; in NP T at position 28, S at position 40, Q at position 51, T at position 204, a at position 1795, or any combination thereof; in M1 G at position 54 and/or T at position 86; in BM2 G at position 80; in NS1 N at position 42; Q at position 176, g at position 39, an additional g after position 38; in PA H at position 387, A at position 434, A at position 534, t at position 2272, a at position 2213, or any combination thereof.

In one embodiment, the recombinant or reassortant influenza B virus has one or more, e.g., 2, 3, 4, 5, 6, 7, 8, 9, 10 or more of the following: a residue other than Y at position 387, other than V at position 434, other than D at position 494, other than T at position 524, a nucleotide other than a2272, g2213, a1406, c1445, or any combination thereof, in PA or PA vRNA, e.g., 2272t, 2213a, 1406g, 1445t, 387H, 434A, 494N, 524A, or any combination thereof (e.g., residue H at position 387, residue A at position 434, residue N at position 494, residue A at position 524, or any combination thereof); a residue other than T at position 34, other than R at position 98, other than K at position 129, other than N at position 168, other than N at position 194, and/or other than T at position 196, in HA1, other than K at position 39, other than S at position 56, other than K at position 61, or other than D at position 112, in HA2, e.g., in HA1 34I, K129E, 168D, 196P/I/A/N, 98K, or 194D, and in HA2 including 39G, 56G, 61 N, or 112E, or any combination thereof; a residue other than residue T at position 76, other than residue R at position 102, other than residue E at position 105, other than residue P at position 139, other than residue N at position 169, other than G at position 434, other than residue T at position 436, and/or residue D at position 457 in NA, e.g., 169T, 434E, 76M, 102K, 105K, 139S, 436M, 457N, or any combination thereof; a residue other than H at position 58, other than R at position 80, other than H at position 27, other than G at position 26 in BM2, such as residue R at position 58, residue G at position 80, residue R at position 27, and/or residue R at position 26; other than A at position 28; other than P at position 40, other than P at position 51, other than E at position 52, other than S at position 57, other than M at position 204, and/or other than P at position 343, and/or g at position 1795, or any combination thereof, in NP, e.g., residue T at position 28, residue S at position 40, residue Q at position 51, residue K at position 52, residue G at position 57, residue T at position 214, residue T at position 343, or any combination thereof; residue other than G at position 34, other than D at position 54, other than R at position 77, other than M at position 86, other than I at position 97, or any combination thereof in M1, e.g., residue V or N at position 34, residue G at position 54, residue K at position 77, residue T at position 86, and/or residue N at position 97; in PB2 other than N at position 16. In one embodiment, the recombinant virus has M1 34V/I97N, BM2 58R/80G, NP 40S, NS1 86T.

In one embodiment, the influenza virus of the invention is a recombinant or reassortant influenza virus having two or more of selected amino acid residues at specified positions in one or more segments for PA, NP, M (M1 and BM2), and/or NS, which can be employed with HA and NA genes of interest. In one embodiment, the recombinant reassortant influenza virus has two or more of in NP, A28T, P40S, P51Q, E52K, S57G, M204T, and/or P343T, and/or g1795a; in M1, G34V/N, D54G, R77K, M86T, 197N; in BM2, H58R, R80G, H27R, G26R; in NS1, M117Y, K176Q, S252T, a39g, an additional g after 38, Y42N, in PA, a2272t, g2213g, Y387H, V434A, D494N, T524A: in PB2 N16S.

In one embodiment, the influenza virus of the invention is a recombinant or reassortant influenza virus having two or more of selected amino acid residues at specified positions in one or more of PA, BM2, NP, M1, and/or NS1, which can be employed with HA and NA genes of interest. For example, in one embodiment, the recombinant influenza B virus has M1 34V/I97N, BM2 58R/80G, NP 40S, M1 M86T, or has NP P40S or has NP E52K, or has a substitution in NP, M1, and optionally NS1 and BM2, as in clones 1-8 in Table 3 and substitutions in NP, M1, and optionally NS1, as in clones 1-8 in Table 4.

In one embodiment, the influenza virus of the invention is a recombinant or reassortant influenza virus having two or more of a1406g, c1445t, a2272t in PA vRNA, P40S or P40S/M204T in NP, c500t in NP vRNA, M77K or M86T, and a39g or 38(+1)g in NS vRNA, or K176 Q in NS, e.g., an influenza virus having PA a1406g/c1445t/a2272t, NP P40S, c500t, M R77K and NS a39g K176Q, or having PA a1406g/c1445t/a2272, NP P40S/M204T, c500t, M M86T and NS 38(+1)g.

The invention will be described by the following nonlimiting examples.

### Example 1

The yield of vaccine viruses is important from an economic point of view. Even more important, the ability to produce high numbers of vaccine doses under tight timelines may save many lives during a virus outbreak. Mutations that increase the replicative ability of viruses in cell culture and/or embryonated chicken eggs are useful to amplify influenza viruses, and to establish robust influenza vaccine platforms. Currently, most influenza vaccines are generated in embryonated chicken eggs. Influenza vaccines generated in MDCK cells are now approved for human use in the U.S. and in Europe, and influenza vaccines derived from Vero cells are approved for human use in Europe.

To develop a high-yield influenza B virus backbone for growth of vaccine virus in these specific host cells, random mutagenesis of the internal genes of BYamagata/1/73 was conducted; the HA and NA genes of the mutant virus libraries were derived from BYokohama/UT-K31/2012 (Yamagata-lineage) or B/Yokohama/UT-K1A/2011 (Victoria-lineage), representing the two major influenza B virus lineages. The virus libraries that were generated possessed random mutations in the ‘internal’ viral genes as well as those encoding the viral surface glycoproteins hemagglutinin (HA) and neuraminidase (NA), as well as non-coding mutation. Those that conferred improved growth, and this are vaccine virus candidates, were further evaluated. In particular, these vaccine virus candidates confer higher yield in commonly used propagation systems for influenza vaccine virus production: that is, embryonated chicken eggs, Madin-Darby canine kidney cells, and African green monkey (Vero) cells. These vaccine candidates could be used to improve the influenza B virus vaccine production process.

### Materials and Methods

Cells. MDCK cells were grown in MEM containing 5% (vol/vol) newborn calf serum. Vero cells were maintained in MEM containing 10% (vol/vol) FBS. 293T human embryonic kidney cells were grown in DMEM supplemented with 10% (vol/vol) FBS.

Construction of Plasmids. The sequences of the eight viral RNAs of B/Yamagata/1/73 virus were used to design gBlocks Gene Fragments (Integrated DNA Technologies), which were amplified and joined by PCR; the resulting viral cDNAs were inserted into the RNA polymerase I vector pHH21 (Neumann et al., 1999). The vRNAs of the BYokohama/UT-K31/2012, BNokohama/UT-K1A/2011, B/Yokohama/P-2922/2005, B/Tokyo/UTE2/2008, B/Tochigi/UT-T1/2011, B/Massachusetts/2/2012, and B/Brisbane/60/2008 viruses were extracted from virus stocks by using the RNeasy Kit (Qiagen). The viral HA and NA genes were amplified with gene-specific oligonucleotides by using the One-Step RT-PCR Kit (Invitrogen), and the PCR-products were cloned into the pHH21 vector. The HA and NA genes of B/Yamagata/16/1988 were synthesized by PCR-amplification of joined gBlocks Gene Fragments, followed by cloning into pHH21. The type A/B chimeric HA and NA genes of B/Yokohama/UT-K31/2012, B/Yokohama/UT-K1A/2011, B/Massachusetts/2/2012 and B/Brisbane/60/2008 viruses were generated by overlapping PCRs.

Construction of Plasmid Libraries. One to four random mutations were introduced into each of the six internal genes of B/Yamagata/1/73 virus by error-prone PCR using the GeneMorph II Random Mutagenesis Kit. The randomly mutated PCR products were inserted into the pHH21 vector, and the diversity of the resulting plasmid libraries was confirmed by sequence analysis of at least 24 Escherichia coli colonies for each viral gene.

Virus Rescue and Virus Library Generation. Wild-type viruses and virus libraries possessing random mutations in the internal genes were generated with the help of reverse-genetics approaches (Neumann et al., 1999). Virus libraries were generated by transfecting 293T cells with a mutant plasmid library instead of the wild-type construct. Forty-eight hours later, supernatants from plasmid-transfected 293T cells were collected and amplified in MDCK cells to generate virus stock; the titers of the virus stocks were determined by using plaque assays in MDCK cells.

Evaluation of Viral Growth Kinetics. Wild-type or recombinant viruses were inoculated in triplicate into MDCK cells at a multiplicity of infection (MOI) of 0.001. After infection, cells were incubated with MEM/BSA medium with 0.6 μg/mL TPCK-trypsin. Supernatants were collected at the indicated time points and the virus titers were assessed by means of plaque assays in MDCK cells.

To analyze viral replication in embryonated chicken eggs, 10-day-old embryonated chicken eggs (four per virus) were inoculated with 1×10⁴ pfu of virus and incubated them at 35° C. The allantoic fluids were collected at the indicated time points and virus titers were determined by use of plaque assays in MDCK cells.

The hemagglutination titers of viruses amplified in MDCK cells or embryonated chicken eggs were determined by using a hemagglutination assay. Briefly, 50 μL of virus sample was serially diluted twofold in 96-well U-bottom microtiter plates (Thermo Scientific) containing 50 μL of PBS per well. Next, 50 μL of 0.5% turkey red blood cells were added to each well, plates were incubated for 45 minutes at room temperature, and hemagglutination titers were calculated as the reciprocal value of the highest dilution at which agglutination occurred.

Virus Concentration and Purification. MDCK cells were grown in two 4-Layers Easy-Fill Cell Factories (Thermo Scientific) and infected with wild-type or high yield influenza B viruses at an MOI of 0.001 when the cells reached about 95% confluency. Cell-culture supernatants were harvested 48 hours later and clarified by centrifugation (3,500 rpm in a Beckman SX4750 rotor, 15 minutes, 4° C.). Viruses were pelleted by ultracentrifugation (18,500 rpm, 90 minutes at 4° C. in a Beckman Type19 rotor), resuspended in 5 mL of PBS, and loaded onto 20-50% (wt/vol) continuous sucrose gradients which were centrifuged at 25,000 rpm for 90 minutes at 4° C. in a Beckman SW32 rotor. The virus-containing band was collected, diluted in PBS, and pelleted again by centrifugation (25,000 rpm, 90 minutes, 4° C., Beckman SW32 rotor). The virus pellet was resuspended in 400 μL of PBS, aliquoted, and stored at −80° C.

Total Protein Assay. Total protein yield of virus concentrates was determined by using the Pierce BCA protein assay kit (Thermo Scientific) according to the manufacturer's instructions.

Deglycosylation of Viral Proteins Using PNGase F. To remove sugar moieties, 10 μL of virus concentrate was denatured. The sample was then incubated at 37° C. for 20 hours with 2 μL of a one-tenth dilution of PNGase F enzyme (New England Biolabs) in the buffer provided by the manufacturer and with Nonidet P-40 at a final concentration of 1%.

SDS/PAGE. 2 μL of virus concentrate was mixed with PBS to a total volume of 10 μL and 2.5 μL of loading dye with 2% (vol/vol) β-mercaptoethanol (as reducing agent) was added, which mixture was heated to 95° C. for 5 minutes. Samples were then loaded onto NuPage 4-12% (wt/vol) Bris-Tris precast gels (Life technology), which were run at 150 V for 120 minutes using 1×Mes buffer (Bio-Rad), and then stained with SYPRO-Ruby (Sigma). Quantitation of protein amounts was carried out by using ImageJ software (NIH). The HA content was calculated by dividing the HA amount (calculated by summing the amounts of HA1 and HA2) by the sum of the amounts of HA1, HA2, NP, and M1, and multiplying this value by the amount of total viral protein.

Virulence Studies in Mice. Six-week-old female BALB/c mice (Jackson Laboratory) were anesthetized with isoflurane and inoculated intranasally with 106 pfu of influenza B viruses in a volume of 50 μL. Five mice were infected per group; this sample size is adequate to detect large effects between groups. Mice were randomized and investigators were not blinded. Body weight changes and survival were monitored daily for 14 days. To assess virus replication in mice, 10 mice per virus were infected with 106 pfu; on days 3 and 6 post-infection, five mice in each group were killed and virus titers in the lungs were determined by use of plaque assays in MDCK cells.

Genetic Stability Testing. To evaluate the genetic stability of the high-yield vaccine backbones, viruses possessing the HY(Yam) and HY(Vic) backbones combined with the HA and NA vRNAs of B/Yokohama/UT-K31/2012 and B/Yokohama/UT-K1A/2011, respectively, were passaged 10 times in MDCK cells at an MOI of 0.01. Viruses collected after each passage were sequenced by means of Sanger sequencing.

Minireplicon Assay. For the minireplicon assay, 293T cells and MDCK cells were transfected with 0.25 μg each of plasmids expressing the B/Yamagata/1/73 PB2, PB1, PA and NP proteins, together with 0.05 μg of pPoll-B/Yamagata/1/73-NS-Luc (which encodes the firefly luciferase gene under the control of the human RNA polymerase I promoter) or pPollC250-B/Yamagata/1/73-NS-Luc (which encodes the firefly luciferase gene under the control of the canine RNA polymerase I promoter), respectively. Cells were cotransfected with 0.025 μg of pGL4.74(hRluc/TK) (an internal control to monitor transfection efficiency; Promega). The transfected cells were incubated at 35° C. for 48 hours, lysed, and assayed for luciferase activity by using the dual-luciferase system detector kit according to the manufacturer's protocol (Promega). The firefly luciferase expression levels were normalized to the Renilla luciferase activity. The data presented are the averages of three independent experiments±SD.

To investigate the significance of the mutations in the noncoding regions of the B/Yamagata/1/73 PA and NS1 vRNAs, cells were transfected as described above; however, pPollC250-NP(0)Fluc(0) was replaced with a reporter construct in which the firefly luciferase gene was flanked by the wild-type or mutant noncoding regions of the PA or NS vRNAs, respectively. At 48 hours post-transfection, luciferase activity was measured as described above.

VLP Budding Assay. For the VLP budding assay, 293T cells were transfected with 2 μg each of protein expression plasmids for wild-type or mutant B/Yamagata/1/73 M1, HA, NA, NP, BM2, and NS2. At 48 hours post-transfection, culture supernatant was harvested, clarified, loaded on a 20% (wt/vol) sucrose cushion, and ultracentrifuged at 60,000 rpm for 2 hours in a Beckman SW 60 Ti rotor; the pelleted VLPs were then resuspended in PBS overnight at 4° C. In parallel, we lysed the transfected cells with RIPA buffer.

Purified VLPs and cell lysates were separately mixed with 5× loading dye buffer and fractionated on NuPage 4-12% (wt/vol) Bris-Tris precast gels (Life Technology). The proteins were transferred to nitrocellulose membranes by using an iBlot dry blotting system (Invitrogen). The membranes were then blocked for 3 hours at room temperature with PBS with 0.05% Tween 20 (PBS-T) containing 5% (wt/vol) skimmed milk. Then, the membranes were incubated with monoclonal antibodies to B/Brisbane/60/2008 HA (1:1,000; my BioSource, MBS430175), or to influenza B virus NP (1:1,000; Abcam, ab47876) or M1 (1:2,000; Abcam, ab82608) protein overnight at 4° C. After four washes with PBS-T for 10 minutes each, the membranes were incubated with goat antimouse secondary antibodies conjugated with horseradish peroxidase (1:2,000; Life Technology) for 1 hour at room temperature. After four washes with PBS-T for 10 minutes each, the blots were developed by using lumi-light Western blotting substrate (Roche Applied Science) and visualized following autoradiography. Quantitation of protein amounts was carried out by using ImageJ software (NIH). To calculate the percentages of HA, NP, and M1 protein incorporation into VLPs, the following formula was used: (ratio of the amount of protein in the mutant VLP to the total amount of mutant protein (VLP+cell lysate)/ratio of the amount of protein in the wild-type VLP to the total amount of wild-type protein (VLP+cell lysate))×100.

IFN Antagonist Assays. To assess the IFN-antagonist activity of wild-type and mutant NS1 proteins, 293T cells were transfected with the NS1 protein expression plasmid and the reporter plasmid pGL-IFN-β, which encodes the firefly luciferase protein under the control of the IFN-β promoter (Bale et al., 2012). Twenty-four hours post transfection, cells were infected with Sendai virus at an MOI of 5 for 1 hour. Cells were incubated for 24 hours and lysed with Glo lysis buffer (Promega); then, Steady-Glo assay buffer (Promega) was added and luciferase expression measured. In another set of experiments, 293T cells were transfected with wild-type or mutant NS1 protein expression plasmids and the reporter plasmid pISRE-Luc (Promega), which encodes the firefly luciferase protein under the control of an IFN-regulated promoter. Twenty-four hours later, cells were treated with 104 U/mL of human IFN-β for another 24 hours, followed by lysis and measurement of luciferase expression levels.

Statistical Analysis. Statistical analyses of the data were accomplished using the R software (www.r-project.org), v3.1. To compare multiple groups with measurements collected independently at several time points, a two-way ANOVA followed by Tukey's post hoc test was used. To compare measurements from multiple groups collected at a single time point, a one-way ANOVA followed by either Tukey's or Dunnett's post hoc test was used. To compare multiple groups with dependent measurements (e.g., viral growth curves in cell culture for which aliquots were collected from the same culture at different time points), a linear mixed-effects model to the data by using the R package NLME; the time, virus strains, and interaction between these two factors were considered. The R package PHIA was used to build a contrast matrix for comparing strains in a pairwise fashion at the same time point (e.g., group_1 vs. group_2 at 24 hours postinfection, group_1 vs. group_3 at 24 hours postinfection, group_2 vs. group_3 at 24 hours postinfection). Comparisons were performed individually; therefore, the final P values were adjusted by using Holm's method to account for multiple comparisons.

Raw data from growth curves were converted to the logarithmical scale before being analyzed; results were considered statistically significant for P (or adjusted P values)<0.05. Variance between groups was assessed by using Levene's test (which was similar for the groups being compared, with P>0.05).

Ethics and Biosafety. The experiments in mice followed the University of Wisconsin-Madison's Animal Care and Use Protocol. All experiments were approved by the Animal Care and Use Committee of the University of Wisconsin-Madison (protocol number V00806), which acknowledged and accepted both the legal and ethical responsibility for the animals, as specified in the Fundamental Guidelines for Proper Conduct of Animal Experiment and Related Activities in the Animal Welfare Act and associated Animal Welfare Regulations and Public Health Service Policy.

### Results

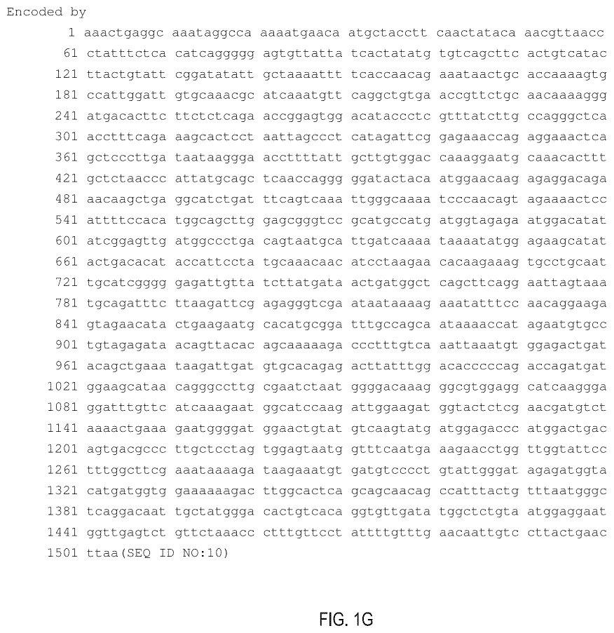

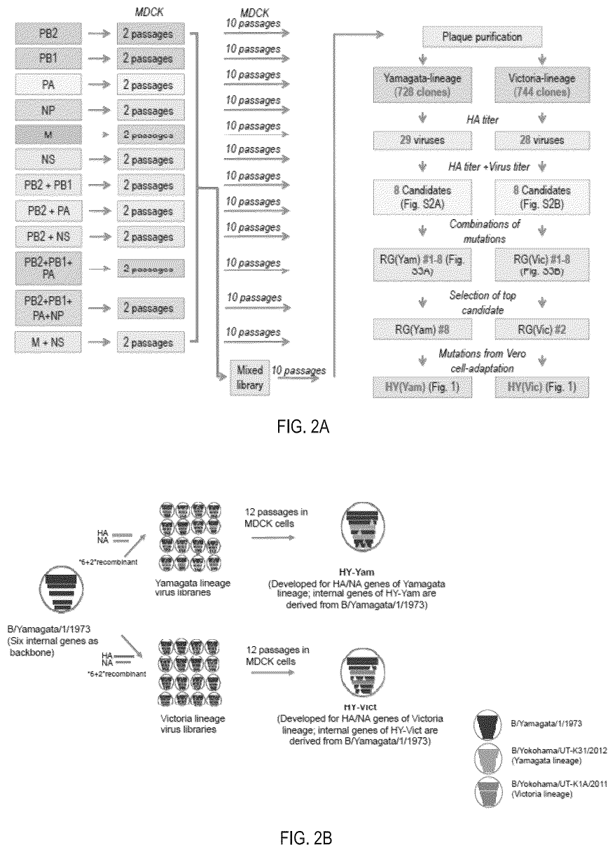

Virus Library Screens for High-Yield Variants in MDCK Cells. Comparable to a strategy to develop a high-yield influenza A virus PR8 vaccine backbone, mutagenesis and screening approaches were used to identify mutations associated with high yield of an influenza B virus. The six internal vRNA segments were from the B/Yamagata/1/73 virus, which grows efficiently in MDCK cells and was isolated before the Victoria and Yamagata lineages separated. A mutagenesis approach based on error-prone PCR was then used to generate libraries of cDNAs possessing one to four random amino acid changes in the viral proteins (FIG. 2). These cDNA libraries were then used to generate virus libraries. Six separate libraries representing each of the internal vRNAs (i.e., PB2, PB1, PA, NP, M, and NS) were generated (FIG. 2); three libraries for combinations of the polymerase vRNAs (i.e., PB2+PB1, PB2+PA, PB2+PB1+PA); one library for the polymerase and nucleoprotein (NP) vRNAs (i.e., PB2+PB1+PA+NP) because the PB2, PB1, PA, and NP proteins form the viral replication complex; one library for the PB2 and NS vRNAs (PB2+NS) because the PB2 and NS1 proteins (encoded by the NS vRNA) of influenza A viruses are important determinants of host virulence (Wright et al., 2013); and one library for the M and NS vRNAs because the M1 protein (encoded by the M vRNA) of influenza A viruses is associated with high-growth properties (Ramanunninair et al., 2013). Each of these 12 virus libraries was generated with the HA and NA vRNAs of a representative virus of the Victoria (B/Yokohama/UT-K1A/2011) or Yamagata (B/Yokohama/UTK31/2012) lineage, respectively, resulting in a total of 24 virus libraries. Libraries generated with the HA and NA vRNAs of the Victoria-lineage or Yamagata-lineage viruses will be referred to as “Victoria-lineage” or “Yamagata-lineage” libraries, respectively.

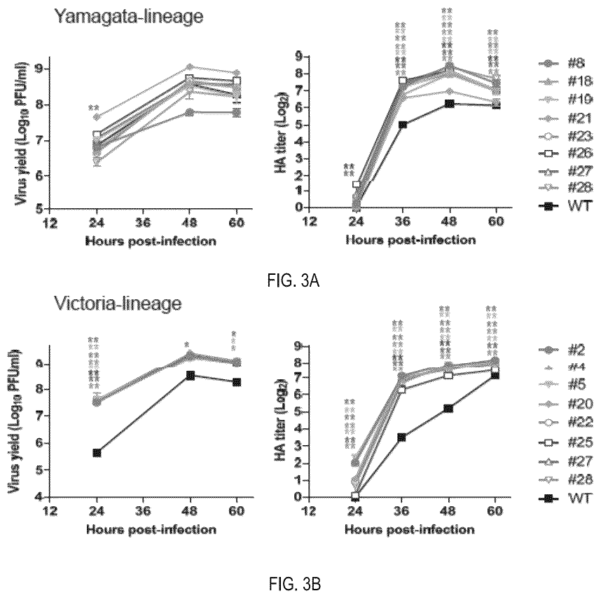

To select variants with enhanced growth properties, each library was passaged 12 times in MOCK cells. In parallel, virus libraries were combined after two passages in MDCK cells, and then 10 additional passages were performed in MDCK cells (FIG. 2). More than 700 viral plaques were randomly selected each from the Victoria- and Yamagata-lineage libraries, respectively, resulting in a total of 1,472 individual, plaque-purified viruses (FIG. 2). The plaque-purified viruses were then amplified in MDCK cells, and their yields were assessed in hemagglutination assays (as a surrogate for high HA yield) and compared with those of the parental Victoria- and Yamagata-lineage viruses (which possess the Victoria- or Yamagata-lineage lineage HA and NA vRNAs in combination with the six remaining vRNAs of B/Yamagata/1/73 virus). 29 Yamagata- and 28 Victoria-lineage viruses were identified with hemagglutination titers that were at least twofold higher than those of the respective control virus. These candidate viruses were reamplified in MDCK cells, and their high-yield properties were confirmed by assessing hemagglutination titers and replication kinetics in MDCK cells (used as another surrogate for high HA yield) (FIG. 3).

Next, the entire viral genomes of the top eight candidates of each lineage were sequenced and different sets of mutations were found for high-yield candidates of the Yamagata- and Victoria-lineages (amino acid changes and changes in the noncoding regions were evaluated) (Tables 1 and 2). Seven of the eight high-yield candidates isolated from the Yamagata-lineage libraries possessed G34V and 197N mutations in the M1 matrix protein, and H58R and R80G mutations in the BM2 ion channel protein (also encoded by the M gene) (Table 1), suggesting that these amino acid substitutions may confer efficient replication in MDCK cells.

All eight high-yield candidates obtained from the Victoria-lineage libraries encoded a P40S mutation in NP and an M86T mutation in M1 (Table 2). In addition, six of these eight high-yield candidates encoded nucleotide changes in the noncoding region of the NS vRNA: an additional nucleotide after position 38 was detected in five viruses (NS-38(+1)g; all nucleotide changes in noncoding regions are shown in italicized lowercase letters), and an a39g nucleotide replacement was detected in one virus (Table 2). A g1795a mutation was identified in the noncoding region of the NP segment in three high-yield candidates (Table 2).

Although the HA and NA vRNAs were not targeted by PCR-mediated random mutagenesis, several mutations were detected in the HA and NA proteins (Tables 1 and 2). Specifically, threonine at position 196 of HA was replaced with various other amino acids (e.g., alanine, isoleucine, proline, or asparagine) in seven of eight high-yield candidates derived from the Victoria-lineage libraries (Table 2), suggesting strong selective pressure at this position.

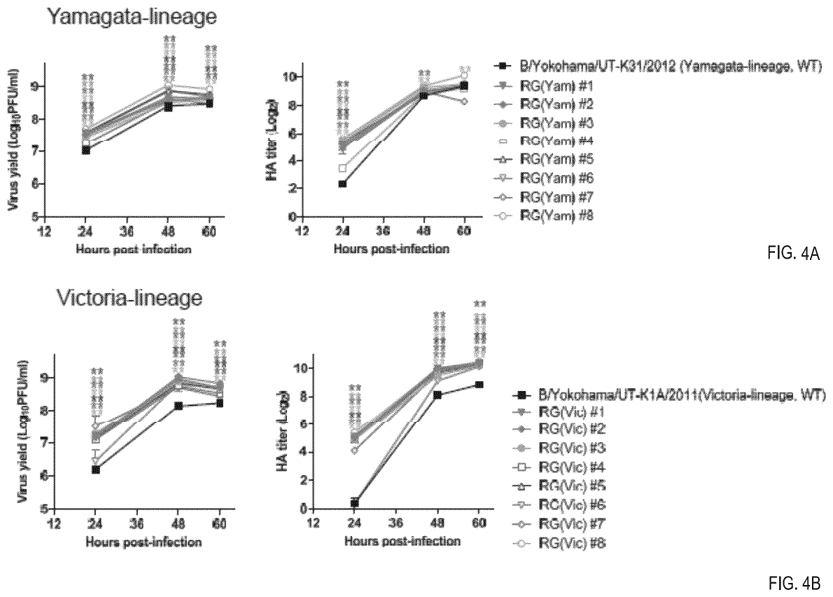

Potential Combinatorial Effects of Mutations. Next, reverse-genetics approaches were used to generate viruses possessing various combinations of the mutations found in the top eight high-yield Yamagata- or Victoria-lineage candidates (Tables 3 and 4). For example, the NP-E52K andM1-R77K mutations found in high-yield candidate #21 (which replicated to the highest titers in MDCK cells) (FIG. 3) were combined with the NS1-M117Y/S252T mutations found in high-yield candidates #23 and #26 (FIG. 3). Because the internal vRNAs of the Yamagata- and Victoria-lineage libraries are derived from the same virus, mutations found in high-yield Yamagata- and Victoria-lineage candidates were also combined (Tables 3 and 4). The resulting viruses were tested for their hemagglutination titers and viral titers in MDCK cells (FIG. 4). All high-yield Yamagata- and Victoria-lineage candidates replicated in MDCK cells more efficiently and had higher hemagglutination titers than the wild-type viruses at one or more time points, and most of these differences were statistically significant (FIG. 4).

Yamagata-lineage RG(Yam) #8 (encoding NP-P40S, M1-R77K, NS1-K176Q, and NS-a39g mutations) and Victoria-lineage RG(Vic) #2 (encoding NP-P40S/M204T, M1-M86T, and NS-38 (+1)g mutations) were selected as lead candidate vaccine backbones because they had the highest titers in their respective groups.

Virus Library Screens for High-Yield Variants in Vero Cells. The ideal vaccine virus backbone should confer a high yield in all three propagation systems currently used in the commercial production of human influenza vaccines: that is, MDCK cells, Vero cells, and embryonated chicken eggs. In parallel to the development of a high-yield influenza B vaccine backbone in MDCK cells, all 24 virus libraries (FIG. 2) were passaged in Vero cells. Because the titers of influenza B virus libraries were low in Vero cells, first the libraries were passaged in cocultured MDCK and Vero cells five times, followed by five passages in Vero cells. In parallel, virus libraries were combined after two passages in cocultured MDCK and Vero cells, passaged three more times in cocultured cells, and then passaged five times in Vero cells. A total of 382 individual virus plaques were randomly picked from the various virus libraries and amplified in Vero cells. Based on the results of hemagglutination assays, the top six candidates were picked from the Yamagata and Victoria lineage, Vero cell-passaged libraries and determined their full genomic sequences (Tables 5 and 6).

The high-yield candidates possessed several of the mutations that were identified after the MDCK cell passages, which may have been selected in the MDCK cells during the passages in cocultured cells. These mutations include amino acid changes at positions M1-34/97, BM2-58 or -80, and HA1-196 (compare Tables 1 and 2 with Tables 5 and 6). The mutations at positions M1-34/97 and BM2-58 were only detected in the Yamagata-lineages libraries, whereas mutations at position BM2-80 occurred in both the Yamagata- and Victoria-lineage viruses. The mutation at position HA1-196 predominated among viruses of the Victoria lineage, but also occurred in one virus of the Yamagata lineage. In addition, mutations not previously found after passages in MDCK cells were observed, most notably an a2272t nucleotide replacement in the noncoding region of PA, which was found in high-yield candidates of both virus lineages (Tables 5 and 6).

Selection of High-Yield Yamagata- and Victoria-Lineage Vaccine Virus Candidates. Next, it was tested whether the PA-a2272t mutation found after the Vero cell passages would enhance the growth properties of RG(Yam) #8 and/or RG(Vic) #2. The resulting viruses (HY(Yam) and HY(Vic), respectively) displayed higher hemagglutination and virus titers in MDCK cells compared with RG(Yam) #8 and RG(Vic) #2, and compared with the parental Yamagata- and Victoria-lineage viruses; some of these differences were small (although statistically significant). Therefore, HY(Yam) (encoding NP-P40S, M1-R77K, NS1-K176Q, NS-a39g, PA-a2272) and HY(Vic) (encoding NP-P40/M204T, M1-M86T, NS-(38+1)g, PA-a2272t) were selected as lead high-yield candidates.

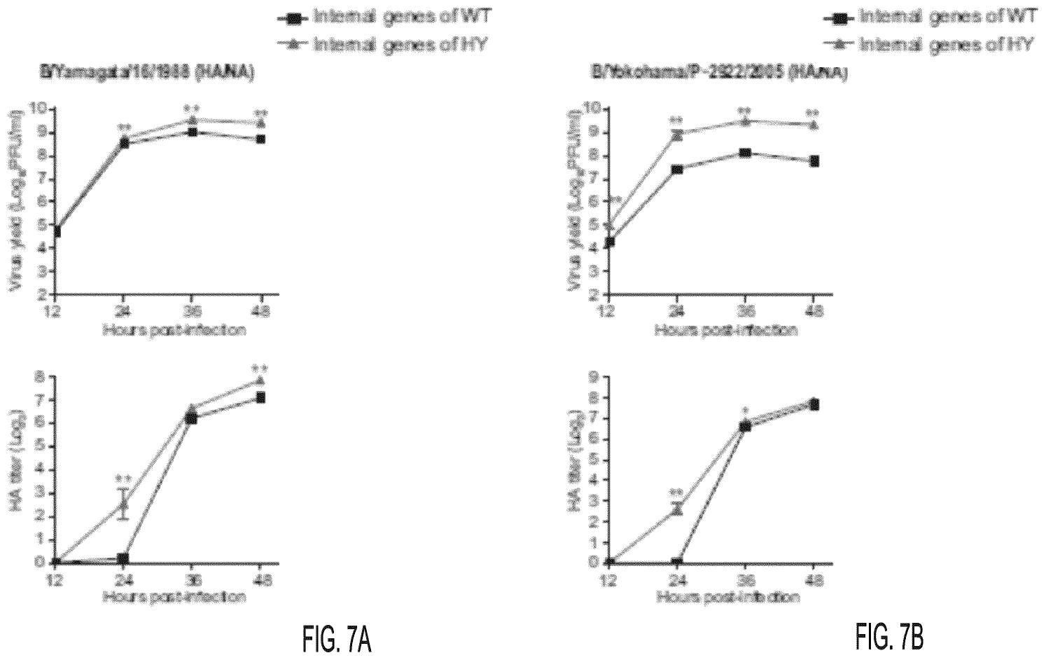

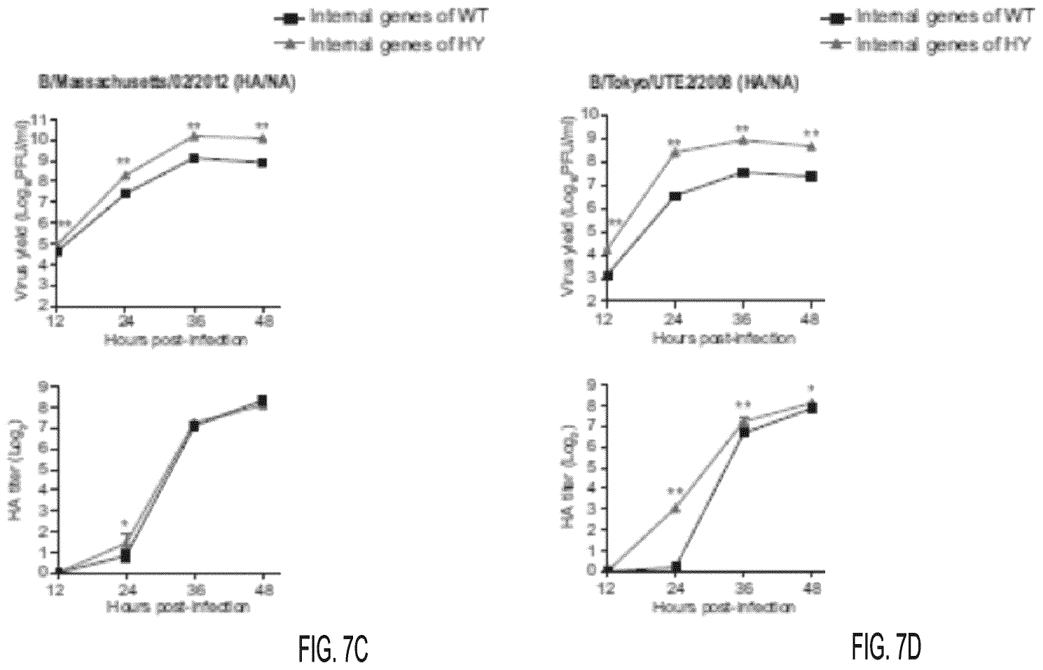

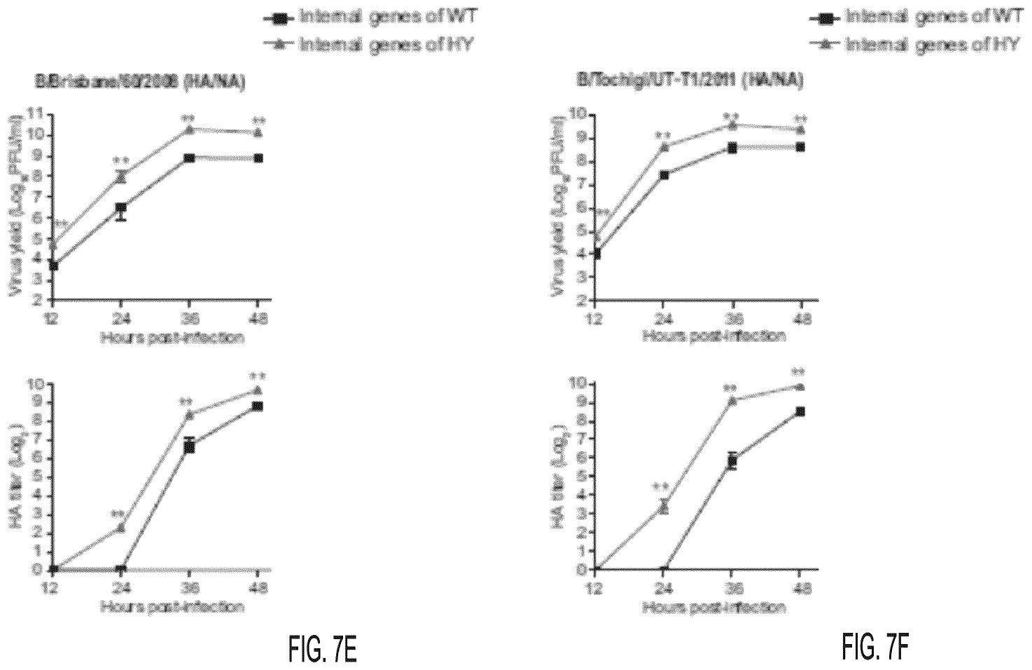

Evaluation of High-Yield Vaccine Virus Backbones with Different Influenza B Virus HA and NA Genes. HY(Yam) and HY(Vic) were developed with the HA and NA genes of B/Yamagata/UT-K31/2012 and B/Yokohama/UT-K1A/2011, respectively. Because high-yield vaccine virus backbones should have a general growth-enhancing effect, the HY(Yam) and HY(Vic) vaccine virus backbones with the HA and NA genes of six influenza B viruses isolated over several decades, including WHO-recommended vaccine viruses, were tested (FIG. 7). At one or more time points tested, the HY(Yam) and HY(Vic) vaccine virus backbones conferred higher viral or hemagglutination titers compared with the parental viruses, although not all of the differences were statistically significant. For the viruses tested, HY(Vic) had a greater growth-enhancing effect than HY(Yam).

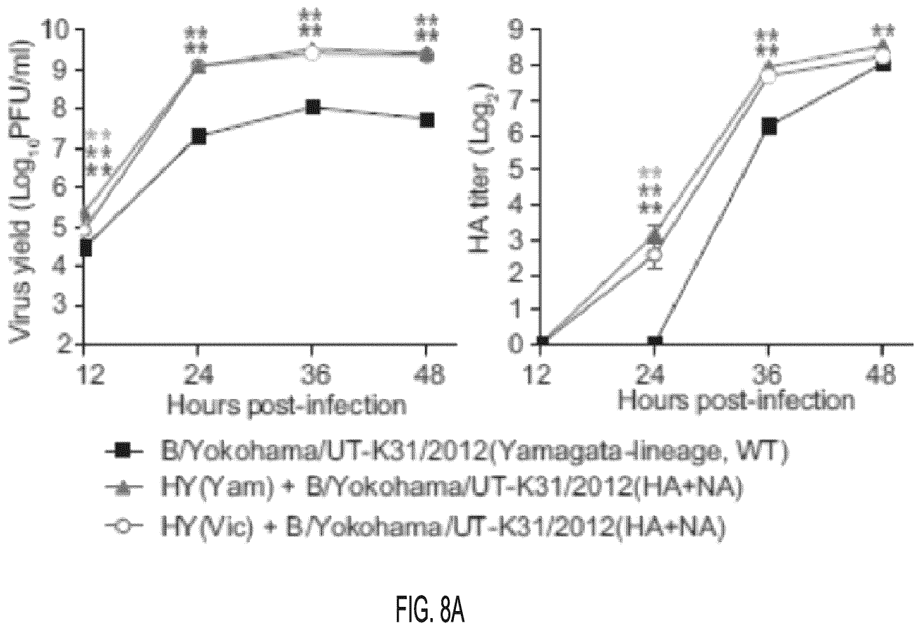

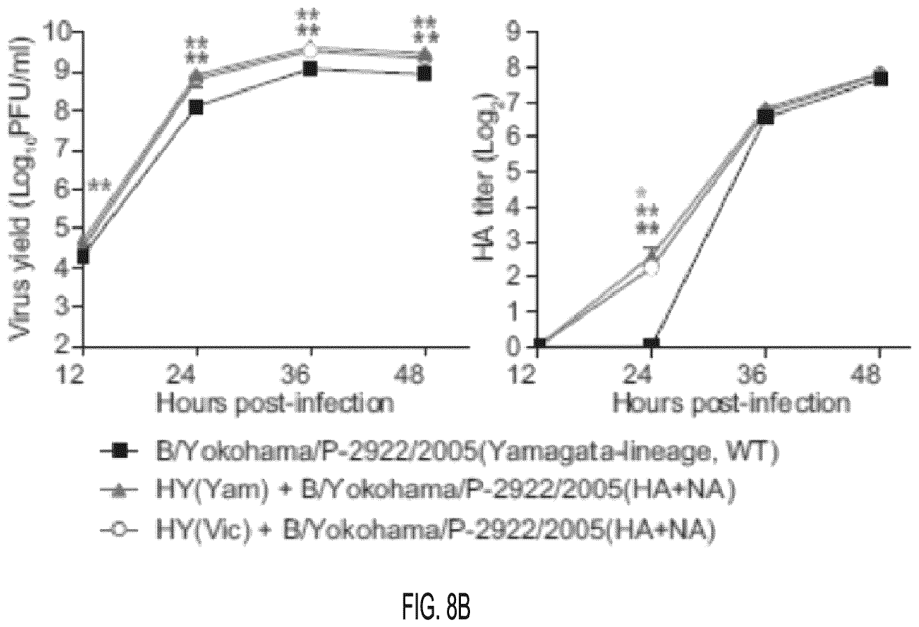

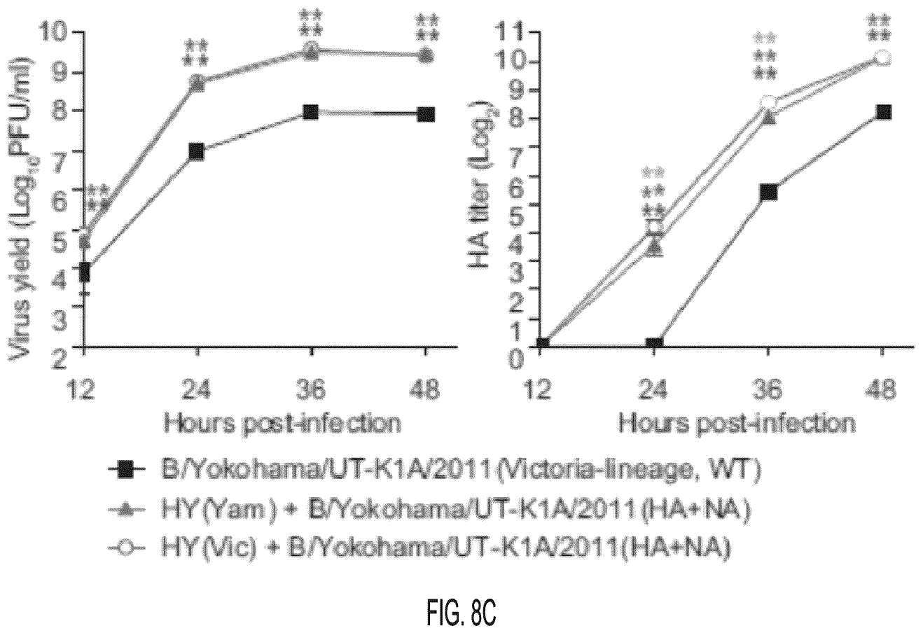

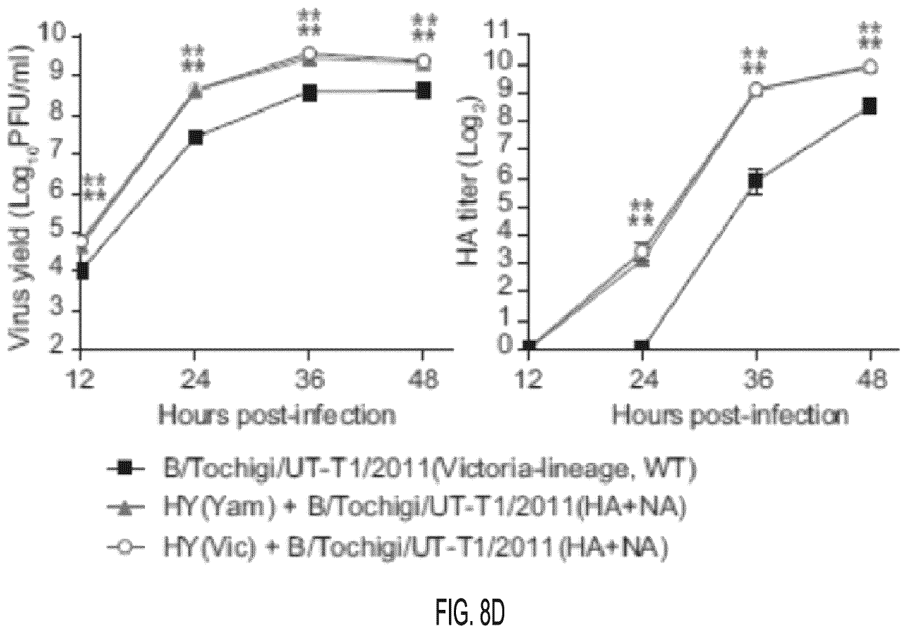

Exchange of HY(Yam) and HY(Vic) Backbones. Next, it was tested whether the HY(Yam) and HY(Vic) backbones supported efficient replication of viruses possessing HA and NA genes derived from the other influenza B virus lineage (FIG. 8). High viral and hemagglutination titers were detected for HY(Yam) viruses encoding Victoria-lineage HA and NA genes, and for HY(Vic) viruses encoding Yamagata-lineage HA and NA genes. Overall, the HY(Yam) vaccine backbone resulted in slightly higher virus or hemagglutination titers than the HY(Vic) vaccine backbone at several time points, but most of these differences were not statistically significant. These data indicate that the HY(Yam) and HY(Vic) vaccine backbones confer efficient replication to viruses of both lineages.

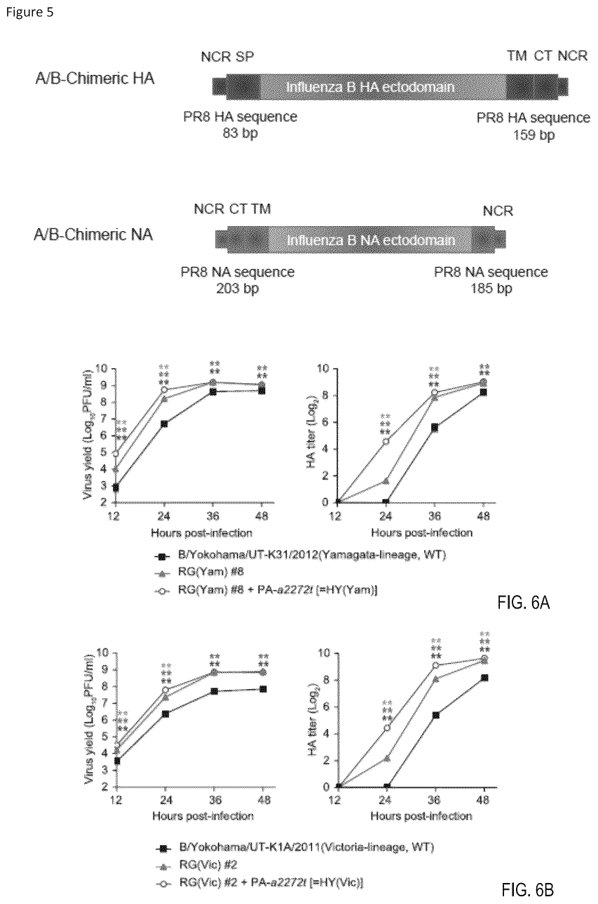

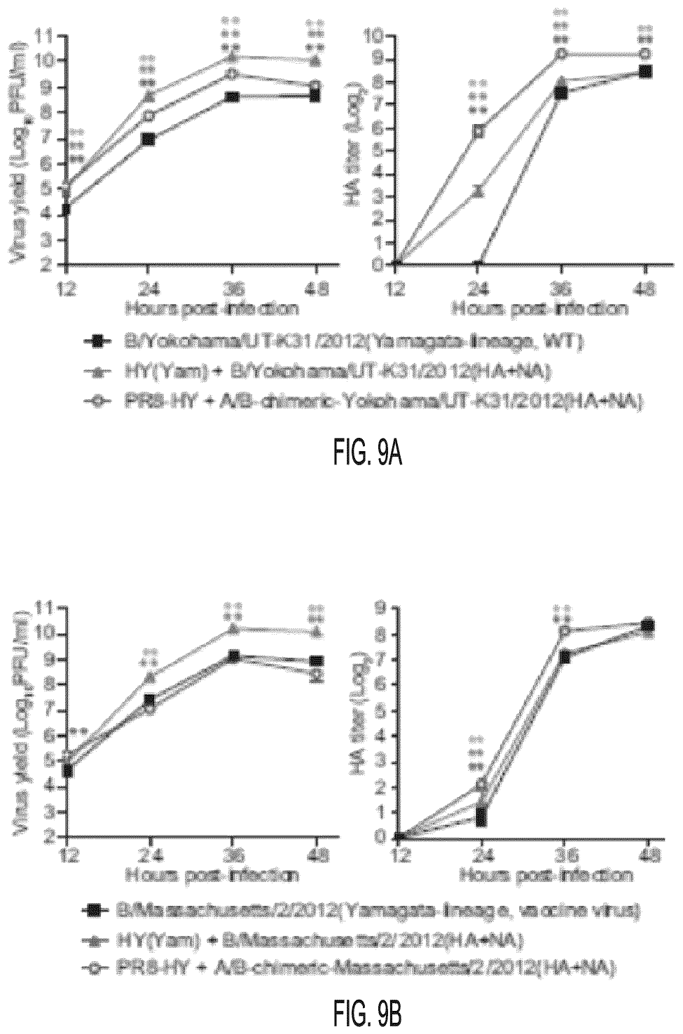

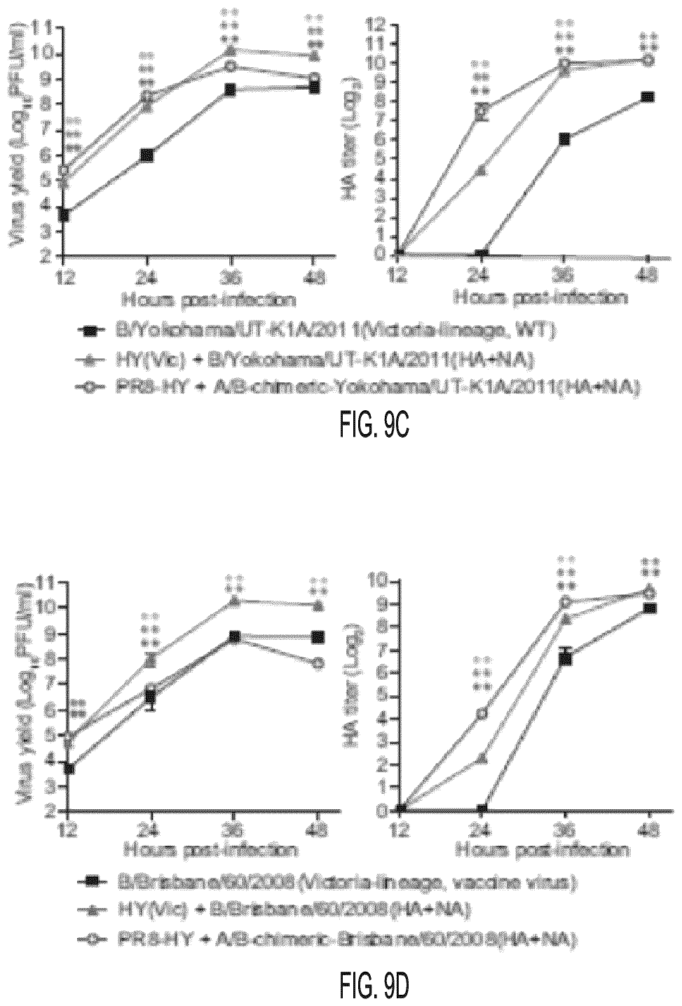

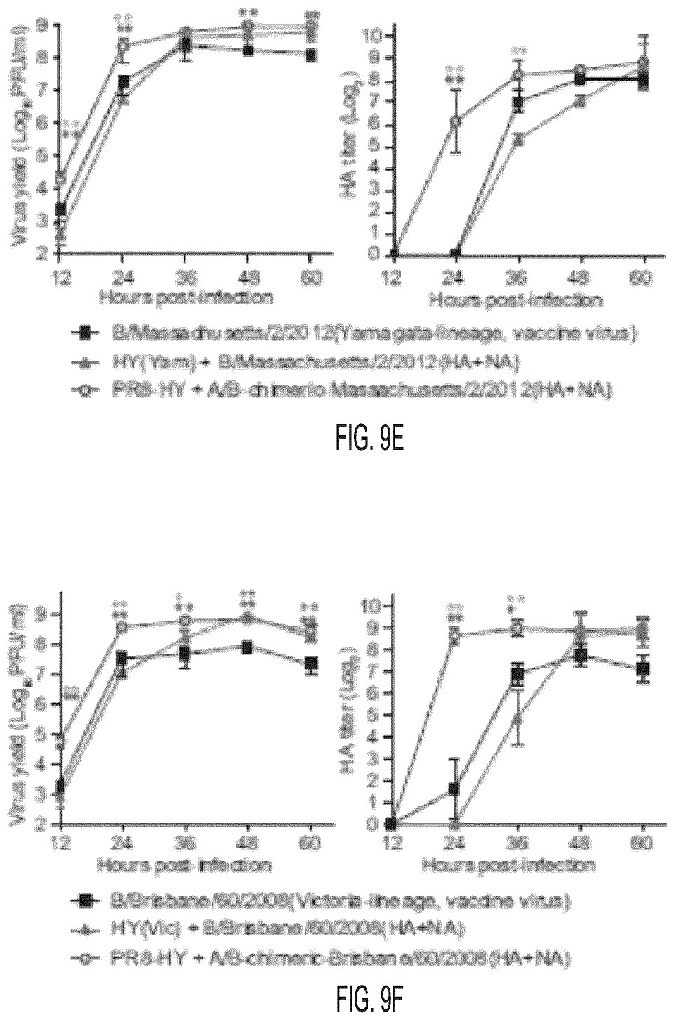

Comparison of Influenza A and B Virus Vaccine Backbones. Chimeric influenza A viruses possessing the HA and NA genes of an influenza B virus have been generated previously (Horimoto et al., 2004 and Flandorfer et al., 2003). The use of a universal backbone for both influenza A and B viruses would simplify the vaccine production process. Reassortants between influenza A and B viruses do not occur naturally, likely because of type-specific viral packaging signals located in the 5′ and 3′ terminal regions of influenza vRNA segments (Fujii et al., 2003; Baker et al., 2014). Therefore, vRNA segments were generated in which the ectodomains of the influenza A PR8 virus HA and NA proteins were replaced with influenza B virus counterparts (FIG. 5). Then the hemagglutination and viral titers of the following three viruses were compared: a wild-type Yamagata-lineage influenza B virus; HY(Yam) with the HA and NA genes of a Yamagata-lineage virus; and high-yield PR8 virus with type A/B chimeric HA and NA genes of a Yamagata-lineage virus (FIGS. 9A-B). Similar experiments were carried out for viruses of the Victoria-lineage (FIGS. 9C-D). At most time points tested, the high-yield influenza A or B vaccine virus backbones conferred significantly increased hemagglutination and viral titers compared with wild-type viruses. Comparison of the influenza A and B vaccine backbones revealed higher virus titers with the influenza B vaccine backbone but, interestingly, higher hemagglutination titers with the influenza A vaccine backbone. Moreover, for two influenza B viruses representing both lineages, the influenza A vaccine backbone was superior to the influenza B vaccine backbones with respect to hemagglutination and viral titers in embryonated chicken eggs (FIGS. 4E-F). Other influenza B viruses containing the HA and NA from B/Yokohama/UT-K1A/2011, B/Yokohama/P-2922/2005, B/Tokyo/UTE2/2008, or B/Tochigi/UT-T1/2014 did not grow well in embryonated chicken eggs regardless of the backbone (wild-type or high-yield), suggesting that the HA and NA genes of these human viruses may restrict efficient growth in embryonated chicken eggs.

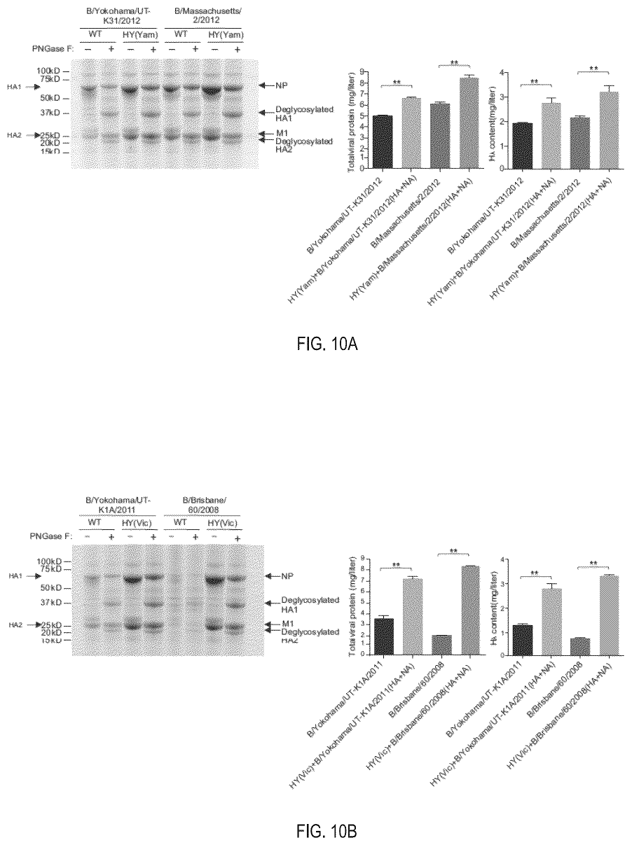

Evaluation of Total Viral Protein Yield and HA Content. Most preparations of inactivated influenza vaccines contain 15 μg each of H1 HA, H3 HA, and type B HA proteins. For vaccine optimization, total viral protein yield and HA content are therefore important parameters, prompting us to compare the total viral protein yield and HA content of different HY(Yam) and HY(Vic) viruses with their respective wild-type viruses in MDCK cells. Cell-culture supernatants were collected from infected cells, and viruses were concentrated and purified by use of sucrose gradient centrifugation. The total viral protein yield was then determined by using the Pierce BCA Protein Assay Kit (Thermo Scientific). In parallel, purified virus samples were treated with PNGase F, resulting in HA deglycosylation, which allows easier detection of HA2 (which in its glycosylated form is similar in size to M1). Samples were separated by using SDS/PAGE (FIGS. 10A-B) and the amounts of HA1, HA2, NP, and M1 were determined based on densitometric analysis. The HA content was calculated by dividing the HA amount (calculated by summing the amounts of HA1 and HA2) by the sum of the amounts of HA1, HA2, NP, and M1, and multiplying this value by the amount of total viral protein in the samples analyzed via gel electrophoresis (FIGS. 10A-B). For all viruses tested, total viral protein yield and HA content were significantly higher with the HY(Yam) and HY(Vic) vaccine backbones compared with the wild-type viruses from which the HA and NA vRNAs were derived.

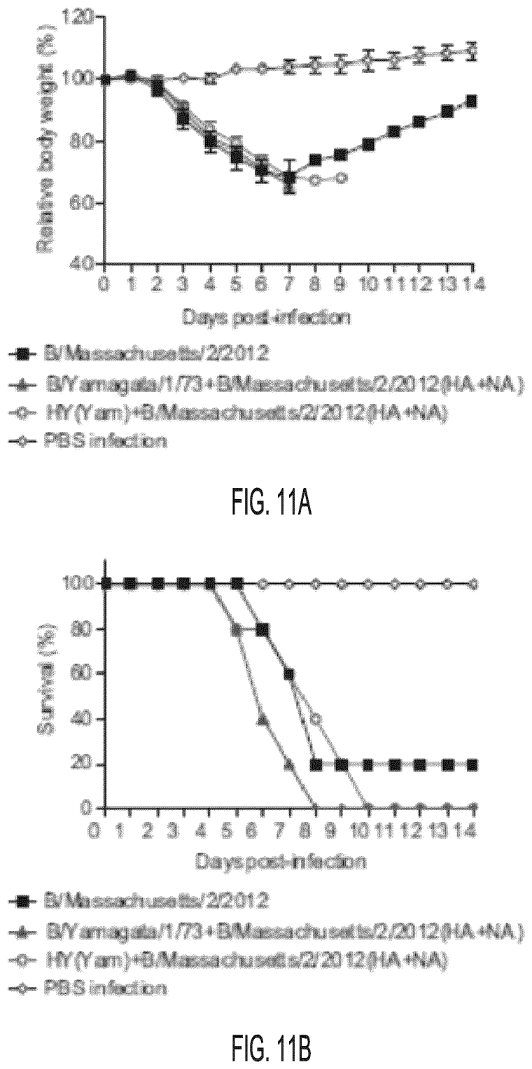

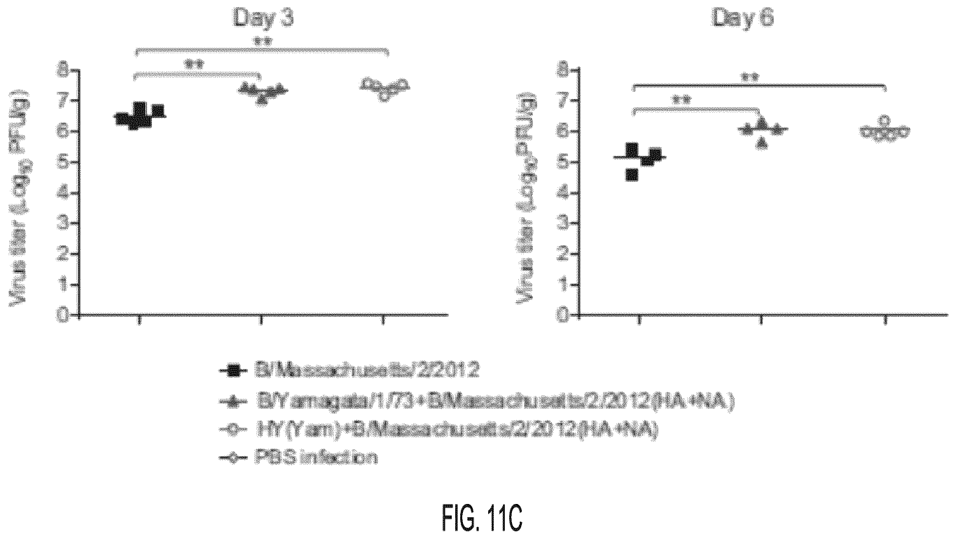

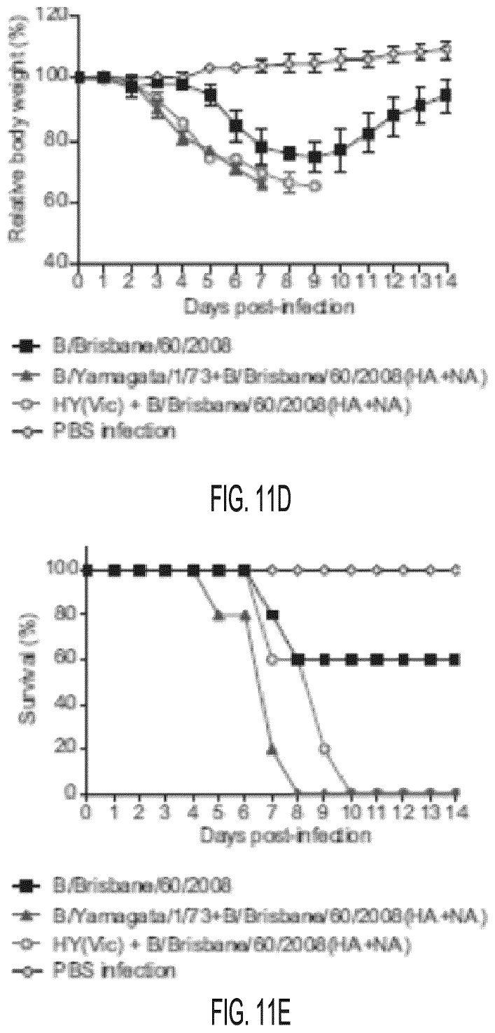

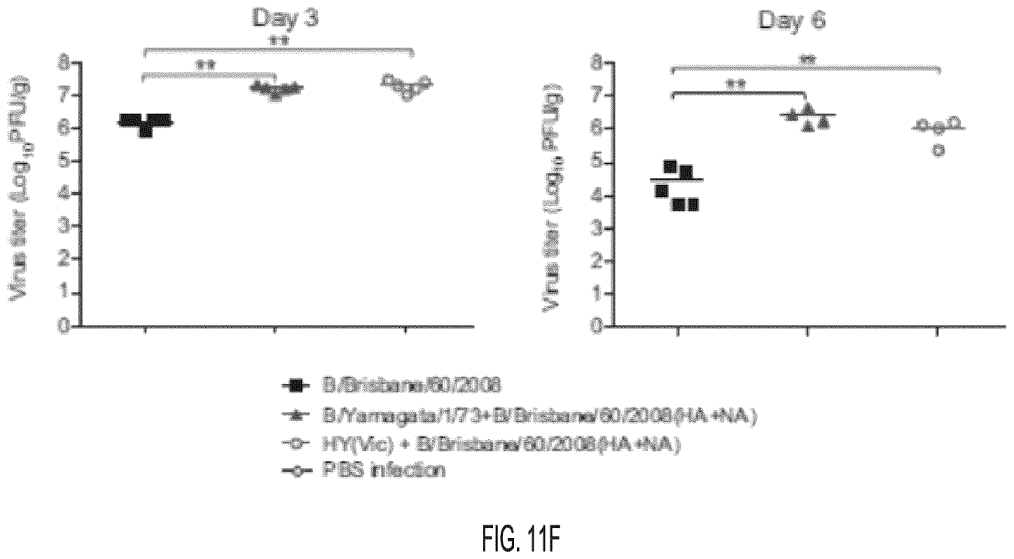

Virulence of PR8-HY-Based Vaccine Viruses in Mice. An experimental approach was designed to select mutants with increased replicative ability, which may also increase their virulence in mammals. To address this question, five mice per group were infected with 106 pfu of a wild-type Yamagata-lineage virus, a virus possessing the HA and NA vRNAs of the Yamagata-lineage virus in combination with the remaining six genes of wild-type B/Yamagata/1/73 virus (which was used to generate the virus libraries), and a virus possessing the HA and NA vRNAs of the Yamagata-lineage virus in combination with the HY(Yam) vaccine virus backbone (FIGS. 11A-B). In parallel, groups of 10 mice were infected with 10⁶ pfu of the viruses described above, and five animals each were killed on days 3 and 6 post-infection to assess lung virus titers (FIG. 11C). Similarly, experiments were carried out with a Victoria-lineage viruses and the HY(Vic) vaccine backbone (FIGS. 11D-F). The wild-type B/Yamagata/1/73 virus backbone conferred higher pathogenicity than the B/Massachusetts/2/2012 and B/Brisbane/60/2008 backbones, respectively. However, the yield-enhancing mutations of HY(Yam) and HY(Vic) did not increase mouse virulence further; in fact, they had slightly attenuating effects compared with the B/Yamagata/1/73 backbone.

Genetic Stability of the HY(Yam) and HY(Vic) Vaccine Backbones. Vaccine viruses should be genetically stable so that their desired properties are maintained. To test the genetic stabilities of the high-yield candidates, 10 serial passages of a HY(Yam) virus were performed with the HA and NA vRNAs of B/Yokohama/UTK31/2012, and of a HY(Vic) virus with the HA and NA vRNAs of B/Yokohama/UT-K1A/2011 in MDCK cells. After each passage, the genomic sequences of the viruses were determined by Sanger sequencing. For the Yamagata-lineage virus, no mutations were detected. For the Victoria-lineage virus, mutations in the internal genes that define the high-yield properties of HY(Vic) were not detected. However, a mixed population encoding HA1-196T and -1961 was detected after passage 5; after passage 10, only the HA1-1961 mutant was detected.

Similarly, several wild-type and high-yield influenza B viruses of both lineages were passaged in embryonated chicken eggs (Table 7). After 5-10 consecutive passages, no egg-adapting mutations were detected in the internal genes. However, for viruses that possessed a glycosylation site at amino acids 194-196 of HA, mutations arose that resulted in the loss of that glycosylation site. This finding is consistent with the earlier finding of mutations at this glycosylation site in viruses of the Victoria-lineage (Table 2). Collectively, the data indicate that the yield-enhancing mutations in HY(Yam) and HY(Vic) were genetically stable for at least 5-10 consecutive passages in MDCK cells and embryonated chicken eggs.

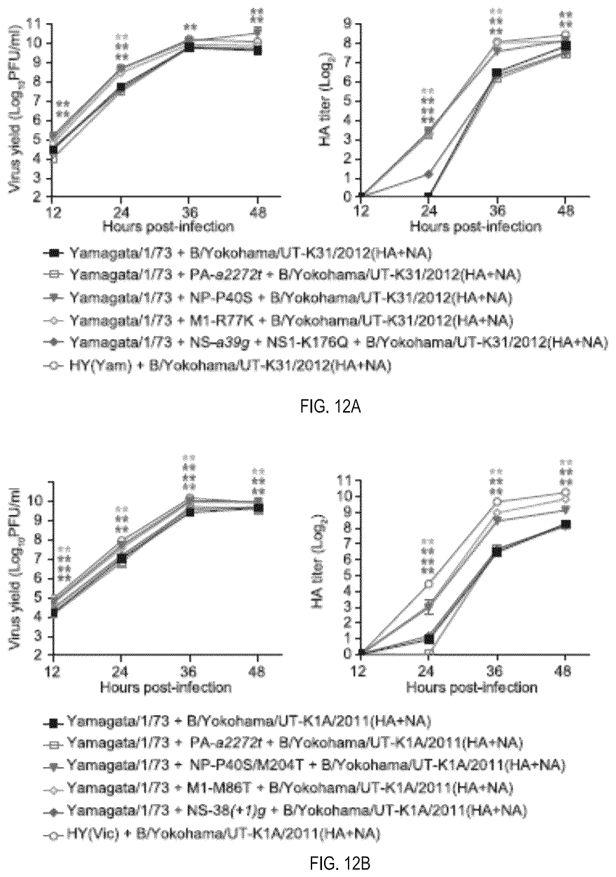

Contribution of Individual vRNAs to High-Yield Properties of HY(Yam) and HY(Vic). The HY(Yam) and HY(Vic) vaccine backbones possess mutations in several vRNA. To better understand the contributions of these mutations to the HY(Yam) and HY(Vic) phenotypes, reverse genetics was used to generate viruses possessing individual mutant vRNA segments of HY(Yam) or HY(Vic): for example, viruses were generated in which the HA and NA vRNAs of a Yamagata-lineage virus were combined with the six remaining vRNAs of wild-type B/Yamagata/1/73 (used to generate virus libraries), of wild-type B/Yamagata/1/73 (also encoding the NP-P40S mutation found in HY(Yam)), or of HY(Yam) (FIG. 12). The resulting viruses were tested for their replicative abilities and hemagglutination titers in MDCK cells (FIG. 12). When tested individually, the NP-P40S, M1-R77K, and NS-a39g+NS1-K176Q mutations significantly increased the viral and hemagglutination titers of Yamagata-linage viruses at one or more time points compared with the reference virus. For Victoria-lineage viruses, each of the mutations tested had a statistically significant growth-enhancing effect at one or more time points. Most of the viruses possessing individual mutations found in HY(Yam) or HY(Vic) did not replicate as efficiently as the high-yield vaccine candidates, demonstrating that combinations of several mutations are important for the high-yield properties of HY(Yam) and HY(Vic).

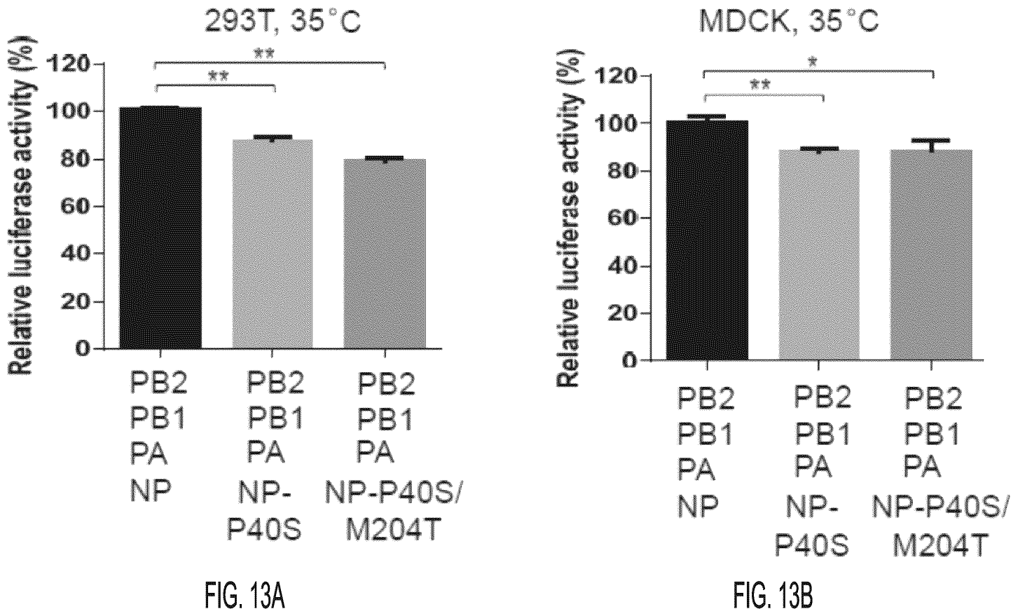

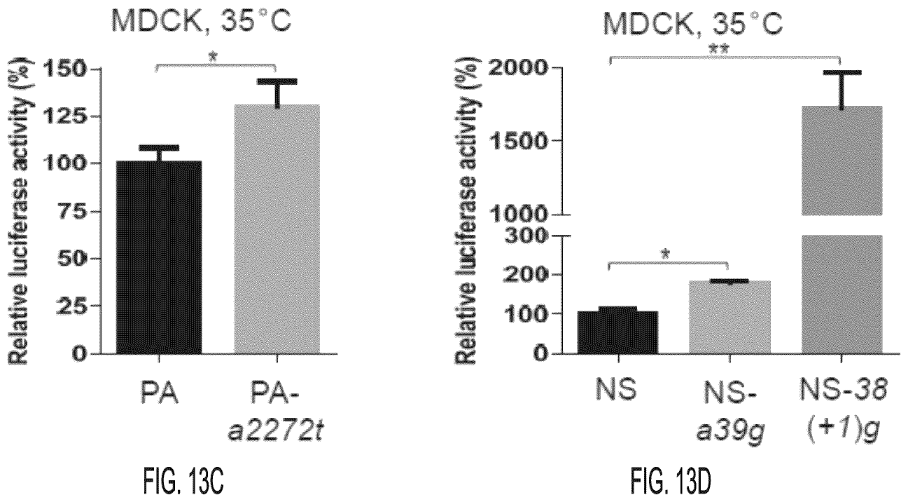

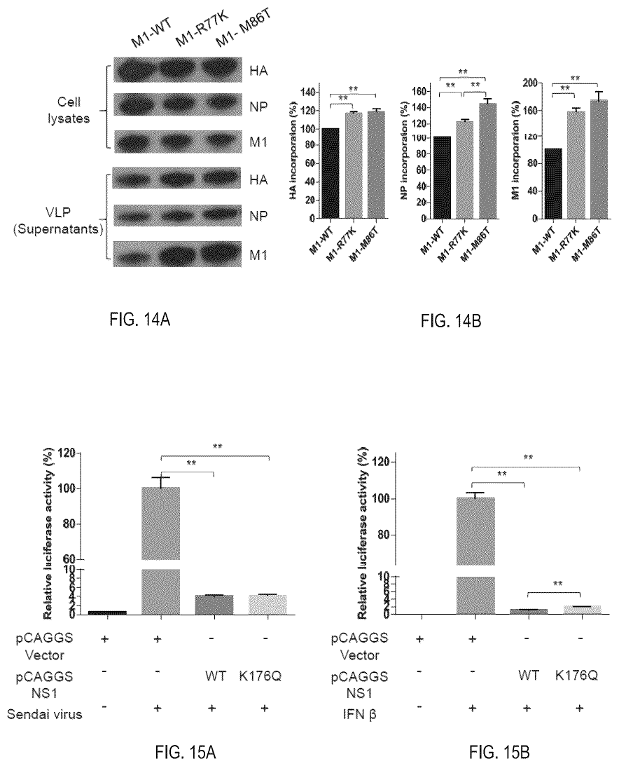

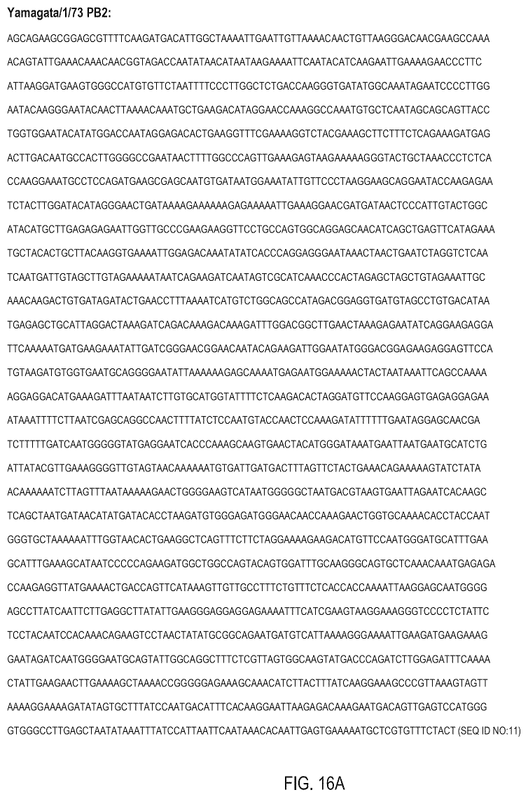

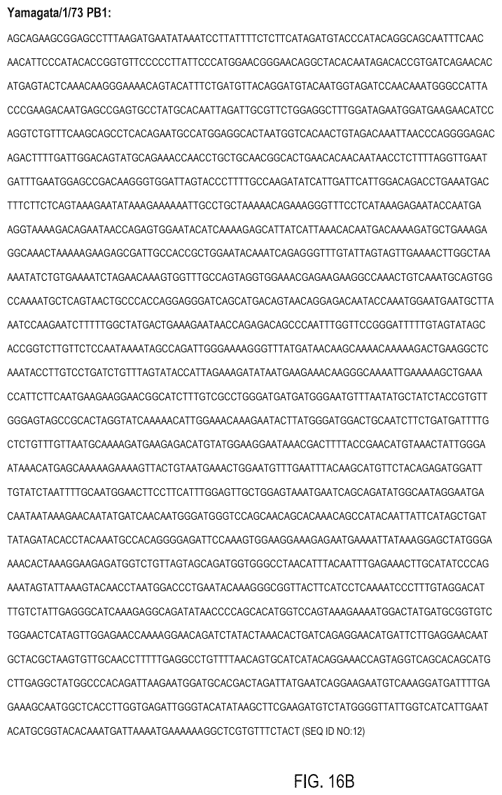

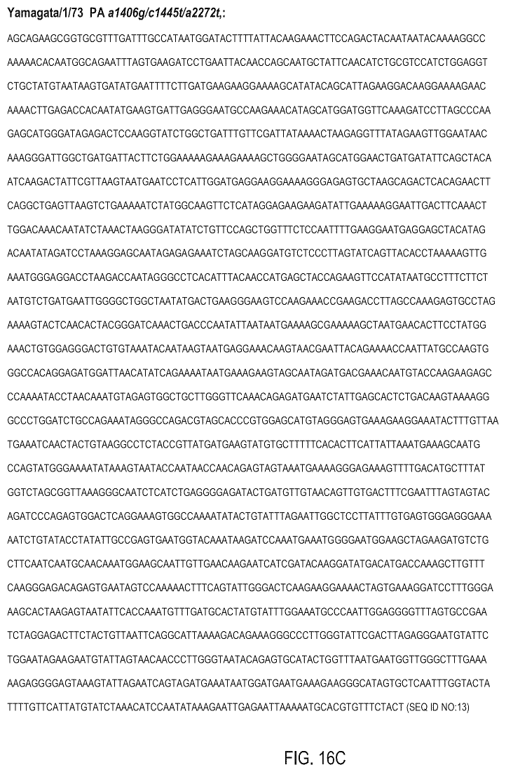

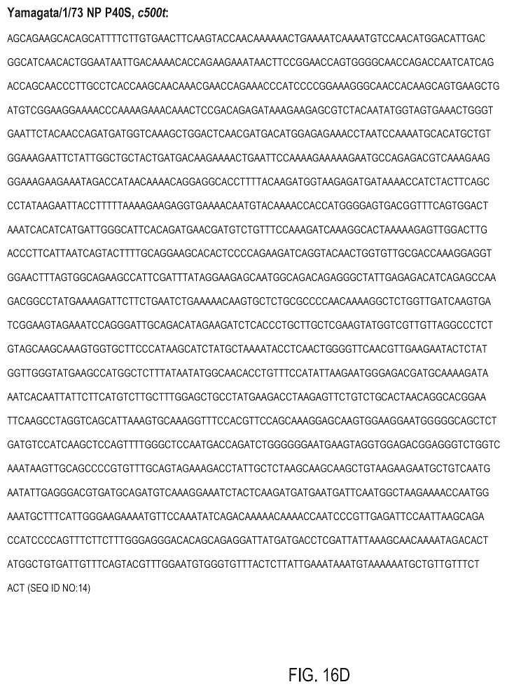

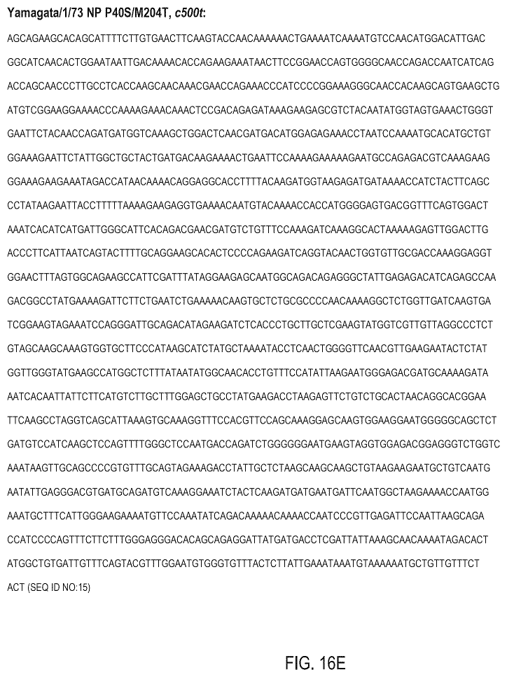

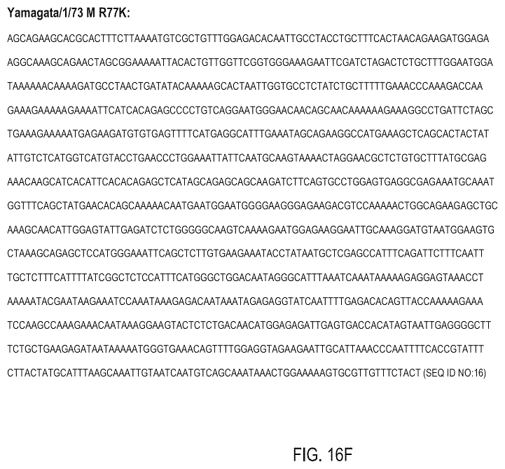

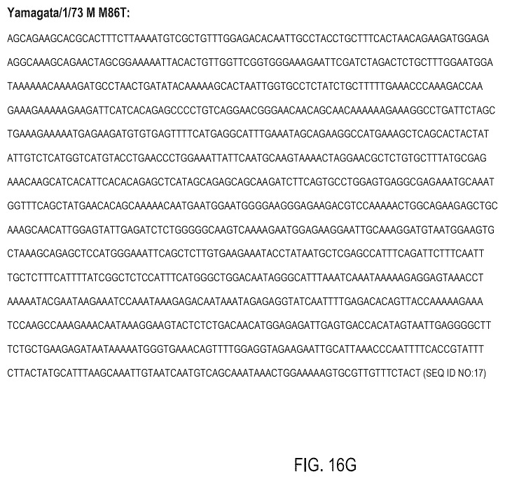

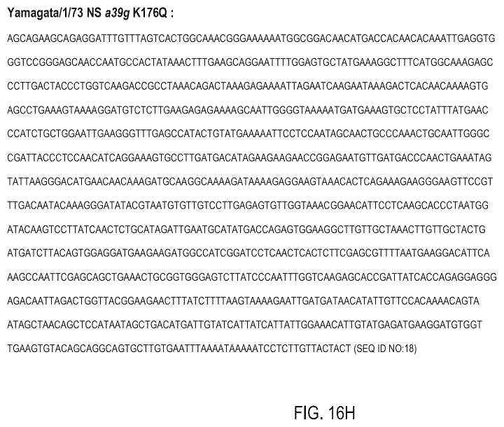

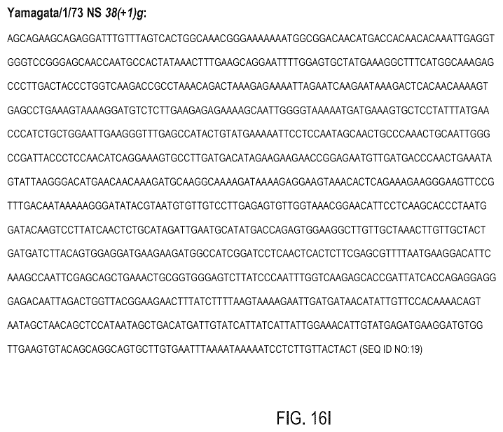

Effect of Individual Mutations in HY(Yam) and HY(Vic) on the Activity of the Viral Replication Complex. The NP-P40S mutation was selected individually and in combination with NP-M204T from the Yamagata- and Victoria-lineage libraries, respectively. Moreover, these mutations increased viral and hemagglutination titers when tested without the other growth-enhancing mutations (FIG. 12). To assess whether these mutations affect the activity of the viral replication complex, 293T and MDCK cells were transfected with plasmids expressing the three polymerase subunits of B/Yamagata/1/73 virus, wild-type or mutant B/Yamagata/1/73 NP, and an influenza B virus-like RNA expressing the luciferase reporter protein (the NP-M204T mutant was not tested separately because it was selected from the virus libraries only in combination with NP-P40S). Interestingly, the NP-P40S and -P40S/M204T mutations significantly reduced the replicative activity of the viral replication complex (FIG. 13A-B).

The PA-a2272t mutation emerged during passages of virus libraries in Vero cells (Tables 5 and 6) and significantly increased the viral and hemagglutination titers of a Victoria-lineage vaccine candidate (FIG. 6). This mutation was found to significantly increase the replication of a virus-like RNA in minireplicon assays (FIG. 105C). HY(Yam) and HY(Vic) possess NS-a39g and NS-38(+1)g mutations, respectively. These mutations were introduced into a virus-like RNA that expresses luciferase. Both mutations conferred significantly increased expression of the reporter protein from the virus-like RNA (FIG. 13D); this effect was substantially greater for the NS-38(+1)g mutation compared with the NS-a39g mutation.

Effect of Individual Mutations in HY(Yam) and HY(Vic) on the Composition of Virus-Like Particles. The M1-R77K and M1-M86T mutations in HY(Yam) and HY(Vic) affected viral and hemagglutination titers when tested individually (FIG. 12). M1 is the major structural component of virions and mutations in this protein could affect the composition of virions. 293T cells were transfected with plasmids for the expression of the B/Yamagata/1/73 HA, NA, NP, BM2, NS2, and wild-type or mutant M1 proteins; expression of this set of viral protein results in efficient virus-like particle (VLP) formation and release (Gomez-Puertas et al., 1999). Cell culture supernatant was collected and the VLP incorporation efficiency of viral proteins assessed (FIG. 14). Both mutations in M1 significantly increased the amount of viral HA, NP, and M1 proteins in the culture supernatant, which presumably resulted in the increased viral and hemagglutination titers conferred by the M1-R77K and -86T mutations.

Effect of a Mutation in the HY(Yam) NS1 Protein on IFN Antagonist Activity. The NS1 protein is the major influenza viral IFN antagonist (Yuan et al., 2001; Dauber et al., 2004). To assess the effect of the HY(Yam) NS1-K176Q mutation on NS1's ability to interfere with IFN-β synthesis, 293T cells were transfected with wild-type or mutant NS1 protein expression plasmids and with the reporter plasmid pGL-IFN-β, which encodes the firefly luciferase protein under the control of the IFN-β promoter (Bale et al., 2012). Cells were then infected with Sendai virus to stimulate IFN-β synthesis, resulting in increased reporter gene expression. Wild-type and mutant NS1 were comparable in their ability to down-regulate IFN-β synthesis (FIG. 12A). To determine the ability of wild-type and mutant NS1 to interfere with the expression of IFN-β-stimulated genes, 293T cells were transfected with wild-type or mutant NS1 protein expression plasmids and the reporter plasmid pISRE-Luc (Promega), which encodes the firefly luciferase protein under the control of an IFN-regulated promoter. Cells were stimulated with IFN-β 24 hours later, incubated again for 24 hours, and then assayed for luciferase expression. The NS1-K176Q protein was slightly less efficient than wild-type NS1 in suppressing gene synthesis from an IFN-regulated promoter (FIG. 12B), suggesting that other mechanisms account for its growth-enhancing effect.

### DISCUSSION

To date, no systematic efforts have been carried out to develop a high-yield influenza B vaccine backbone. Vodeiko et al. (Vodeiko et al., 2003) compared two influenza B viruses that differed in their replicative ability in embryonated chicken eggs. Reassortment experiments revealed several vRNAs that contributed to the phenotypic differences (Vodeiko et al., 2003); however, the specific amino acids that determined the growth properties were not identified. Kim et al. (Kim et al., 2015) found that coldadaptation of influenza B viruses enhanced their growth properties. Several amino acid changes in HA, NA, and NP (not the mutations reported here) were responsible for the increased virus titers (Kim et al., 2015). Le et al. (2015) tested reassortants between B/Lee/40 and Yamagata- and Victoria-lineage influenza B viruses isolated in 2002-2007; all 14 high-yield candidates possessed the NP vRNA of B/Lee/40 virus, suggesting that this vRNA confers efficient replicative ability. Ping et al. (2015) published a more comprehensive strategy to develop high-yield influenza A vaccine backbones, which was applied here to influenza B viruses: from virus libraries possessing random mutations in the internal genes, candidates were selected that improved influenza B virus replication in MDCK and Vero cells. Combinations of mutations resulted in Yamagata- and Victoria-lineage vaccine candidates (encoding the NP-P40S, M1-R77K, NS1-K176Q, NS-a39g, and PA-a2272t mutations for the Yamagata-lineage vaccine and the NP-P40S/M204T, M1-M86T, NS-(38+1)g, and PA-a2272t mutations for the Victoria-lineage vaccine) with high yield in MDCK and Vero cells, and also in embryonated chicken eggs. Further studies in (semi)industrial settings would be necessary to determine whether lineage-specific vaccine backbones provide an advantage over a single backbone used for viruses of both lineages.

The total viral protein yield and HA content obtained from HY(Yam) and HY(Vic) were substantially higher than those from wild-type viruses (FIG. 10). High virus and HA yields are important for cost-effective vaccine production. More importantly, increases in vaccine virus yield may be imperative in years with high-vaccine demand and in years with shortened Virus yield vaccine production times. However, some of the observed differences in virus titers or HA yield were small (although statistically significant), and we currently do not know the extent by which HY(Yam) and HY(Vic) would increase vaccine virus yield in industrial vaccine production.

Evaluation of influenza B virus sequences revealed that the amino acid changes in HY(Yam) and HY(Vic) are rare among natural influenza B viruses (the NP-P40S, NP-M204T, and M1-M86T mutations have each been found in one isolate; the M1-R77K mutation has been reported in 14 isolates; and the NS1-K176Q mutation has not been detected). The 3D structure of an influenza B virus NP protein has been resolved (Ng et al., 2012), but position 40 (at which P-to-S mutations were selected from the Yamagata- and Victoria-lineage libraries) is part of the N-terminal 71 amino acids for which no structural data could be obtained, suggesting that this region is highly flexible. Sequence analysis of influenza B virus NP proteins revealed that the proline at position 40 is highly conserved: only 1 in 3,234 sequences does not encode NP-40P (the only exception, B/Tennessee/01/2015, encodes NP-40S, as found in this study). Interestingly, the NP P40S mutation reduced the activity of the viral replication complex in minireplicon assays, suggesting that the yield-enhancing effect of this mutation is mediated by functions other than replication and transcription. For example, this region in NP could interact with viral or cellular proteins during the export of viral ribonucleoprotein complexes from the nucleus to the cytoplasm, or during virion assembly.

Several mutations were also identified in the noncoding regions of vRNA segments that increased virus yield; some of these mutations conferred increased levels of replication and transcription, as measured in minireplicon assays. These mutations may, for example, increase the stability of the vRNA or affect its interaction with the viral polymerase complex. Further studies are needed to decipher the exact mechanistic functions of these mutations.

Collectively, influenza B vaccine virus backbones were developed that could increase the titers of seasonal influenza B vaccines in the propagation systems currently used for human influenza vaccine virus production.

TABLE 1
Amino acid changes of selected Yamagata lineage high yield clones.

| Yamagata lineage | | | | | | | | | |
|---|---|---|---|---|---|---|---|---|---|
| HY clones # | HA | NA | PB2 | PB1 | PA | NP | M1 | BM2 | NS1 |
| #18, #19 | | | | | | | G34V, | H58R, | |
| | | | | | | | 197N | R80G | |
| #28 | K129E | | | | | | G34V, | H58R, | |
| | | | | | | | I97N | R80G | |
| #23 | N168D | | | | | | G34V, | H58R, | M117Y, |
| | | | | | | | I97N | R80G | S252T |
| #26 | | G434E | | | | | G34V, | H58R, | M117Y, |
| | | | | | | | I97N | R80G | S252T |
| #8 | | G434E | | | | | G34V, | H58R, | |
| | | | | | | | I97N | R80G | |
| #27 | | | | | | S57G | G34V, | H58R, | |
| | | | | | | | I97N | R80G | |
| #21 | | | | | | E52K | R77K | | |

TABLE 2
Amino acid changes of selected Victoria lineage high yield clones.

| Victoria lineage | | | | | | | | |
|---|---|---|---|---|---|---|---|---|
| HY clones # | HA | NA | PB2 | PB1 | PA | NP | M1 | NS1 |
| #2 | T196P | | | | | P40S, | M86T | |
| | | | | | | M204T | | |
| #4 | T196I | | | | | P40S, | M86T | |
| #27 | T196I | | | | | P40S | M86T | a39g, K176Q |
| #20 | T196A | | | | | P40S | D54G, M86T | Additional g |
| | | | | | | | | insert after |
| | | | | | | | | position 38* |
| #5 | T196P | | | | | P40S, | M86T | Additional g |
| | | | | | | P51Q | | insert after |
| | | | | | | | | position 38 |
| #25 | T34I, | | | | | A28T, | M86T | Additional g |
| | T196N | | | | | P40S, | | insert after |
| | | | | | | g1795a | | position 38 |
| #22 | T196P | N169T | | | | P40S, | M86T | Additional g |
| | | | | | | g1795a | | insert after |
| | | | | | | | | position 38 |
| #28 | | | | | | P40S, | M86T | Additional g |
| | | | | | | g1795a | | insert after |
| | | | | | | | | position 38 |

*Also referred to as 38(+1)g
Mutations in lowercase and italics in this and the following tables indicate nucleotide changes in the non-coding region

TABLE 3
Amino acid changes of Yarnagata lineage HY vaccine virus
candidates generated by using reverse genetics.

| Yamagata | | | | | | | | |
|---|---|---|---|---|---|---|---|---|
| lineage | | | | | | | | |
| canidates | HA &NA | PB2 | PB1 | PA | NP | M1 | BM2 | NS1 |
| RG(Yam)# 1 | B/Yokohama/UT- | | | | E52K | R77K | | |
| | k31/2012 | | | | | | | |
| RG(Yam)# 2 | (Yamagata | | | | E52K | R77K | | M117Y, |
| | lineage) | | | | | | | S252T |
| RG(Yam)# 3 | | | | | E52K | G34V, | H58R, | |
| | | | | | | I97N | R82G | |
| RG(Yam)# 4 | | | | | S57G | R77K | | M117Y, |
| | | | | | | | | S252T |
| RG(Yam)# 5 | | | | | P40S, | M86T | | Additional g |
| | | | | | g1795a | | | insert after |
| | | | | | | | | position 38 |
| RG(Yam)# 6 | | | | | P40S | D54G, | | Additional g |
| | | | | | | M86T | | insert after |
| | | | | | | | | position 38 |
| RG(Yam)# 7 | | | | | E52K | D54G, | | M117Y, |
| | | | | | | M86T | | S252T |
| RG(Yam)# 8 | | | | | P40S | R77K | | a39g, |
| | | | | | | | | K176Q |

TABLE 4
Amino acid changes of Victoria lineage HY vaccine virus
candidates generated by using reverse genetics.

| Victoria | | | | | | | | |
|---|---|---|---|---|---|---|---|---|
| lineage | | | | | | | | |
| candidates | HA &NA | PB2 | PB1 | PA | NP | M1 | BM2 | NS1 |
| RG(Vic)#1 | B/Yokohama/UT- | | | | P40S | D54G, | | Addonal g insert |
| | K1A/2011 | | | | | M86T | | after position 38 |
| RG(Vic)#2 | (Victoria lineage) | | | | P40S | M86T | | Addonal g insert |
| | | | | | M204T | | | after position 38 |
| RG(Vic)#3 | | | | | P40S, | M86T | | Addonal g insert |
| | | | | | g1795a | | | after position 38 |
| RG(Vic)#4 | | | | | P40S, | M86T | | Addonal g insert |
| | | | | | P51Q | | | after position 38 |
| RG(Vic)#5 | | | | | P40S | M86T | | a39g, K176Q |
| RG(Vic)#6 | | | | | E52K | R77K | | |
| RG(Vic)#7 | | | | | E52K | D54G, | | M117Y, S252T |
| | | | | | | M86T | | |
| RG(Vic)#8 | | | | | P40S | D54G, | | Addonal ga |
| | | | | | | M86T | | insert after |
| | | | | | | | | position 38 |

Virus libraries were passaged for a total of 12 times in MDCK cells, e.g., 2 passages after which the libraries may be mixed and then 10 more passages were carried out (FIG. 2). After passages in MDCK cells, plaque assays were performed and over 1,400 individual plaques were picked. High-yield candidates are shown in Tables 1-4.

Tables 5 and 6 show changes that resulted in enhanced growth in Vero cells using a similar protocol.

TABLE 5
Amino acid changes of Vero cell adapted HY Yamagata lineage viruses.

| Yamagata | | | | | | | | | | |
|---|---|---|---|---|---|---|---|---|---|---|
| lineage | | | | | | | | | | |
| clone | HA1 | HA2** | NA | PB2 | PB1 | PA | NP | M1 | BM2 | NS1 |
| 1 | N194D | D112E | T436M | | | | | G34N, | H58R | |
| | | | | | | | | I97N | | |
| 2 | T196P | D112E | E105K | N16S | | D494N | | G34N, | H27R, | |
| | | | | | | | | I97N | H58R | |
| 3 | | D112E | T76M, P139S, | N16S | | D494N | | G34N, | H27R, | |
| | | | D457N | | | | | I97N | H58R | |
| 4 | | D112E | R102K, T436M | | | a2272t | | G34N, | H58R | |
| | | | | | | | | I97N | | |
| 5 | | D112E | T436M | | | a2272t | P343T | G34N, | H58R | |
| | | | | | | | | I97N | | |
| 6 | | D112E | | | | g2213a, | P343T | G34N, | G28R, | |
| | | | | | | a2272t | | I97N | H58R | |

**numbering begins at cleavage site

TABLE 6
Amino acid changes of Vero cell adapted HY Victoria lineage viruses.

| Victoria | | | | | | | | | | |
|---|---|---|---|---|---|---|---|---|---|---|
| lineage clone | HA1 | HA2** | NA | PB2 | PB1 | PA | NP | M1 | BM2 | NS1 |
| 1 | T196A, | S56G, | D457N | | | Y387H, | | | R80G | Y42N |
| | | K61N | | | | V434A, | | | | |
| | | | | | | T524A, | | | | |
| | | | | | | a2272t | | | | |
| 2 | T196A, | K39G, | D457N | | | Y387H, | | | R80G | Y42N |
| | | S56G | | | | V434A, | | | | |
| | | | | | | a2272t | | | | |
| 3 | T196A | S56G, | D457N | | | Y387H, | | | R80G | Y42N |
| | | K61N | | | | V434A, | | | | |
| | | | | | | a2272t | | | | |
| 4 | R98K, | S56G | D457N | | | Y387H, | | | R80G | Y42N |
| | T196A | | | | | V434A, | | | | |
| | | | | | | a2272t | | | | |
| 5 | T196A | K39G, | D457N | | | Y387H, | | | R80G | Y42N |
| | | S56G | | | | V434A, | | | | |
| | | | | | | a2272t | | | | |
| 6 | T196A | S56G | D457N | | | Y387H, | | | R80G | Y42N |
| | | | | | | V434A, | | | | |
| | | | | | | a2272t | | | | |

**numbering begins at cleavage site

TABLE 7
Amino acid changes detected after serial virus passages
in embryonated chicken eggs

| | Egg | |
|---|---|---|
| Virus | passage | Amino acid changes in HA |
| B/Massachusetts/2/2012 | P1 | None |
| | P2 | None |
| | P3 | None |
| | P4 | None |
| | P5 | None |
| | P6 | None |
| | P7 | None |
| | P8 | None |
| | P9 | None |
| | P10 | None |
| HY(Yam) + | P1 | None |
| B/Massachusetts/2/2012 | P2 | None |
| (HA + NA) | P3 | None |
| | P4 | None |
| | P5 | None |
| | P6 | None |
| | P7 | None |
| | P8 | None |
| | P9 | None |
| | P10 | None |
| B/Brisbane/60/2008 | P1 | None |
| | P2 | None |
| | P3 | None |
| | P4 | None |
| | P5 | None |
| | P6 | None |
| | P7 | None |
| | P8 | None |
| | P9 | None |
| | P10 | None |
| HY(Vic) + | P1 | None |
| B/Brisbane/60/2008 | P2 | None |
| (HA + NA) | P3 | None |
| | P4 | None |
| | P5 | None |
| | P6 | None |
| | P7 | None |
| | P8 | None |
| | P9 | None |
| | P10 | None |
| B/Yokohama/UT-K31/2012 | P1 | None |
| | P2 | N194K (loss of glycosylation |
| | | site) |
| | P3 | N194K (loss of glycosylation |
| | | site) |
| | P4 | N194K (loss of glycosylation |
| | | site) |
| | P5 | N194K (loss of glycosylation |
| | | site) |
| HY(Yam) + | P1 | None |
| B/Yokohama/UT- | P2 | N194N/S (N194S results in loss |
| K31/2012(HA + NA) | | of glycosylation site) |
| | P3 | N194S (loss of glycosylation site) |
| | P4 | N194S (loss of glycosylation site) |
| | P5 | N194S (loss of glycosylation site) |
| B/Yokohama/UT-K1A/ | P1 | None |
| 2011 | P2 | T196I (loss of glycosylation site) |
| | P3 | T196I (loss of glycosylation site) |
| | P4 | T196I (loss of glycosylation site) |
| | P5 | T196I (loss of glycosylation site) |
| Hy(Vic) + | P1 | None |
| B/Yokohama/UT- | P2 | T196T/I (T196I results in loss of |
| K1A/2011(HA + NA) | | glycosylation site) |
| | P3 | T196T/I (T196I results in loss of |
| | | glycosylation site) |
| | P4 | T196I (loss of glycosylation site) |
| | P5 | T196I (loss of glycosylation site) |
| Hy(Vic) + B/ | P1 | None (MDCK passages already |
| Yokohama/UT-K1A/ | | resulted in loss of glycosylation |
| 2011(HA + NA) | | site at positions 194-196) |
| (10 sequential | P2 | None (MDCK passages already |
| passages in | | resulted in loss of glycosylation |
| MDCK cells) | | site at positions 194-196) |
| | P3 | None (MDCK passages already |
| | | resulted in loss of glycosylation |
| | | site at positions 194-196) |
| | P4 | None (MDCK passages already |
| | | resulted in loss of glycosylation |
| | | site at positions 194-196) |
| | P5 | None (MDCK passages already |
| | | resulted in loss of glycosylation |
| | | site at positions 194-196) |
| B/Yokohama/P-2922/2005 | P1 | None |
| | P2 | N194D (loss of glycosylation |
| | | site) |
| | P3 | N194D (loss of glycosylation |
| | | site) |
| | P4 | N194D (loss of glycosylation |
| | | site) |
| | P5 | N194D (loss of glycosylation |
| | | site) |
| HY(Yam) + B/ | P1 | None |
| Yokohama/P-2922/2005 | P2 | N194D (loss of glycosylation |
| | | site) |
| | P3 | N194D (loss of glycosylation |
| | | site) |
| | P4 | N194D (loss of glycosylation |
| | | site) |
| | P5 | N194D (loss of glycosylation |
| | | site) |
| B/Tokyo/UTE2/2008 | P1 | T196I (loss of glycosylation site) |
| | P2 | T196I (loss of glycosylation site) |
| | P3 | T196I (loss of glycosylation site) |
| | P4 | T196I (loss of glycosylation site) |
| | P5 | T196I (loss of glycosylation site) |
| HY(Vic) + | P1 | None |
| B/Tokyo/UTE2/2008 | P2 | N194S (loss of glycosylation site) |
| (HA + NA) | P3 | N194S (loss of glycosylation site) |
| | P4 | N194S (loss of glycosylation site) |
| | P5 | N194S (loss of glycosylation site) |

### REFERENCES

Ambrose and Levin, Hum. Vaccin. Immunother. 8:81 (2012).
Avery's Drug Treatment: Principles and Practice of Clinical Pharmacology and Therapeutics, 3rd edition, ADIS Press, Ltd., Williams and Wilkins, Baltimore, MD (1987).
Aymard-Henry et al., Virology: A Practical Approach, Oxford IRL Press, Oxford, 119-150 (1985).
Bachmeyer, Intervirology, 5:260 (1975).
Baker et al., J. Virol., 88:10778 (2014).
Bale et al., PLoS Pathog., 8:e1002916 (2012).
Belshe et al., Influenza Other Respi. Viruses, 4:141 (2010).
Belshe, Vaccine, 28:D45 (2010).
Berkow et al., eds., The Merck Manual, 16th edition, Merck & Co., Rahway, NJ (1992).
Dauber et al., J. Virol., 78:1865 (2004).
Flandorfer et al., J. Virol., 77:9116 (2003).
Fujii et al., Proc. Natl. Acad. Sci. U.S.A., 100:2002 (2003).
Glezen et al., Am. J. Public Health. 103:e43 (2013).
Gómez-Puertas et al., J. Gen. Virol., 80:1635 (1999).
Hatta et al., Science, 293:1840 (2001).
Horimoto et al., J. Virol., 68:3120 (1994).
Horimoto et al., Microbes Infect., 6:579 (2004).
Horimoto et al., Vaccine, 24:3669 (2006).
Keitel et al., in Textbook of Influenza, eds. Nickolson, K. G., Webster, R. G., and Hay, A. (Blackwell, Oxford), pp. 373-390 (1998).
Kim et al., Vaccine, 33:5786 (2015).
Knipe et al., (Lippincott Williams & Wilkins, Philadelphia, PA), 6th Ed, vol 1, pp 1186-1243.
Laver & Webster, Virology, 69:511 (1976).
Le et al., Vaccine, 33:879 (2015).
Maassab, J. Immunol., 102:728 (1969).
Neumann et al., Adv. Virus Res., 53:265 (1999).
Neumann et al., J. Gen. Virol., 83:2635 (2002).
Neumann et al., J. Virol., 71:9690 (1997).
Neumann et al., Proc. Natl. Acad. Sci. USA, 96:9345 (1999).
Neumann et al., Virology, 287:243 (2001).
Ng, et al., J. Virol., 86:6758 (2012).
Osol (ed.), Remington's Pharmaceutical Sciences. Mack Publishing Co., Easton, PA 1324-1341 (1980).
Ping et al., Nat. Commun., 6:8148 (2015).
Ramanunninair, et al. PLoS One, 8:e65955 (2013).
Rota, et al., Virology, 175:59 (1990).
Sugawara et al., Biologicals, 30:303 (2002).
Tafalla et al., Hum. Vaccin. Immunother., 12:993002 (2016).
van de Sandt et al., Future Microbiol., 10:1447 (2015).
Vodeiko et al., Vaccine. 21:3867 (2003).
Webby & Webster et al., Science, 302:1519 (2003).
Wood & Robertson, Nat. Rev. Microbiol., 2:842 (2004).
World Health Organization TSR No. 673 (1982).
World Health Organization. Confirmed human cases of avian influenza A (H5N1). http://www.who.int/csr/disease/avian_influenza/country/en/index.html
Wright et al., Fields Virology, eds (2013). Orthomyxoviruses.
Yuan and Krug, EMBO. J., 20:362 (2001).

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1-20. (canceled)

21. A composition comprising a vector for influenza B virus NP vRNA, cRNA or mRNA expression, wherein the influenza B virus NP has at least 85% amino acid sequence identity to a polypeptide encoded by SEQ ID NO:4, 14 or 15 and has S at position 40, or S or T at position 40 and T, V, L, I or A at position 204.

22. The composition of claim 21 wherein the vector is for influenza B virus NP vRNA, or cRNA expression.

23. The composition of claim 21 wherein the vector for vRNA or cRNA expression comprises a promoter operably linked to an influenza virus NP DNA linked to a transcription termination sequence

24. The composition of claim 21 wherein the influenza B virus NP has at least 90% amino acid sequence identity to a polypeptide encoded by SEQ ID NO:4, 14 or 15.

25. The composition of claim 23 further comprising a vector for vRNA or cRNA production comprising a promoter operably linked to an influenza virus PA DNA linked to a transcription termination sequence, a vector for vRNA or cRNA production comprising a promoter operably linked to an influenza virus PB1 DNA linked to a transcription termination sequence, a vector for vRNA or cRNA production comprising a promoter operably linked to an influenza virus PB2 DNA linked to a transcription termination sequence, a vector for vRNA or cRNA production comprising a promoter operably linked to an influenza virus HA DNA linked to a transcription termination sequence, a vector for vRNA or cRNA production comprising a promoter operably linked to an influenza virus NA DNA linked to a transcription termination sequence, a vector for vRNA or cRNA production comprising a promoter operably linked to an influenza virus M DNA linked to a transcription termination sequence, and a vector for vRNA or cRNA production comprising a promoter operably linked to an influenza virus NS DNA linked to a transcription termination sequence, wherein the PB1, PB2, PA, NP, NS, and M DNAs in the vectors for vRNA or cRNA production are from one or more influenza vaccine virus isolates.

26. The composition of claim 25 wherein the NA DNA in the vector for vRNA or cRNA production of NA has sequences for a heterologous or chimeric NA, and wherein the HA DNA in the vector for vRNA or cRNA production of HA has sequences for a heterologous or chimeric HA, wherein the M viral segment encodes a M1 polypeptide with a lysine or histidine at position 77 or a M1 polypeptide with a threonine, glycine, valine, leucine, isoleucine or alanine at position 86, wherein the NS viral segment encodes a NS1 polypeptide with a glutamine or asparagine at position 176, and optionally wherein the NS vRNA or cRNA encodes a NS1 polypeptide having a residue other than Y at position 42, other than M at position 117, and/or other than S at position 252, and/or a nucleotide other than an a at position 39 or a nucleotide insertion after position 38, or any combination thereof; or optionally the M vRNA or cRNA encodes a M1 polypeptide having a residue other than G at position 34, other than D at position 54, other than 1 at position 97, or any combination thereof; or optionally encoded by the M vRNA or cRNA encodes a BM2 polypeptide having a residue other than H at position 58, other than R at position 80, other than H at position 27, other than G at position 26, or any combination thereof; or optionally the NP vRNA or cRNA has a nucleotide other than g at position 1795 or other than c at position 500, or any combination thereof; or optionally the PA vRNA or cRNA encodes a PA polypeptide having a residue other than Y at position 387, other than V at position 434, other than D at position 494, and/or other than T at position 524, and/or the PA vRNA or cRNA has a nucleotide other than a at nucleotide 2272, other than a at position 1406, other than c at position 1445, and/or other than g at nucleotide 2213, or any combination thereof; or optionally the PB2 vRNA or cRNA encodes a PB2 polypeptide has a residue other than N at position 16, wherein the position in the NS polypeptide is relative to a NS polypeptide encoded by SEQ ID NO: 6, wherein the position in the M1 polypeptide is relative to a M1 polypeptide encoded by SEQ ID NO:5, wherein the position in the BM2 polypeptide is relative to a BM2 polypeptide encoded by SEQ ID NO:5, wherein the position in the PA polypeptide is relative to a PA polypeptide encoded by SEQ ID NO:3, or wherein the position in the PB2 polypeptide is relative to a PB2 polypeptide encoded by SEQ ID NO: 1; or any combination thereof.

27. The composition of claim 25 wherein optionally the NP vRNA has u at nucleotide 500, and the M1 polypeptide has K at position 77, the NS1 polypeptide has Q at position 176 and the NS vRNA has g at nucleotide 39, or the PA vRNA has g at nucleotide 1406, u at nucleotide 1445, and u at nucleotide 2272, or any combination thereof.

28. The composition of claim 25 wherein optionally the NP vRNA has u at nucleotide 500, and the M1 polypeptide has T at position 86, the NS vRNA has an insertion of g after nucleotide 38, or the PA vRNA has g at nucleotide 1406, u at nucleotide 1445, and u at nucleotide 2272, or any combination thereof.

29. A method of introducing a mutation into a vector for influenza B virus NP vRNA, cRNA or mRNA expression, comprising introducing into the NP coding region of the vector a S at position 40, or S or T at position 40 and T, V, L, I or A at position 204 in NP.

30. A plurality of vectors comprising a vector for vRNA or cRNA production comprising a promoter operably linked to an influenza virus PA DNA linked to a transcription termination sequence, a vector for vRNA or cRNA production comprising a promoter operably linked to an influenza virus PB1 DNA linked to a transcription termination sequence, a vector for vRNA or cRNA production comprising a promoter operably linked to an influenza virus PB2 DNA linked to a transcription termination sequence, a vector for vRNA or cRNA production comprising a promoter operably linked to an influenza virus HA DNA linked to a transcription termination sequence, a vector for vRNA or cRNA production comprising a promoter operably linked to an influenza virus NP DNA linked to a transcription termination sequence, a vector for vRNA or cRNA production comprising a promoter operably linked to an influenza virus NA DNA linked to a transcription termination sequence, a vector for vRNA or cRNA production comprising a promoter operably linked to an influenza virus M DNA linked to a transcription termination sequence, and a vector for vRNA or cRNA production comprising a promoter operably linked to an influenza virus NS DNA linked to a transcription termination sequence, wherein the PB1, PB2, PA, NP, NS, and M DNAs in the vectors for vRNA or cRNA production are from one or more influenza vaccine virus isolates, wherein the NA DNA in the vector for vRNA or cRNA production of NA has sequences for a heterologous or chimeric NA, and wherein the HA DNA in the vector for vRNA or cRNA production of HA has sequences for a heterologous or chimeric HA, wherein the NP viral segment encodes a NP polypeptide having serine at position 40, or a serine or threonine at position 40 and a threonine, valine, leucine, isoleucine or alanine at position 204, wherein the M viral segment encodes a M1 polypeptide with a lysine or histidine at position 77 or a M1 polypeptide with a threonine, glycine, valine, leucine, isoleucine or alanine at position 86, wherein the NS viral segment encodes a NS1 polypeptide with a glutamine or asparagine at position 176, and optionally wherein the NS vRNA or cRNA encodes a NS1 polypeptide having a residue other than Y at position 42, other than M at position 117, and/or other than S at position 252, and/or a nucleotide other than an a at position 39 or a nucleotide insertion after position 38, or any combination thereof; or optionally the M vRNA or cRNA encodes a M1 polypeptide having a residue other than G at position 34, other than D at position 54, other than I at position 97, or any combination thereof; or optionally encoded by the M vRNA or cRNA encodes a BM2 polypeptide having a residue other than H at position 58, other than R at position 80, other than H at position 27, other than G at position 26, or any combination thereof; or optionally the NP vRNA or cRNA has a nucleotide other than g at position 1795 or other than c at position 500, or any combination thereof; or optionally the PA vRNA or cRNA encodes a PA polypeptide having a residue other than Y at position 387, other than V at position 434, other than D at position 494, and/or other than T at position 524, and/or the PA vRNA or cRNA has a nucleotide other than a at nucleotide 2272, other than a at position 1406, other than c at position 1445, and/or other than g at nucleotide 2213, or any combination thereof; or optionally the P132 vRNA or cRNA encodes a P132 polypeptide has a residue other than N at position 16, wherein the position in the NS polypeptide is relative to a NS polypeptide encoded by SEQ ID NO: 6, wherein the position in the M1 polypeptide is relative to a M1 polypeptide encoded by SEQ ID NO:5, wherein the position in the BM2 polypeptide is relative to a BM2 polypeptide encoded by SEQ ID NO:5, wherein the position in the PA polypeptide is relative to a PA polypeptide encoded by SEQ ID NO:3, or wherein the position in the PB2 polypeptide is relative to a PB2 polypeptide encoded by SEQ ID NO:1.

31. The plurality of vectors of claim 30 wherein the NP polypeptide has S at position 40 and optionally further has at least one of: T at position 28, Q at position 51, K at position 52, G at position 57, T at position 204, Tat position 343, a at position 1795 or the NP viral segment has t at position 500.

32. The plurality of vectors of claim 30 wherein the M1 polypeptide has at least one of: V or N at position 34, G at position 54, K at position 77, T at position 86, or N at position 97.

33. The plurality of vectors of claim 30 wherein the BM2 polypeptide has at least one of: R at position 58, G at position 80, R at position 27 or R at position 26.

34. The plurality of vectors of claim 30 wherein the NS1 polypeptide has at least one of: N at position 42, Y at position 117, Q at position 176, T at position 252, or the NS segment has a nucleotide insertion of g after nucleotide position 38 or g at position 39.

35. The plurality of vectors of claim 30 wherein the PA viral segment has at least one of: H at position 387, A at position 434, N at position 494, A at position 524, g at position 1406, t at position 2272, t at position 1445, or any combination thereof.

36. The plurality of vectors of claim 30 wherein the NP polypeptide has S at position 40 and optionally further comprises one or more of: the NP vRNA has u at nucleotide 500, the M1 polypeptide has K at position 77, the NS1 polypeptide has Q at position 176 and the NS vRNA has g at nucleotide 39, or the PA vRNA has g at nucleotide 1406, t at nucleotide 1445, and t at nucleotide 2272.

37. The plurality of vectors of claim 30 wherein the NP polypeptide has S at position 40 and T at position 204 and optionally further comprises one or more of the NP vRNA has u at nucleotide 500, the M1 polypeptide has T at position 86, the NS vRNA has an insertion of g after nucleotide 38, or the PA vRNA has g at nucleotide 1406, t at nucleotide 1445, and t at nucleotide 2272.

38. The plurality of vectors of claim 30 wherein the PA, PB1, PB2, NP, NS, and M viral segments comprise sequences encoding at least one of the following: a PB1 having the amino acid sequence encoded by SEQ ID NO:2 or PB1 with at least 80% amino acid sequence identity to the PB1 encoded by SEQ ID NO:2; a PB2 having the amino acid sequence encoded by SEQ ID NO:3 or PB2 with at least 80% amino acid sequence identity to the PB2 encoded by SEQ ID NO:3; a PA having the amino acid sequence encoded by SEQ ID NO:1 or PA with at least 80% amino acid sequence identity to the PA encoded by SEQ ID NO:1; a NP having the amino acid sequence encoded by SEQ ID NO:4 or NP with at least 80% amino acid sequence identity to the NP encoded by SEQ ID NO:4; a M having the amino acid sequence encoded by SEQ ID NO:5 or M with at least 80% amino acid sequence identity to the M encoded by SEQ ID NO:5; or a NS having the amino acid sequence encoded by SEQ ID NO: 6 or NS with at least 95% amino acid sequence identity to the NS encoded by SEQ ID NO:6.

39. The plurality of vectors of claim 30 wherein the M viral segment encodes a M1 polypeptide with a lysine or histidine at position 77 or a M1 polypeptide with a threonine, glycine, valine, leucine, isoleucine or alanine at position 86, wherein the NS viral segment encodes a NS1 polypeptide with a glutamine or asparagine at position 176, and optionally the NS viral segment encodes a NS1 polypeptide having a residue other than Y at position 42, other than M at position 117, and/or other than S at position 252, and/or the NS viral segment has a nucleotide other than a at nucleotide position 39 or a nucleotide insertion after position 38, or any combination thereof
